(19) United States
(12) Patent Application Publication
(10) Pub. No.: US 2020/0095236 A1
(43) Pub. Date: Mar. 26, 2020

(54) ANTIBACTERIAL COMPOUNDS

(71) Applicant: FORGE THERAPEUTICS, INC., San Diego, CA (US)

(72) Inventors: Min TENG, San Diego, CA (US); Baskar NAMMALWAR, San Diego, CA (US); Konstantin TAGANOV, San Diego, CA (US); Xiaoming LI, San Diego, CA (US); David T. PUERTA, San Diego, CA (US); Serge CONVERS-REIGNIER, Abingdon, Oxfordshire (GB); Holly ATTON, Abingdon, Oxfordshire (GB); Ian YULE, Abingdon, Oxfordshire (GB); Heather TYE, Abingdon, Oxfordshire (GB); Adele FAULKNER, Abingdon, Oxfordshire (GB); Alastair PARKES, Abingdon, Oxfordshire (GB)

(21) Appl. No.: 16/612,316

(22) PCT Filed: May 9, 2018
(86) PCT No.: PCT/US18/31898

§ 371 Date: Nov. 8, 2019

Related U.S. Application Data
(60) Provisional application No. 62/504,481, filed on May 10, 2017.

(51) Int. Cl. C07D 413/10; C07D 239/52

(52) U.S. Cl. CPC C07D 413/10; C07D 239/52

(57) ABSTRACT

Provided herein are heterocyclic derivative compounds and pharmaceutical compositions comprising said compounds that are useful for inhibiting the growth of gram-negative bacteria. Furthermore, the subject compounds and compositions are useful for the treatment of bacterial infection, such as urinary tract infection and the like.

### CROSS-REFERENCES TO RELATED APPLICATIONS

This application claims benefit of U.S. Patent Application No. 62/504,481, filed on May 10, 2017, which is hereby incorporated by reference in its entirety.

### BACKGROUND

A need exists in the medicinal arts for the effective treatment of illness caused by bacterial infection.

### BRIEF SUMMARY OF THE INVENTION

Provided herein are heterocyclic derivative compounds and pharmaceutical compositions comprising said compounds that are useful for inhibiting the growth of gram-negative bacteria. Furthermore, the subject compounds and compositions are useful for the treatment of bacterial infection, such as urinary tract infection and the like.

One embodiment provides a compound, or a pharmaceutically acceptable salt thereof, having the structure of Formula (I):

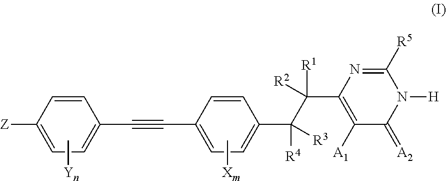

wherein,
n is 0-4;
m is 0-4;
A₁ is OH or SH;
A₂ is O or S;
R¹, R², R³ and R⁴ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹);
or R¹ and R² are taken together to form an optionally substituted alkenyl, ═NR¹¹, oxo, or thioxo;
or R¹ and R² are joined to form an optionally substituted carbocyclyl or optionally substituted heterocyclyl;
or R³ and R⁴ are taken together to form an optionally substituted alkenyl; ═NR¹¹, oxo, or thioxo;
or R³ and R⁴ are joined to form an optionally substituted carbocyclyl or optionally substituted heterocyclyl;
R⁵ is H, halogen, optionally substituted alkyl, hydroxyl, alkoxyl, cyano, amino, or nitro;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2;
and wherein if R¹ is unsubstituted (C₁-C₄ alkylene)-OH, then (i) at least one of R², R³, or R⁴ is not H; or (ii) n is 1-4 and m is 0-4; or (iii) n is 0-4 and m is 1-4;
and wherein if R² is unsubstituted (C₁-C₄ alkylene)-OH, then (i) at least one of R¹, R³, or R⁴ is not H; or (ii) n is 1-4 and m is 0-4; or (iii) n is 0-4 and m is 1-4.

One embodiment provides a compound, or a pharmaceutically acceptable salt thereof, having the structure of Formula (Ia):

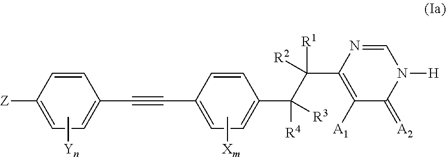

wherein,
n is 0, 1, or 2;
m is 0, 1, or 2;
A₁ is OH or SH;
A₂ is O or S;
R¹ and R³ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹);
R² is H or optionally substituted C₁-C₃ alkyl, provided that if R¹ is H, then R² is H or unsubstituted C₁-C₃ alkyl; or R¹ and R² are taken together to form an optionally substituted alkenyl; or R¹ and R² are joined to form an optionally substituted carbocyclyl or optionally substituted heterocyclyl;
R⁴ is H or optionally substituted C₁-C₃ alkyl; or R³ and R⁴ are taken together to form an optionally substituted alkenyl; or R³ and R⁴ are joined to form an optionally substituted carbocyclyl or optionally substituted heterocyclyl;
X is halogen, hydroxy, or optionally substituted C₁-C₃ alkyl;
Y is halogen, hydroxy, or optionally substituted C₁-C₃ alkyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2;
and wherein if R¹ is unsubstituted (C₁-C₄ alkylene)-OH, then (i) at least one of R², R³, or R⁴ is not H; or (ii) n is 1 or 2 and m is 0, 1, or 2; or (iii) n is 0, 1, or 2 and m is 1 or 2;
and wherein if R² is unsubstituted (C₁-C₄ alkylene)-OH, then (i) at least one of R¹, R³, or R⁴ is not H; or (ii) n is 1 or 2 and m is 0, 1, or 2; or (iii) n is 0, 1, or 2 and m is 1 or 2.

One embodiment provides a compound, or a pharmaceutically acceptable salt thereof, having the structure of Formula (IIa):

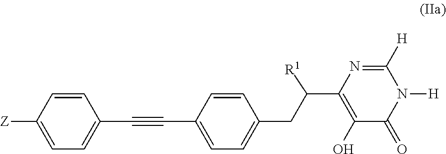

wherein,
R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹);
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl.

One embodiment provides a compound, or a pharmaceutically acceptable salt thereof, having the structure of Formula (IIIa):

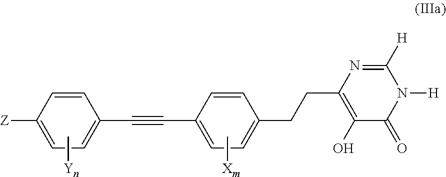

wherein,
n is 1 or 2 and m is 0, 1, or 2; or n is 0, 1, or 2 and m is 1 or 2;
X is halogen or optionally substituted C₁-C₃ alkyl;
Y is halogen or optionally substituted C₁-C₃ alkyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl.

One embodiment provides a pharmaceutical composition comprising a compound of Formula (I), (Ia), (IIa) or (IIIa), or a pharmaceutically acceptable salt thereof, and a pharmaceutically acceptable excipient.

One embodiment provides a method of treating a gram-negative bacterial infection in a patient in need thereof comprising administering to the patient a pharmaceutical composition comprising a compound of Formula (I), (Ia), (IIa) or (IIIa), or a pharmaceutically acceptable salt thereof, and a pharmaceutically acceptable excipient. In some embodiments provided herein, the gram-negative bacterial infection is selected from pneumonia, sepsis, cystic fibrosis, intra-abdominal infection, skin infection and urinary tract infection. In some embodiments provided herein, the gram-negative bacterial infection is selected from chronic urinary tract infection, complicated urinary tract infection, cystitis, pyelonephritis, urethritis, recurrent urinary tract infections, bladder infections, urethral infections and kidney infections.

One embodiment provides a method of inhibiting UDP-{3-O—[(R)-3-hydroxymyristoyl]}-N-acetylglucosamine deacetylase enzyme comprising contacting the enzyme with a compound of Formula (I), (Ia), (IIa) or (IIIa).

One embodiment provides a method for treating bacterial infection in a patient in need thereof comprising administering to the patient a composition comprising a compound of Formula (I), (Ia), (IIa) or (IIIa), or a pharmaceutically acceptable salt thereof, and a pharmaceutically acceptable excipient.

### INCORPORATION BY REFERENCE

All publications, patents, and patent applications mentioned in this specification are herein incorporated by reference for the specific purposes identified herein.

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH2 radical.

“Cyano” refers to the —CN radical.

“Nitro” refers to the —NO2 radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” or “alkoxyl” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atom (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbomyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Carbocyclylalkynyl” refers to a radical of the formula —R^{c}-carbocyclyl where R^{c} is an alkynylene chain as defined above. The alkynylene chain and the carbocyclyl radical are optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical are optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

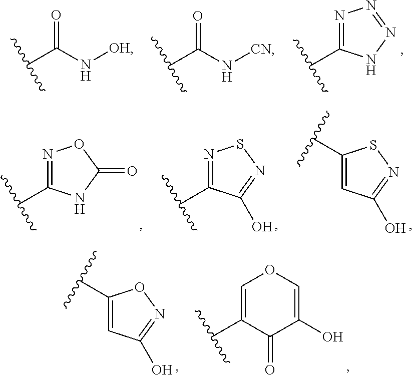

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b-}N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hickel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b-}N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

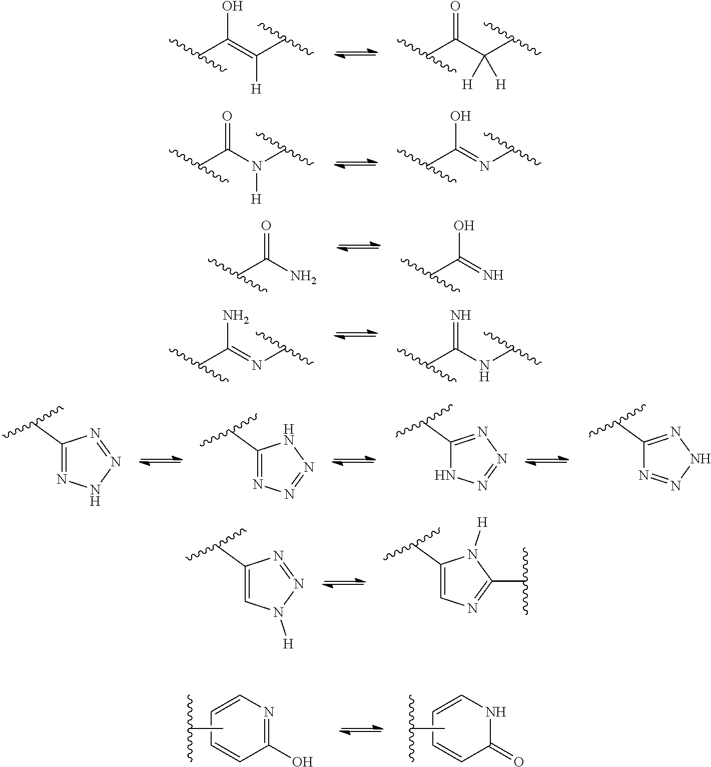

In some instances, the heterocyclic LpxC inhibitory compounds disclosed herein exist in tautomeric forms. The structures of said compounds are illustrated in the one tautomeric form for clarity. The alternative tautomeric forms are expressly included in this disclosure, such as, for example, the structures illustrated below.

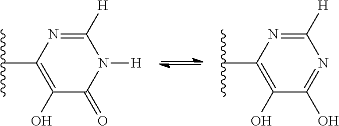 [SMILES: *c1nc[nH]c(=O)c1O ; *c1ncnc(O)c1O]

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes

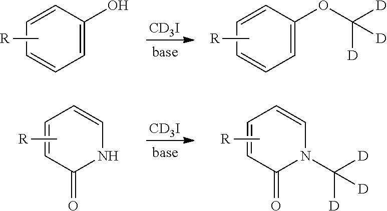

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

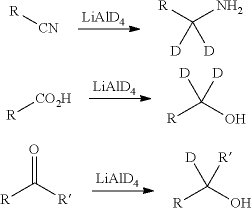

Deuterium gas and palladium catalyst are employed to reduce unsaturated carbon-carbon linkages and to perform a reductive substitution of aryl carbon-halogen bonds as illustrated, by way of example only, in the reaction schemes below.

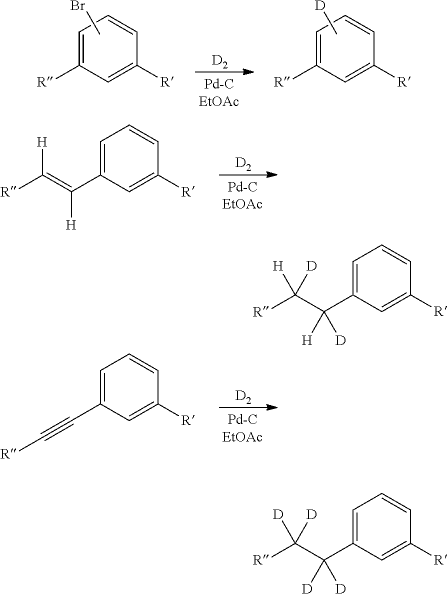

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

“Pharmaceutically acceptable salt” includes both acid and base addition salts. A pharmaceutically acceptable salt of any one of the heterocyclic LpxC inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

“Prodrug” is meant to indicate a compound that is, in some embodiments, converted under physiological conditions or by solvolysis to a biologically active compound described herein. Thus, the term “prodrug” refers to a precursor of a biologically active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

### LpxC Inhibitory Compounds

Provided herein are heterocyclic LpxC inhibitory compounds and pharmaceutical compositions comprising said compounds. The subject compounds and compositions are useful for inhibiting UDP-{3-O—[(R)-3-hydroxymyristoyl]}-N-acetylglucosamine deacetylase (LpxC) and for the treatment of bacterial infection.

One embodiment provides a compound, or a pharmaceutically acceptable salt thereof, having the structure of Formula (I):

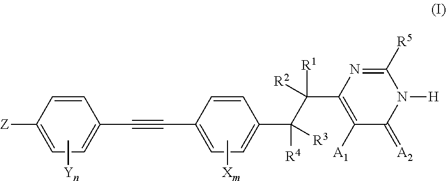

wherein,
n is 0-4;
m is 0-4;
A₁ is OH or SH;
A₂ is O or S;
R¹, R², R³ and R⁴ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹);
or R¹ and R² are taken together to form an optionally substituted alkenyl, ═NR¹¹, oxo, or thioxo;
or R¹ and R² are joined to form an optionally substituted carbocyclyl or optionally substituted heterocyclyl;
or R³ and R⁴ are taken together to form an optionally substituted alkenyl; ═NR¹¹, oxo, or thioxo;
or R³ and R⁴ are joined to form an optionally substituted carbocyclyl or optionally substituted heterocyclyl;
R⁵ is H, halogen, optionally substituted alkyl, hydroxyl, alkoxyl, cyano, amino, or nitro;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2;
and wherein if R¹ is unsubstituted (C₁-C₄ alkylene)-OH, then (i) at least one of R², R³, or R⁴ is not H; or (ii) n is 1-4 and m is 0-4; or (iii) n is 0-4 and m is 1-4;
and wherein if R² is unsubstituted (C₁-C₄ alkylene)-OH, then (i) at least one of R¹, R³, or R⁴ is not H; or (ii) n is 1-4 and m is 0-4; or (iii) n is 0-4 and m is 1-4.

In one embodiment, provided herein is a compound, or a pharmaceutically acceptable salt thereof, of Formula (I):
wherein,
n is 0-4;
m is 0-4;
A₁ is OH or SH
A₂ is O or S;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂R¹¹, optionally substituted (C₁-C₄ alkylene)-SO₂N(R¹¹)₂, optionally substituted (C₁-C₄ alkylene)-SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
R⁵ is H, halogen, optionally substituted alkyl, hydroxyl, alkoxyl, cyano, amino, or nitro;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl.

In one embodiment, provided herein is a compound, or a pharmaceutically acceptable salt thereof, of Formula (I):
wherein,
n is 0-4;
m is 0-4;
A₁ is OH or SH
A₂ is O or S;
R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂R¹¹, optionally substituted (C₁-C₄ alkylene)-SO₂N(R¹¹)₂, optionally substituted (C₁-C₄ alkylene)-SO₂R¹¹, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo;
R⁵ is H, halogen, optionally substituted alkyl, hydroxyl, alkoxyl, cyano, amino, or nitro;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2;
and wherein R¹ and R² are each not a hydroxy substituted alkyl.

In one embodiment, provided herein is a compound, or a pharmaceutically acceptable salt thereof, of Formula (I) wherein R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹).

In one embodiment, provided herein is a compound, or a pharmaceutically acceptable salt thereof, of Formula (I) wherein R¹, R², R³ and R⁴ are each independently H, optionally substituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂.

In one embodiment, provided herein is a compound, or a pharmaceutically acceptable salt thereof, of Formula (I) wherein R¹, R², R³ and R⁴ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂.

In one embodiment, provided herein is a compound, or a pharmaceutically acceptable salt thereof, of Formula (I) wherein if R¹, R², R³, and R⁴ are H, then n is 1 or 2 and m is 0, 1, or 2; or n is 0, 1, or 2 and m is 1 or 2.

In one embodiment, provided herein is a compound, or a pharmaceutically acceptable salt thereof, of Formula (I) and wherein if R¹ is unsubstituted (C₁-C₄ alkylene)-OH, then (i) at least one of R², R³, or R⁴ is not H; or (ii) n is 1-4 and m is 0-4; or (iii) n is 0-4 and m is 1-4. In one embodiment, provided herein is a compound, or a pharmaceutically acceptable salt thereof, of Formula (I) and wherein if R¹ is unsubstituted (C₁-C₄ alkylene)-OH, then at least one of R², R³, or R⁴ is not H. In one embodiment, provided herein is a compound, or a pharmaceutically acceptable salt thereof, of Formula (I) and wherein if R¹ is unsubstituted (C₁-C₄ alkylene)-OH, then n is 1-4 and m is 0-4. In one embodiment, provided herein is a compound, or a pharmaceutically acceptable salt thereof, of Formula (I) and wherein if R¹ is unsubstituted (C₁-C₄ alkylene)-OH, then n is 0-4 and m is 1-4.

In one embodiment, provided herein is a compound, or a pharmaceutically acceptable salt thereof, of Formula (I) and wherein if R² is unsubstituted (C₁-C₄ alkylene)-OH, then (i) at least one of R¹, R³, or R⁴ is not H; or (ii) n is 1-4 and m is 0-4; or (iii) n is 0-4 and m is 1-4. In one embodiment, provided herein is a compound, or a pharmaceutically acceptable salt thereof, of Formula (I) and wherein if R² is unsubstituted (C₁-C₄ alkylene)-OH, then at least one of R¹, R³, or R⁴ is not H. In one embodiment, provided herein is a compound, or a pharmaceutically acceptable salt thereof, of Formula (I) and wherein if R² is unsubstituted (C₁-C₄ alkylene)-OH, then n is 1-4 and m is 0-4. In one embodiment, provided herein is a compound, or a pharmaceutically acceptable salt thereof, of Formula (I) and wherein if R² is unsubstituted (C₁-C₄ alkylene)-OH, then n is 0-4 and m is 1-4.

In one embodiment, provided herein is a compound, or a pharmaceutically acceptable salt thereof, of Formula (I) wherein R¹ is not a hydroxy substituted alkyl. One embodiment provides for a compound, or a pharmaceutically acceptable salt thereof, of Formula (I) wherein R² is not a hydroxy substituted alkyl. In one embodiment, provided herein is a compound, or a pharmaceutically acceptable salt thereof, of Formula (I) wherein R¹ is not a hydroxy substituted alkyl; and R² is not a hydroxy substituted alkyl. One embodiment provides for a compound, or a pharmaceutically acceptable salt thereof, of Formula (Ia) wherein R¹ and R² are each not a hydroxy substituted alkyl.

In one embodiment, provided herein is a compound, or a pharmaceutically acceptable salt thereof, of Formula (I) wherein the compound is not 5-hydroxy-6-(3-hydroxy-3-methyl-1-(4-((4-(morpholinomethyl)phenyl)ethynyl)phenyl)butan-2-yl)pyrimidin-4(3H)-one. In one embodiment, provided herein is a compound of Formula (I), wherein the compound is not 5-hydroxy-6-(3-hydroxy-1-(4-((4-(morpholinomethyl)phenyl)ethynyl)phenyl)butan-2-yl)pyrimidin-4(3H)-one. In one embodiment, provided herein is a compound of Formula (I), wherein the compound is not 5-hydroxy-6-(1-hydroxy-3-(4-((4-(morpholinomethyl)phenyl)ethynyl)phenyl)propan-2-yl)pyrimidin-4(3H)-one. In one embodiment, provided herein is a compound of Formula (I), wherein the compound is not 5-hydroxy-6-(4-((4-(morpholinomethyl)phenyl)ethynyl)phenethyl)pyrimidin-4(3H)-one.

One embodiment provides a compound, or a pharmaceutically acceptable salt thereof, having the structure of Formula (Ia):

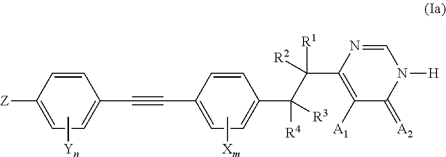

wherein,
n is 0, 1, or 2;
m is 0, 1, or 2;
A₁ is OH or SH;
A₂ is O or S;
R¹ and R³ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹);
R² is H or optionally substituted C₁-C₃ alkyl, provided that if R¹ is H, then R² is H or unsubstituted C₁-C₃ alkyl; or R¹ and R² are taken together to form an optionally substituted alkenyl; or R¹ and R² are joined to form an optionally substituted carbocyclyl or optionally substituted heterocyclyl;
R⁴ is H or optionally substituted C₁-C₃ alkyl; or R³ and R⁴ are taken together to form an optionally substituted alkenyl; or R³ and R⁴ are joined to form an optionally substituted carbocyclyl or optionally substituted heterocyclyl;
X is halogen, hydroxy, or optionally substituted C₁-C₃ alkyl;
Y is halogen, hydroxy, or optionally substituted C₁-C₃ alkyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2;
and wherein if R¹ is unsubstituted (C₁-C₄ alkylene)-OH, then (i) at least one of R², R³, or R⁴ is not H; or (ii) n is 1 or 2 and m is 0, 1, or 2; or (iii) n is 0, 1, or 2 and m is 1 or 2;
and wherein if R² is unsubstituted (C₁-C₄ alkylene)-OH, then (i) at least one of R¹, R³, or R⁴ is not H; or (ii) n is 1 or 2 and m is 0, 1, or 2; or (iii) n is 0, 1, or 2 and m is 1 or 2.

One embodiment provides a compound, or a pharmaceutically acceptable salt thereof, having the structure of Formula (Ia):
wherein,
n is 0, 1, or 2;
m is 0, 1, or 2;
A₁ is OH or SH;
A₂ is O or S;
R¹ and R³ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl, provided that if R¹ is H, then R² is H or unsubstituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
X is halogen, hydroxy, or optionally substituted C₁-C₃ alkyl;
Y is halogen, hydroxy, or optionally substituted C₁-C₃ alkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2.

One embodiment provides a compound, or a pharmaceutically acceptable salt thereof, having the structure of Formula (Ia):
wherein,
n is 0, 1, or 2;
m is 0, 1, or 2;
A₁ is OH or SH;
A₂ is O or S;
R¹ and R³ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl, provided that if R¹ is H, then R² is H or unsubstituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
X is halogen, hydroxy, or optionally substituted C₁-C₃ alkyl;
Y is halogen, hydroxy, or optionally substituted C₁-C₃ alkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2;
and wherein R¹ and R² are each not a hydroxy substituted alkyl.

One embodiment provides a compound, or a pharmaceutically acceptable salt thereof, having the structure of Formula (Ia):
wherein,
n is 0 or 1;
m is 0 or 1;
A₁ is OH;
A₂ is O;
R¹ and R³ are each independently H, alkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, or optionally substituted (C₁-C₄ alkylene)N(R¹²)—SO₂N(R¹¹)₂, optionally substituted (C₁-C₄ alkylene)-O—SO₂N(R¹¹)₂;
R² is H or optionally substituted C₁-C₃ alkyl, provided that if R¹ is H, then R² is H or unsubstituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
X is halogen;
Y is halogen;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹¹ is independently selected from H, alkyl, fluoroalkyl, alkenyl, or carbocyclyl; or two R¹¹ groups together with the nitrogen to which they are attached join to form an optionally substituted N-heterocyclyl;
each R¹² is independently selected from H or alkyl;
each R¹³ is independently selected from H, alkyl, carbocyclyl, carbocyclylalkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 and m is 0 or 1; or n is 0 or 1 and m is 1;
and wherein R¹ is not hydroxy substituted alkyl.

One embodiment provides a compound, or a pharmaceutically acceptable salt thereof, having the structure of Formula (Ia):
wherein,
n is 0 or 1;
m is 0 or 1;
A₁ is OH;
A₂ is O;
R¹ and R³ are each independently H, alkyl, —COR¹¹, —CON(R¹¹)₂, (C₁-C₄ alkylene)-CN, (C₁-C₄ alkylene)-OR¹¹, (C₁-C₄ alkylene)-N(R¹¹)₂, (C₁-C₄ alkylene)N(R¹²)—COR¹¹, (C₁-C₄ alkylene)N(R¹²)—CO₂R¹¹, (C₁-C₄ alkylene)N(R¹²)—CON(R¹¹)₂, or (C₁-C₄ alkylene)N(R¹²)—SO₂N(R¹¹)₂, (C₁-C₄ alkylene)-O—SO₂N(R¹¹)₂;
R² is H or C₁-C₃ alkyl, provided that if R¹ is H, then R² is H or unsubstituted C₁-C₃ alkyl; or R¹ and R² are taken together to form an optionally substituted alkenyl;
R⁴ is H or C₁-C₃ alkyl; or R³ and R⁴ are taken together to form an alkenyl;
X is halogen;
Y is halogen;
Z is H, -L-G, (C₁-C₄ alkylene)-OCON(R¹³)₂, (C₁-C₄ alkylene)-N(R¹⁴)CON(R¹³)₂, or (C₁-C₄ alkylene)-SO₂N(R¹³)₂, —SO₂(R¹³);
L is a bond, C₁-C₄ alkylene, —C(O)—, or —SO₂—;
G is alkyl, heterocyclyl, heteroaryl, —N(R¹³)₂, —OR¹³, or —CN;
each R¹¹ is independently selected from H, alkyl, fluoroalkyl, alkenyl, or carbocyclyl; or two R¹¹ groups together with the nitrogen to which they are attached join to form an optionally substituted N-heterocyclyl;
each R¹² is independently selected from H or alkyl;
each R¹³ is independently selected from H, alkyl, carbocyclyl, carbocyclylalkyl, heterocyclyl, heterocyclylalkyl, heteroaryl, or heteroarylalkyl; or two R¹³ groups together with the nitrogen to which they are attached join to form an optionally substituted N-heterocyclyl; and
each R¹⁴ is independently selected from H or alkyl;
wherein if R¹, R², R³, and R⁴ are H, then n is 1 and m is 0 or 1; or n is 0 or 1 and m is 1;
and wherein R¹ is not hydroxy substituted alkyl.

One embodiment provides a compound, or a pharmaceutically acceptable salt thereof, having the structure of Formula (Ia):
wherein,
n is 0 or 1;
m is 0 or 1;
A₁ is OH;
A₂ is O;
R¹ is alkyl, —COR¹¹, —CON(R¹¹)₂, (C₁-C₄ alkylene)-CN, (C₁-C₄ alkylene)-OR¹¹, (C₁-C₄ alkylene)-N(R¹¹)₂, (C₁-C₄ alkylene)N(R¹²)—COR¹¹, (C₁-C₄ alkylene)N(R¹²)—CO₂R, (C₁-C₄ alkylene)N(R¹²)—CON(R¹¹)₂, or (C₁-C₄ alkylene)N(R¹²)—SO₂N(R¹¹)₂, (C₁-C₄ alkylene)-O—SO₂N(R¹¹)₂;
R² is H; or R¹ and R² are taken together to form an alkenyl;
R³ is H;
R⁴ is H;
X is halogen;
Y is halogen;
Z is H, -L-G, (C₁-C₄ alkylene)-OCON(R¹³)₂, (C₁-C₄ alkylene)-N(R¹⁴)CON(R¹³)₂, or (C₁-C₄ alkylene)-SO₂N(R¹³)₂, —SO₂(R¹³);
L is a bond, C₁-C₄ alkylene, —C(O)—, or —SO₂—;
G is alkyl, heterocyclyl, heteroaryl, —N(R¹³)₂, —OR¹³, or —CN;
each R¹¹ is independently selected from H, alkyl, fluoroalkyl, alkenyl, or carbocyclyl; or two R¹¹ groups together with the nitrogen to which they are attached join to form an optionally substituted N-heterocyclyl;
each R¹² is independently selected from H or alkyl;
each R¹³ is independently selected from H, alkyl, carbocyclyl, carbocyclylalkyl, heterocyclyl, heterocyclylalkyl, heteroaryl, or heteroarylalkyl; or two R¹³ groups together with the nitrogen to which they are attached join to form an optionally substituted N-heterocyclyl; and
each R¹⁴ is independently selected from H or alkyl;
wherein R¹ is not hydroxy substituted alkyl.

One embodiment provides a compound, or a pharmaceutically acceptable salt thereof, having the structure of Formula (Ia): wherein,
n is 0 or 1;
m is 0 or 1;
A₁ is OH;
A₂ is O;
R¹ is alkyl, —COR¹¹, —CON(R¹¹)₂, (C₁-C₄ alkylene)-CN, (C₁-C₄ alkylene)-OR¹¹, (C₁-C₄ alkylene)-N(R¹¹)₂, (C₁-C₄ alkylene)N(R¹²)—COR¹¹, (C₁-C₄ alkylene)N(R¹²)—CO₂R¹¹;
R² is H; or R¹ and R² are taken together to form an alkenyl;
R³ is H;
R⁴ is H;
X is halogen;
Y is halogen;
Z is H, -L-G, (C₁-C₄ alkylene)-OCON(R¹³)₂, (C₁-C₄ alkylene)-N(R¹⁴)CON(R¹³)₂, or (C₁-C₄ alkylene)-SO₂N(R¹³)₂, —SO₂(R¹³);
L is a bond, C₁-C₄ alkylene, —C(O)—, or —SO₂—;
G is alkyl, heterocyclyl, heteroaryl, —N(R¹³)₂, —OR¹³, or —CN;
each R¹¹ is independently selected from H, alkyl, fluoroalkyl, or alkenyl;
each R¹² is independently selected from H or alkyl;
each R¹³ is independently selected from H, alkyl, carbocyclyl, carbocyclylalkyl, heterocyclyl, heterocyclylalkyl; or two R¹³ groups together with the nitrogen to which they are attached join to form an optionally substituted N-heterocyclyl; and
each R¹⁴ is independently selected from H or alkyl;
wherein R¹ is not hydroxy substituted alkyl.

One embodiment provides a compound, or a pharmaceutically acceptable salt thereof, having the structure of Formula (Ia):
wherein,
n is 0 or 1;
m is 1;
A₁ is OH;
A₂ is O;
R¹ is H, alkyl, —COR¹¹, —CON(R¹¹)₂, (C₁-C₄ alkylene)-CN, (C₁-C₄ alkylene)-OR¹, (C₁-C₄ alkylene)-N(R¹¹)₂, (C₁-C₄ alkylene)N(R¹²)—COR¹¹, (C₁-C₄ alkylene)N(R¹²)—CO₂R¹¹, (C₁-C₄ alkylene)N(R¹²)—CON(R′)₂, or (C₁-C₄ alkylene)N(R¹²)—SO₂N(R¹¹)₂, (C₁-C₄ alkylene)-O—SO₂N(R¹¹)₂;
R² is H; or R¹ and R² are taken together to form an alkenyl;
R³ is H;
R⁴ is H;
X is halogen;
Y is halogen;
Z is H, -L-G, (C₁-C₄ alkylene)-OCON(R¹³)₂, (C₁-C₄ alkylene)-N(R¹⁴)CON(R¹³)₂, or (C₁-C₄ alkylene)-SO₂N(R¹³)₂, —SO₂(R¹³);
L is a bond, C₁-C₄ alkylene, —C(O)—, or —SO₂—;
G is alkyl, heterocyclyl, heteroaryl, —N(R¹³)₂, —OR¹³, or —CN;
each R¹¹ is independently selected from H, alkyl, fluoroalkyl, alkenyl, or carbocyclyl; or two R¹¹ groups together with the nitrogen to which they are attached join to form an optionally substituted N-heterocyclyl;
each R¹² is independently selected from H or alkyl;
each R¹³ is independently selected from H, alkyl, carbocyclyl, carbocyclylalkyl, heterocyclyl, heterocyclylalkyl, heteroaryl, or heteroarylalkyl; or two R¹³ groups together with the nitrogen to which they are attached join to form an optionally substituted N-heterocyclyl; and
each R¹⁴ is independently selected from H or alkyl.

One embodiment provides a compound, or a pharmaceutically acceptable salt thereof, having the structure of Formula (Ia):
wherein,
n is 0 or 1;
m is 1;
A₁ is OH;
A₂ is O;
R¹ is H, alkyl, —COR¹¹, —CON(R¹¹)₂, (C₁-C₄ alkylene)-CN, (C₁-C₄ alkylene)-OR¹¹, (C₁-C₄ alkylene)-N(R¹¹)₂, (C₁-C₄ alkylene)N(R¹²)—COR¹¹, (C₁-C₄ alkylene)N(R¹²)—CO₂R¹¹, (C₁-C₄ alkylene)N(R¹²)—CON(R¹¹)₂, or (C₁-C₄ alkylene)N(R¹²)—SO₂N(R¹¹)₂, (C₁-C₄ alkylene)-O—SO₂N(R¹¹)₂;
R² is H; or R¹ and R² are taken together to form an alkenyl;
R³ is H;
R⁴ is H;
X is halogen;
Y is halogen;
Z is H, -L-G, (C₁-C₄ alkylene)-OCON(R¹³)₂, (C₁-C₄ alkylene)-N(R¹⁴)CON(R¹³)₂, or (C₁-C₄ alkylene)-SO₂N(R¹³)₂, —SO₂(R¹³);
L is a bond, C₁-C₄ alkylene, —C(O)—, or —SO₂—;
G is alkyl, heterocyclyl, heteroaryl, —N(R¹³)₂, —OR¹³, or —CN;
each R¹¹ is independently selected from H, alkyl, fluoroalkyl, or alkenyl;
each R¹² is independently selected from H or alkyl;
each R¹³ is independently selected from H, alkyl, carbocyclyl, carbocyclylalkyl, heterocyclyl, or heterocyclylalkyl; or two R¹³ groups together with the nitrogen to which they are attached join to form an optionally substituted N-heterocyclyl; and
each R¹⁴ is independently selected from H or alkyl.

One embodiment provides for a compound, or a pharmaceutically acceptable salt thereof, of Formula (Ia) wherein A1 is OH. One embodiment provides for a compound, or a pharmaceutically acceptable salt thereof, of Formula (Ia) wherein A2 is O. One embodiment provides for a compound, or a pharmaceutically acceptable salt thereof, of Formula (Ia) wherein A1 is OH and A2 is O.

One embodiment provides for a compound, or a pharmaceutically acceptable salt thereof, of Formula (Ia) wherein R1 is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR11, —CON(R11)₂, optionally substituted (C₁-C₄ alkylene)-CN, optionally substituted (C1-C4 alkylene)-OR11, optionally substituted (C1-C4 alkylene)-N(R11)2, optionally substituted (C1-C4 alkylene)N(R12)-COR11, optionally substituted (C1-C4 alkylene)N(R12)-CO2R11, optionally substituted (C1-C4 alkylene)N(R12)-CON(R11)2, optionally substituted (C1-C4 alkylene)N(R12)-SO2R11, optionally substituted (C1-C4 alkylene)-SO2N(R11)2, optionally substituted (C1-C4 alkylene)-SO2R11, optionally substituted (C1-C4 alkylene)N(R12)-SO2N(R11)2, optionally substituted (C1-C4 alkylene)-O—SO2N(R11)2, optionally substituted (C1-C4 alkylene)N(R11)-PO (optionally substituted C1-C4 alkyl)2, optionally substituted (C1-C4 alkylene)-SO2R11, optionally substituted (C1-C4 alkylene)-O—SO2R11, or optionally substituted (C0-C4 alkylene)-C(═N—OR11)(R11).

One embodiment provides for a compound, or a pharmaceutically acceptable salt thereof, of Formula (Ia) wherein R1 is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR11, —CON(R11)2, optionally substituted (C1-C4 alkylene)-CN, optionally substituted (C1-C4 alkylene)-N(R11)2, optionally substituted (C1-C4 alkylene)N(R12)-COR11, optionally substituted (C1-C4 alkylene)N(R12)-CO2R11, optionally substituted (C1-C4 alkylene)N(R12)-CON(R11)2, optionally substituted (C1-C4 alkylene)N(R12)-SO2R11, optionally substituted (C1-C4 alkylene)-SO2N(R11)2, optionally substituted (C1-C4 alkylene)-SO2R11, optionally substituted (C1-C4 alkylene)N(R12)-SO2N(R11)2, optionally substituted (C1-C4 alkylene)-O—SO2N(R11)2, optionally substituted (C1-C4 alkylene)N(R11)-PO (optionally substituted C1-C4 alkyl)2, optionally substituted (C1-C4 alkylene)-SO2R11, optionally substituted (C1-C4 alkylene)-O—SO2R11, or optionally substituted (C0-C4 alkylene)-C(═N—OR11)(R11).

One embodiment provides for a compound, or a pharmaceutically acceptable salt thereof, of Formula (Ia) wherein R1 is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR11, —CON(R11)2, optionally substituted (C1-C4 alkylene)-CN, optionally substituted (C1-C4 alkylene)-OR11, optionally substituted (C1-C4 alkylene)-N(R11)2, optionally substituted (C1-C4 alkylene)N(R12)-COR11, optionally substituted (C1-C4 alkylene)N(R12)-CO2R11, optionally substituted (C1-C4 alkylene)N(R12)-CON(R11)2, optionally substituted (C1-C4 alkylene)N(R12)-SO2R11, optionally substituted (C1-C4 alkylene)-SO2N(R11)2, optionally substituted (C1-C4 alkylene)-SO2R11, optionally substituted (C1-C4 alkylene)N(R12)-SO2N(R11)2, optionally substituted (C1-C4 alkylene)-O—SO2N(R11)2, or optionally substituted (C1-C4 alkylene)N(R11)-PO (optionally substituted C1-C4 alkyl)2.

One embodiment provides for a compound, or a pharmaceutically acceptable salt thereof, of Formula (Ia) wherein R1 is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR11, —CON(R11)2, optionally substituted (C1-C4 alkylene)-CN, optionally substituted (C1-C4 alkylene)-OR11, optionally substituted (C1-C4 alkylene)-N(R11)2, optionally substituted (C1-C4 alkylene)N(R12)-COR11, optionally substituted (C1-C4 alkylene)N(R12)-CO2R11, optionally substituted (C1-C4 alkylene)N(R12)-CON(R11)2, optionally substituted (C1-C4 alkylene)N(R12)-SO2N(R11)2, optionally substituted (C1-C4 alkylene)-O—SO2N(R11)2, or optionally substituted (C1-C4 alkylene)N(R11)-PO (optionally substituted C1-C4 alkyl)2.

One embodiment provides for a compound, or a pharmaceutically acceptable salt thereof, of Formula (Ia) wherein R1 is unsubstituted alkyl, —COR11, —CON(R11)2, optionally substituted (C1-C4 alkylene)-CN, optionally substituted (C1-C4 alkylene)-OR11, optionally substituted (C1-C4 alkylene)-N(R11)2, optionally substituted (C1-C4 alkylene)N(R12)-SO2R11, optionally substituted (C1-C4 alkylene)-SO2N(R11)2, or optionally substituted (C1-C4 alkylene)-SO2R11. One embodiment provides for a compound, or a pharmaceutically acceptable salt thereof, of Formula (Ia) wherein R1 is unsubstituted alkyl, —COR11, —CON(R11)2, (C1-C4 alkylene)-CN, (C1-C4 alkylene)-OR11, (C1-C4 alkylene)-N(R11)2, (C1-C4 alkylene)N(R12)-SO2R11, (C1-C4 alkylene)-SO2N(R11)2, or (C1-C4 alkylene)-SO2R11. One embodiment provides for a compound, or a pharmaceutically acceptable salt thereof, of Formula (Ia) wherein R1 is unsubstituted alkyl, —COR11, —CON(R11)2, (C1 alkylene)-CN, (C1 alkylene)-OR11, (C1 alkylene)-N(R11)2, (C1 alkylene)N(R12)-SO2R11, (C1 alkylene)-SO2N(R11)2, or (C1 alkylene)-SO2R11. One embodiment provides for a compound, or a pharmaceutically acceptable salt thereof, of Formula (Ia) wherein R1 is —COR11, —CON(R11)2, (C1 alkylene)-CN, (C1 alkylene)-OR11, (C1 alkylene)-N(R11)2, (C1 alkylene)N(R12)-SO2R11, (C1 alkylene)-SO2N(R11)2, or (C1 alkylene)-SO2R11.

One embodiment provides for a compound, or a pharmaceutically acceptable salt thereof, of Formula (Ia) wherein R1 is unsubstituted alkyl, —COR11, —CON(R11)2, optionally substituted (C1-C4 alkylene)-N(R11)2, optionally substituted (C1-C4 alkylene)N(R12)-COR11, optionally substituted (C1-C4 alkylene)N(R12)-CO2R11, or optionally substituted (C1-C4 alkylene)N(R12)-CON(R11)2. One embodiment provides for a compound, or a pharmaceutically acceptable salt thereof, of Formula (Ia) wherein R1 is unsubstituted alkyl, —COR11, —CON(R11)2, (C1-C4 alkylene)-N(R11)2, (C1-C4 alkylene)N(R12)-COR11, (C1-C4 alkylene)N(R12)-CO2R11, or (C1-C4 alkylene)N(R12)-CON(R11)2. One embodiment provides for a compound, or a pharmaceutically acceptable salt thereof, of Formula (Ia) wherein R1 is unsubstituted alkyl, —COR11, —CON(R11)2, (C1-C2 alkylene)-N(R11)2, (C1-C2 alkylene)N(R12)-COR11, (C1-C2 alkylene)N(R12)-CO2R11, or (C1-C2 alkylene)N(R12)-CON(R11)2. One embodiment provides for a compound, or a pharmaceutically acceptable salt thereof, of Formula (Ia) wherein R1 is —COR11, —CON(R11)2, (C1 alkylene)-N(R11)2, (C1 alkylene)N(R12)-COR11, (C1 alkylene)N(R12)-CO2R11, or (C1 alkylene)N(R12)-CON(R11)2. One embodiment provides for a compound, or a pharmaceutically acceptable salt thereof, of Formula (Ia) wherein R1 is —COR11, —CON(R11)2, (C1-C4 alkylene)-N(R11)2, or (C1-C4 alkylene)N(R12)-CO2R11. One embodiment provides for a compound, or a pharmaceutically acceptable salt thereof, of Formula (Ia) wherein R1 is —COR11, —CON(R11)2, (C1 alkylene)-N(R11)2, or (C1 alkylene)N(R12)-CO2R11.

One embodiment provides for a compound, or a pharmaceutically acceptable salt thereof, of Formula (Ia) wherein R1 is —COR11, —CON(R11)2, optionally substituted (C1-C4 alkylene)-N(R11)2, or optionally substituted (C1-C4 alkylene)N(R12)-COR11.

One embodiment provides for a compound, or a pharmaceutically acceptable salt thereof, of Formula (Ia) wherein R1 is —COR11. One embodiment provides for a compound, or a pharmaceutically acceptable salt thereof, of Formula (Ia) wherein R1 is —CON(R11)2. One embodiment provides for a compound, or a pharmaceutically acceptable salt thereof, of Formula (Ia) wherein R1 is optionally substituted (C1-C4 alkylene)-N(R11)2. One embodiment provides for a compound, or a pharmaceutically acceptable salt thereof, of Formula (Ia) wherein R1 is optionally substituted (C1-C4 alkylene)N(R12)-COR11. One embodiment provides for a compound, or a pharmaceutically acceptable salt thereof, of Formula (Ia) wherein optionally substituted (C1-C4 alkylene) is —CH2-.

One embodiment provides for a compound, or a pharmaceutically acceptable salt thereof, of Formula (Ia) wherein each R11 is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R11 groups together with the nitrogen to which they are attached join to form an optionally substituted N-heterocyclyl. One embodiment provides for a compound, or a pharmaceutically acceptable salt thereof, of Formula (Ia) wherein each R11 is independently H, optionally substituted alkyl, optionally substituted alkenyl, optionally substituted carbocyclyl, optionally substituted heterocyclyl; or two R11 groups together with the nitrogen to which they are attached join to form an optionally substituted N-heterocyclyl.

One embodiment provides for a compound, or a pharmaceutically acceptable salt thereof, of Formula (Ia) wherein R11 is H. One embodiment provides for a compound, or a pharmaceutically acceptable salt thereof, of Formula (Ia) wherein R11 is optionally substituted alkyl. One embodiment provides for a compound, or a pharmaceutically acceptable salt thereof, of Formula (Ia) wherein R11 is hydroxy substituted alkyl. One embodiment provides for a compound, or a pharmaceutically acceptable salt thereof, of Formula (Ia) wherein R11 is fluoro substituted alkyl. One embodiment provides for a compound, or a pharmaceutically acceptable salt thereof, of Formula (Ia) wherein R11 is 2-fluoroethyl. One embodiment provides for a compound, or a pharmaceutically acceptable salt thereof, of Formula (Ia) wherein R11 is fluoromethyl. One embodiment provides for a compound, or a pharmaceutically acceptable salt thereof, of Formula (Ia) wherein R11 is difluoro substituted alkyl. One embodiment provides for a compound, or a pharmaceutically acceptable salt thereof, of Formula (Ia) wherein R11 is 2,2-difluoroethyl. One embodiment provides for a compound, or a pharmaceutically acceptable salt thereof, of Formula (Ia) wherein R11 is difluoromethyl. One embodiment provides for a compound, or a pharmaceutically acceptable salt thereof, of Formula (Ia) wherein R11 is trifluoro substituted alkyl. One embodiment provides for a compound, or a pharmaceutically acceptable salt thereof, of Formula (Ia) wherein R11 is 2,2,2-trifluoroethyl. One embodiment provides for a compound, or a pharmaceutically acceptable salt thereof, of Formula (Ia) wherein R11 is trifluoromethyl. One embodiment provides for a compound, or a pharmaceutically acceptable salt thereof, of Formula (Ia) wherein R11 is amino substituted alkyl. One embodiment provides for a compound, or a pharmaceutically acceptable salt thereof, of Formula (Ia) wherein R11 is cyano substituted alkyl. One embodiment provides for a compound, or a pharmaceutically acceptable salt thereof, of Formula (Ia) wherein R11 is optionally substituted alkenyl. One embodiment provides for a compound, or a pharmaceutically acceptable salt thereof, of Formula (Ia) wherein R11 is optionally substituted carbocyclyl. One embodiment provides for a compound, or a pharmaceutically acceptable salt thereof, of Formula (Ia) wherein R11 is optionally substituted carbocyclylalkyl. One embodiment provides for a compound, or a pharmaceutically acceptable salt thereof, of Formula (Ia) wherein R11 is optionally substituted heterocyclyl. One embodiment provides for a compound, or a pharmaceutically acceptable salt thereof, of Formula (Ia) wherein R11 is optionally substituted heterocyclylalkyl. One embodiment provides for a compound, or a pharmaceutically acceptable salt thereof, of Formula (Ia) wherein R11 is optionally substituted heteroaryl. One embodiment provides for a compound, or a pharmaceutically acceptable salt thereof, of Formula (Ia) wherein R11 is optionally substituted heteroarylalkyl. One embodiment provides for a compound, or a pharmaceutically acceptable salt thereof, of Formula (Ia) wherein two R11 groups together with the nitrogen to which they are attached join to form an optionally substituted N-heterocyclyl. One embodiment provides for a compound, or a pharmaceutically acceptable salt thereof, of Formula (Ia) wherein R11 is vinyl, propan-2-yl, methyl, ethyl, cyclopropyl, cyclopentyl, azentidin-1-yl or allyl.

One embodiment provides for a compound, or a pharmaceutically acceptable salt thereof, of Formula (Ia) wherein R1 and R2 are joined to form an optionally substituted carbocyclyl or optionally substituted heterocyclyl. One embodiment provides for a compound, or a pharmaceutically acceptable salt thereof, of Formula (Ia) wherein R1 and R2 are joined to form an optionally substituted carbocyclyl. One embodiment provides for a compound, or a pharmaceutically acceptable salt thereof, of Formula (Ia) wherein R1 and R2 are joined to form an optionally substituted heterocyclyl. One embodiment provides for a compound, or a pharmaceutically acceptable salt thereof, of Formula (Ia) wherein R1 and R2 are joined to form a cyclopropyl.

One embodiment provides for a compound, or a pharmaceutically acceptable salt thereof, of Formula (Ia) wherein R2 is H. One embodiment provides for a compound, or a pharmaceutically acceptable salt thereof, of Formula (Ia) wherein R3 is H. One embodiment provides for a compound, or a pharmaceutically acceptable salt thereof, of Formula (Ia) wherein R4 is H. One embodiment provides for a compound, or a pharmaceutically acceptable salt thereof, of Formula (Ia) wherein n is 0 or 1. One embodiment provides for a compound, or a pharmaceutically acceptable salt thereof, of Formula (Ia) wherein m is 0 or 1. One embodiment provides for a compound, or a pharmaceutically acceptable salt thereof, of Formula (Ia) wherein n is 1 or 2. One embodiment provides for a compound, or a pharmaceutically acceptable salt thereof, of Formula (Ia) wherein m is 1 or 2. One embodiment provides for a compound, or a pharmaceutically acceptable salt thereof, of Formula (Ia) wherein X is halogen. One embodiment provides for a compound, or a pharmaceutically acceptable salt thereof, of Formula (Ia) wherein Y is halogen.

One embodiment provides for a compound, or a pharmaceutically acceptable salt thereof, of Formula (Ia) wherein Z is -L-G, optionally substituted (C1-C4 alkylene)-OCON(R13)2, optionally substituted (C1-C4 alkylene)-N(R14)CON(R13)2, optionally substituted (C1-C4 alkylene)-SO2N(R13)2, or optionally substituted (C1-C4 alkylene)-N(R14)SO2R13. One embodiment provides for a compound, or a pharmaceutically acceptable salt thereof, of Formula (Ia) wherein Z is -L-G, optionally substituted (C1-C4 alkylene)-OCON(R13)2, optionally substituted (C1-C4 alkylene)-N(R14)CON(R13)2, or optionally substituted (C1-C4 alkylene)-SO2N(R13)2.

One embodiment provides for a compound, or a pharmaceutically acceptable salt thereof, of Formula (Ia) wherein Z is -L-G.

One embodiment provides for a compound, or a pharmaceutically acceptable salt thereof, of Formula (Ia) wherein Z is morpholinomethyl, (8-oxa-3-azabicyclo[3.2.1]octan-3-yl)methyl, (2-oxa-5-azabicyclo[2.2.1]heptan-5-yl)methyl, (1,1-dioxidothiomorpholino)methyl, (oxetan-3-ylamino)methyl, ((methoxycyclobutyl)amino)methyl, (methylpiperazin-1-yl)methyl, (cyanopyrrolidin-1-yl)methyl, or (methoxypyrrolidin-1-yl)methyl.

One embodiment provides for a compound, or a pharmaceutically acceptable salt thereof, of Formula (Ia) wherein L is a bond or optionally substituted C1-C4 alkylene. One embodiment provides for a compound, or a pharmaceutically acceptable salt thereof, of Formula (Ia) wherein L is a bond. One embodiment provides for a compound, or a pharmaceutically acceptable salt thereof, of Formula (Ia) wherein L is an optionally substituted C1-C4 alkylene. One embodiment provides for a compound, or a pharmaceutically acceptable salt thereof, of Formula (Ia) wherein L is optionally substituted C1. One embodiment provides for a compound, or a pharmaceutically acceptable salt thereof, of Formula (Ia) wherein L is —CH2-.

One embodiment provides for a compound, or a pharmaceutically acceptable salt thereof, of Formula (Ia) wherein G is optionally substituted alkyl, optionally substituted heterocyclyl, optionally substituted heteroaryl, —N(R13)2, —OR13, or —CN. One embodiment provides for a compound, or a pharmaceutically acceptable salt thereof, of Formula (Ia) wherein G is optionally substituted alkyl. One embodiment provides for a compound, or a pharmaceutically acceptable salt thereof, of Formula (Ia) wherein G is optionally substituted heterocyclyl. One embodiment provides for a compound, or a pharmaceutically acceptable salt thereof, of Formula (Ia) wherein G is optionally substituted morpholinyl, piperidinyl, piperazinyl, pyrolidinyl, imidazolyl, imidazolidinyl. One embodiment provides for a compound, or a pharmaceutically acceptable salt thereof, of Formula (Ia) wherein G is optionally substituted morpholinyl. One embodiment provides for a compound, or a pharmaceutically acceptable salt thereof, of Formula (Ia) wherein G is unsubstituted morpholinyl. One embodiment provides for a compound, or a pharmaceutically acceptable salt thereof, of Formula (Ia) wherein G is optionally substituted heteroaryl. One embodiment provides for a compound, or a pharmaceutically acceptable salt thereof, of Formula (Ia) wherein G is —N(R13)2. One embodiment provides for a compound, or a pharmaceutically acceptable salt thereof, of Formula (Ia) wherein G is —OR13. One embodiment provides for a compound, or a pharmaceutically acceptable salt thereof, of Formula (Ia) wherein G is —CN.

One embodiment provides for a compound, or a pharmaceutically acceptable salt thereof, of Formula (Ia) wherein each R13 is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R13 groups together with the nitrogen to which they are attached join to form an optionally substituted N-heterocyclyl.

One embodiment provides for a compound, or a pharmaceutically acceptable salt thereof, of Formula (Ia) wherein R13 is H. One embodiment provides for a compound, or a pharmaceutically acceptable salt thereof, of Formula (Ia) wherein R13 is optionally substituted alkyl. One embodiment provides for a compound, or a pharmaceutically acceptable salt thereof, of Formula (Ia) wherein R13 is optionally substituted carbocyclyl. One embodiment provides for a compound, or a pharmaceutically acceptable salt thereof, of Formula (Ia) wherein R13 is optionally substituted carbocyclylalkyl. One embodiment provides for a compound, or a pharmaceutically acceptable salt thereof, of Formula (Ia) wherein R13 is optionally substituted heterocyclyl. One embodiment provides for a compound, or a pharmaceutically acceptable salt thereof, of Formula (Ia) wherein R13 is optionally substituted heterocyclylalkyl. One embodiment provides for a compound, or a pharmaceutically acceptable salt thereof, of Formula (Ia) wherein R13 is optionally substituted heteroaryl. One embodiment provides for a compound, or a pharmaceutically acceptable salt thereof, of Formula (Ia) wherein R13 is optionally substituted heteroarylalkyl. One embodiment provides for a compound, or a pharmaceutically acceptable salt thereof, of Formula (Ia) wherein two R13 groups together with the nitrogen to which they are attached join to form an optionally substituted N-heterocyclyl.

In one embodiment, provided herein is a compound, or a pharmaceutically acceptable salt thereof, of Formula (Ia) wherein if R1, R2, R3, and R4 are H, then n is 1 or 2 and m is 0, 1, or 2; or n is 0, 1, or 2 and m is 1 or 2.

In one embodiment, provided herein is a compound, or a pharmaceutically acceptable salt thereof, of Formula (Ia) and wherein if R1 is unsubstituted (C1-C4 alkylene)-OH, then (i) at least one of R2, R3, or R4 is not H; or (ii) n is 1 or 2 and m is 0, 1, or 2; or (iii) n is 0, 1, or 2 and m is 1 or 2. In one embodiment, provided herein is a compound, or a pharmaceutically acceptable salt thereof, of Formula (Ia) and wherein if R1 is unsubstituted (C1-C4 alkylene)-OH, then at least one of R2, R3, or R4 is not H. In one embodiment, provided herein is a compound, or a pharmaceutically acceptable salt thereof, of Formula (Ia) and wherein if R1 is unsubstituted (C1-C4 alkylene)-OH, then n is 1 or 2 and m is 0, 1, or 2. In one embodiment, provided herein is a compound, or a pharmaceutically acceptable salt thereof, of Formula (Ia) and wherein if R1 is unsubstituted (C1-C4 alkylene)-OH, then n is 0, 1, or 2 and m is 1 or 2.

In one embodiment, provided herein is a compound, or a pharmaceutically acceptable salt thereof, of Formula (Ia) and wherein if R2 is unsubstituted (C1-C4 alkylene)-OH, then (i) at least one of R1, R3, or R4 is not H; or (ii) n is 1 or 2 and m is 0, 1, or 2; or (iii) n is 0, 1, or 2 and m is 1 or 2. In one embodiment, provided herein is a compound, or a pharmaceutically acceptable salt thereof, of Formula (Ia) and wherein if R2 is unsubstituted (C1-C4 alkylene)-OH, then at least one of R1, R3, or R4 is not H. In one embodiment, provided herein is a compound, or a pharmaceutically acceptable salt thereof, of Formula (Ia) and wherein if R2 is unsubstituted (C1-C4 alkylene)-OH, then n is 1 or 2 and m is 0, 1, or 2. In one embodiment, provided herein is a compound, or a pharmaceutically acceptable salt thereof, of Formula (Ia) and wherein if R2 is unsubstituted (C1-C4 alkylene)-OH, then n is 0, 1, or 2 and m is 1 or 2.

One embodiment provides for a compound, or a pharmaceutically acceptable salt thereof, of Formula (Ia) wherein R1 is not a hydroxy substituted alkyl. One embodiment provides for a compound, or a pharmaceutically acceptable salt thereof, of Formula (Ia) wherein R2 is not a hydroxy substituted alkyl. One embodiment provides for a compound, or a pharmaceutically acceptable salt thereof, of Formula (Ia) wherein R1 is not a hydroxy substituted alkyl; and R2 is not a hydroxy substituted alkyl. One embodiment provides for a compound, or a pharmaceutically acceptable salt thereof, of Formula (Ia) wherein R1 and R2 are each not a hydroxy substituted alkyl.

One embodiment provides for a compound, or a pharmaceutically acceptable salt thereof, of Formula (Ia) wherein the compound is not 5-hydroxy-6-(3-hydroxy-3-methyl-1-(4-((4-(morpholinomethyl)phenyl)ethynyl)phenyl)butan-2-yl)pyrimidin-4(3H)-one. One embodiment provides for a compound, or a pharmaceutically acceptable salt thereof, of Formula (Ia) wherein the compound is not 5-hydroxy-6-(3-hydroxy-1-(4-((4-(morpholinomethyl)phenyl)ethynyl)phenyl)butan-2-yl)pyrimidin-4(3H)-one. One embodiment provides for a compound, or a pharmaceutically acceptable salt thereof, of Formula (Ia) wherein the compound is not 5-hydroxy-6-(1-hydroxy-3-(4-((4-(morpholinomethyl)phenyl)ethynyl)phenyl)propan-2-yl)pyrimidin-4(3H)-one. One embodiment provides for a compound, or a pharmaceutically acceptable salt thereof, of Formula (Ia) wherein the compound is not 5-hydroxy-6-(4-((4-(morpholinomethyl)phenyl)ethynyl)phenethyl)pyrimidin-4(3H)-one.

One embodiment provides a compound, or a pharmaceutically acceptable salt thereof, having the structure of Formula (IIa):

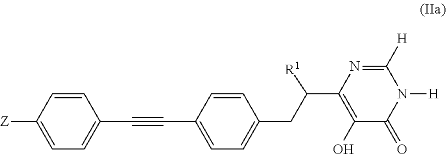

wherein,
R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-N(R¹¹)₂, optionally substituted (C₁-C₄ alkylene)N(R¹²)—COR1, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹);
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl.

One embodiment provides a compound, or a pharmaceutically acceptable salt thereof, having the structure of Formula (IIa):

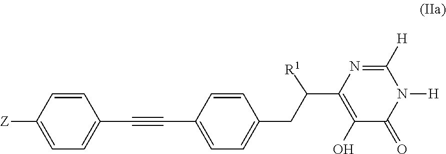

wherein,
R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R′)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl.

One embodiment provides a compound, or a pharmaceutically acceptable salt thereof, having the structure of Formula (IIa):

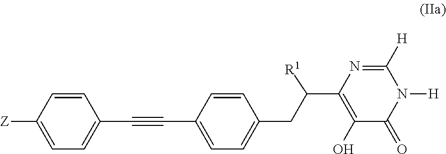

wherein,
R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein R¹ is not a hydroxy substituted alkyl.

One embodiment provides a compound, or a pharmaceutically acceptable salt thereof, having the structure of Formula (IIa):

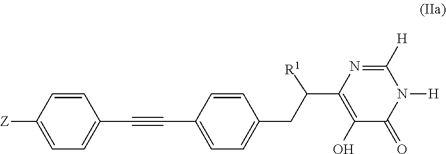

wherein,
R¹ is unsubstituted alkyl, —COR¹¹, —CON(R¹¹)₂, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, or optionally substituted (C₁-C₄ alkylene)N(R¹²)—CON(R′)₂;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl.

One embodiment provides for a compound, or a pharmaceutically acceptable salt thereof, of Formula (IIa) wherein R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹).

One embodiment provides for a compound, or a pharmaceutically acceptable salt thereof, of Formula (IIa) wherein R¹ is unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R′)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂.

One embodiment provides for a compound, or a pharmaceutically acceptable salt thereof, of Formula (IIa) wherein R¹ is unsubstituted alkyl, —COR¹¹, —CON(R¹¹)₂, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, or optionally substituted (C₁-C₄ alkylene)N(R¹²)—CON(R′)₂.

One embodiment provides for a compound, or a pharmaceutically acceptable salt thereof, of Formula (IIa) wherein R¹ is —COR¹¹, —CON(R¹¹)₂, optionally substituted (C₁-C₄ alkylene)-N(R¹¹)₂, or optionally substituted (C₁-C₄ alkylene)N(R¹²)—COR¹¹.

One embodiment provides for a compound, or a pharmaceutically acceptable salt thereof, of Formula (IIa) wherein R¹ is —COR¹¹. One embodiment provides for a compound, or a pharmaceutically acceptable salt thereof, of Formula (IIa) wherein R¹ is —CON(R¹¹)₂. One embodiment provides for a compound, or a pharmaceutically acceptable salt thereof, of Formula (IIa) wherein R¹ is optionally substituted (C₁-C₄ alkylene)-N(R¹¹)₂. One embodiment provides for a compound, or a pharmaceutically acceptable salt thereof, of Formula (IIa) wherein R¹ is optionally substituted (C₁-C₄ alkylene)N(R¹²)—COR¹¹. One embodiment provides for a compound, or a pharmaceutically acceptable salt thereof, of Formula (IIa) wherein optionally substituted (C₁-C₄ alkylene) is —CH₂—.

One embodiment provides for a compound, or a pharmaceutically acceptable salt thereof, of Formula (IIa) wherein each R¹¹ is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl.

One embodiment provides for a compound, or a pharmaceutically acceptable salt thereof, of Formula (IIa) wherein R¹ is H. One embodiment provides for a compound, or a pharmaceutically acceptable salt thereof, of Formula (IIa) wherein R¹¹ is optionally substituted alkyl. One embodiment provides for a compound, or a pharmaceutically acceptable salt thereof, of Formula (IIa) wherein R¹¹ is hydroxy substituted alkyl. One embodiment provides for a compound, or a pharmaceutically acceptable salt thereof, of Formula (IIa) wherein R¹¹ is fluoro substituted alkyl. One embodiment provides for a compound, or a pharmaceutically acceptable salt thereof, of Formula (IIa) wherein R¹ is 2-fluoroethyl. One embodiment provides for a compound, or a pharmaceutically acceptable salt thereof, of Formula (IIa) wherein R¹¹ is fluoromethyl. One embodiment provides for a compound, or a pharmaceutically acceptable salt thereof, of Formula (IIa) wherein R¹¹ is difluoro substituted alkyl. One embodiment provides for a compound, or a pharmaceutically acceptable salt thereof, of Formula (IIa) wherein R¹¹ is 2,2-difluoroethyl. One embodiment provides for a compound, or a pharmaceutically acceptable salt thereof, of Formula (IIa) wherein R¹¹ is difluoromethyl. One embodiment provides for a compound, or a pharmaceutically acceptable salt thereof, of Formula (IIa) wherein R¹¹ is trifluoro substituted alkyl. One embodiment provides for a compound, or a pharmaceutically acceptable salt thereof, of Formula (IIa) wherein R¹¹ is 2,2,2-trifluoroethyl. One embodiment provides for a compound, or a pharmaceutically acceptable salt thereof, of Formula (IIa) wherein R¹¹ is trifluoromethyl. One embodiment provides for a compound, or a pharmaceutically acceptable salt thereof, of Formula (IIa) wherein R¹¹ is amino substituted alkyl. One embodiment provides for a compound, or a pharmaceutically acceptable salt thereof, of Formula (IIa) wherein R¹¹ is cyano substituted alkyl. One embodiment provides for a compound, or a pharmaceutically acceptable salt thereof, of Formula (IIa) wherein R¹¹ is optionally substituted alkenyl. One embodiment provides for a compound, or a pharmaceutically acceptable salt thereof, of Formula (IIa) wherein R¹¹ is optionally substituted carbocyclyl. One embodiment provides for a compound, or a pharmaceutically acceptable salt thereof, of Formula (IIa) wherein R¹¹ is optionally substituted carbocyclylalkyl. One embodiment provides for a compound, or a pharmaceutically acceptable salt thereof, of Formula (IIa) wherein R¹¹ is optionally substituted heterocyclyl. One embodiment provides for a compound, or a pharmaceutically acceptable salt thereof, of Formula (IIa) wherein R¹¹ is optionally substituted heterocyclylalkyl. One embodiment provides for a compound, or a pharmaceutically acceptable salt thereof, of Formula (IIa) wherein R¹¹ is optionally substituted heteroaryl. One embodiment provides for a compound, or a pharmaceutically acceptable salt thereof, of Formula (IIa) wherein R¹¹ is optionally substituted heteroarylalkyl.

One embodiment provides for a compound, or a pharmaceutically acceptable salt thereof, of Formula (IIa) wherein two R¹¹ groups together with the nitrogen to which they are attached join to form an optionally substituted N-heterocyclyl. One embodiment provides for a compound, or a pharmaceutically acceptable salt thereof, of Formula (IIa) wherein R¹¹ is vinyl, propan-2-yl, methyl, ethyl, cyclopropyl, cyclopentyl, azentidin-1-yl or allyl.

One embodiment provides for a compound, or a pharmaceutically acceptable salt thereof, of Formula (IIa) wherein Z is -L-G, optionally substituted (C₁-C₄ alkylene)-OCON(R¹³)₂, optionally substituted (C₁-C₄ alkylene)-N(R¹⁴)CON(R¹³)₂, optionally substituted (C₁-C₄ alkylene)-SO₂N(R¹³)₂, or optionally substituted (C₁-C₄ alkylene)-N(R¹⁴)SO₂R¹³. One embodiment provides for a compound, or a pharmaceutically acceptable salt thereof, of Formula (IIa) wherein Z is -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂.

One embodiment provides for a compound, or a pharmaceutically acceptable salt thereof, of Formula (IIa) wherein Z is -L-G.

One embodiment provides for a compound, or a pharmaceutically acceptable salt thereof, of Formula (IIa) wherein Z is morpholinomethyl, (8-oxa-3-azabicyclo[3.2.1]octan-3-yl)methyl, (2-oxa-5-azabicyclo[2.2.1]heptan-5-yl)methyl, (1,1-dioxidothiomorpholino)methyl, (oxetan-3-ylamino)methyl, ((methoxycyclobutyl)amino)methyl, (methylpiperazin-1-yl)methyl, (cyanopyrrolidin-1-yl)methyl, or (methoxypyrrolidin-1-yl)methyl.

One embodiment provides for a compound, or a pharmaceutically acceptable salt thereof, of Formula (IIa) wherein L is a bond or optionally substituted C₁-C₄ alkylene. One embodiment provides for a compound, or a pharmaceutically acceptable salt thereof, of Formula (IIa) wherein L is a bond. One embodiment provides for a compound, or a pharmaceutically acceptable salt thereof, of Formula (IIa) wherein L is an optionally substituted C₁-C₄ alkylene. One embodiment provides for a compound, or a pharmaceutically acceptable salt thereof, of Formula (IIa) wherein L is optionally substituted C₁. One embodiment provides for a compound, or a pharmaceutically acceptable salt thereof, of Formula (IIa) wherein L is —CH₂—.

One embodiment provides for a compound, or a pharmaceutically acceptable salt thereof, of Formula (IIa) wherein G is optionally substituted alkyl. One embodiment provides for a compound, or a pharmaceutically acceptable salt thereof, of Formula (IIa) wherein G is optionally substituted heterocyclyl. One embodiment provides for a compound, or a pharmaceutically acceptable salt thereof, of Formula (IIa) wherein G is optionally substituted morpholinyl, piperidinyl, piperazinyl, pyrolidinyl, imidazolyl, imidazolidinyl. One embodiment provides for a compound, or a pharmaceutically acceptable salt thereof, of Formula (IIa) wherein G is optionally substituted morpholinyl. One embodiment provides for a compound, or a pharmaceutically acceptable salt thereof, of Formula (IIa) wherein G is unsubstituted morpholinyl. One embodiment provides for a compound, or a pharmaceutically acceptable salt thereof, of Formula (IIa) wherein G is optionally substituted heteroaryl. One embodiment provides for a compound, or a pharmaceutically acceptable salt thereof, of Formula (IIa) wherein G is —N(R¹³)₂. One embodiment provides for a compound, or a pharmaceutically acceptable salt thereof, of Formula (IIa) wherein G is —OR¹³. One embodiment provides for a compound, or a pharmaceutically acceptable salt thereof, of Formula (IIa) wherein G is —CN.

One embodiment provides for a compound, or a pharmaceutically acceptable salt thereof, of Formula (IIa) wherein each R¹³ is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl.

One embodiment provides for a compound, or a pharmaceutically acceptable salt thereof, of Formula (IIa) wherein R¹³ is H. One embodiment provides for a compound, or a pharmaceutically acceptable salt thereof, of Formula (IIa) wherein R¹³ is optionally substituted alkyl. One embodiment provides for a compound, or a pharmaceutically acceptable salt thereof, of Formula (IIa) wherein R¹³ is optionally substituted carbocyclyl. One embodiment provides for a compound, or a pharmaceutically acceptable salt thereof, of Formula (IIa) wherein R¹³ is optionally substituted carbocyclylalkyl. One embodiment provides for a compound, or a pharmaceutically acceptable salt thereof, of Formula (IIa) wherein R¹³ is optionally substituted heterocyclyl. One embodiment provides for a compound, or a pharmaceutically acceptable salt thereof, of Formula (IIa) wherein R¹³ is optionally substituted heterocyclylalkyl. One embodiment provides for a compound, or a pharmaceutically acceptable salt thereof, of Formula (IIa) wherein R¹³ is optionally substituted heteroaryl. One embodiment provides for a compound, or a pharmaceutically acceptable salt thereof, of Formula (IIa) wherein R¹³ is optionally substituted heteroarylalkyl. One embodiment provides for a compound, or a pharmaceutically acceptable salt thereof, of Formula (IIa) wherein two R¹³ groups together with the nitrogen to which they are attached join to form an optionally substituted N-heterocyclyl.

One embodiment provides for a compound, or a pharmaceutically acceptable salt thereof, of Formula (IIa) wherein R¹ is not a hydroxy substituted alkyl.

One embodiment provides a compound, or a pharmaceutically acceptable salt thereof, having the structure of Formula (IIIa):

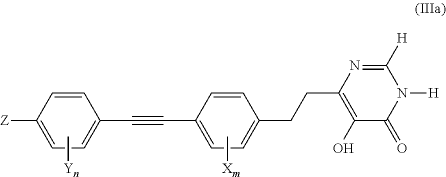

wherein,
n is 1 or 2 and m is 0, 1, or 2; or n is 0, 1, or 2 and m is 1 or 2;
X is halogen or optionally substituted C₁-C₃ alkyl;
Y is halogen or optionally substituted C₁-C₃ alkyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl

One embodiment provides for a compound, or a pharmaceutically acceptable salt thereof, of Formula (IIIa):
wherein,
n is 1 or 2 and m is 0, 1, or 2; or n is 0, 1, or 2 and m is 1 or 2;
X is halogen or optionally substituted C₁-C₃ alkyl;
Y is halogen or optionally substituted C₁-C₃ alkyl;
Z is H, -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂, —SO₂(R¹³);
L is a bond, optionally substituted C₁-C₄ alkylene, —C(O)—, or —SO₂—;
G is optionally substituted alkyl, optionally substituted heterocyclyl, optionally substituted heteroaryl, —N(R¹³)₂, —OR¹³, or —CN;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl.

One embodiment provides for a compound, or a pharmaceutically acceptable salt thereof, of Formula (IIIa) wherein n is 0 or 1. One embodiment provides for a compound, or a pharmaceutically acceptable salt thereof, of Formula (IIIa) wherein m is 0 or 1. One embodiment provides for a compound, or a pharmaceutically acceptable salt thereof, of Formula (IIIa) wherein n is 1 or 2. One embodiment provides for a compound, or a pharmaceutically acceptable salt thereof, of Formula (IIIa) wherein m is 1 or 2. One embodiment provides for a compound, or a pharmaceutically acceptable salt thereof, of Formula (IIIa) wherein X is halogen. One embodiment provides for a compound, or a pharmaceutically acceptable salt thereof, of Formula (IIIa) wherein Y is halogen.

One embodiment provides for a compound, or a pharmaceutically acceptable salt thereof, of Formula (IIIa) wherein Z is -L-G, optionally substituted (C1-C4 alkylene)-OCON(R13)2, optionally substituted (C1-C4 alkylene)-N(R14)CON(R13)2, optionally substituted (C1-C4 alkylene)-SO2N(R13)2, or optionally substituted (C1-C4 alkylene)-N(R14)SO2R13. One embodiment provides for a compound, or a pharmaceutically acceptable salt thereof, of Formula (IIIa) wherein Z is -L-G, optionally substituted (C1-C4 alkylene)-OCON(R13)2, optionally substituted (C1-C4 alkylene)-N(R14)CON(R13)2, or optionally substituted (C1-C4 alkylene)-SO2N(R13)2.

One embodiment provides for a compound, or a pharmaceutically acceptable salt thereof, of Formula (IIIa) wherein Z is -L-G.

One embodiment provides for a compound, or a pharmaceutically acceptable salt thereof, of Formula (IIIa) wherein Z is morpholinomethyl, (8-oxa-3-azabicyclo[3.2.1]octan-3-yl)methyl, (2-oxa-5-azabicyclo[2.2.1]heptan-5-yl)methyl, (1,1-dioxidothiomorpholino)methyl, (oxetan-3-ylamino)methyl, ((methoxycyclobutyl)amino)methyl, (methylpiperazin-1-yl)methyl, (cyanopyrrolidin-1-yl)methyl, or (methoxypyrrolidin-1-yl)methyl.

One embodiment provides for a compound, or a pharmaceutically acceptable salt thereof, of Formula (IIIa) wherein L is a bond or optionally substituted C1-C4 alkylene. One embodiment provides for a compound, or a pharmaceutically acceptable salt thereof, of Formula (IIIa) wherein L is a bond. One embodiment provides for a compound, or a pharmaceutically acceptable salt thereof, of Formula (IIIa) wherein L is an optionally substituted C1-C4 alkylene. One embodiment provides for a compound, or a pharmaceutically acceptable salt thereof, of Formula (IIIa) wherein L is optionally substituted C1. One embodiment provides for a compound, or a pharmaceutically acceptable salt thereof, of Formula (IIIa) wherein L is —CH2-. One embodiment provides for a compound, or a pharmaceutically acceptable salt thereof, of Formula (IIIa) wherein G is optionally substituted alkyl. One embodiment provides for a compound, or a pharmaceutically acceptable salt thereof, of Formula (IIIa) wherein G is optionally substituted heterocyclyl.

One embodiment provides for a compound, or a pharmaceutically acceptable salt thereof, of Formula (IIIa) wherein G is optionally substituted morpholinyl, piperidinyl, piperazinyl, pyrolidinyl, imidazolyl, imidazolidinyl. One embodiment provides for a compound, or a pharmaceutically acceptable salt thereof, of Formula (IIIa) wherein G is optionally substituted morpholinyl. One embodiment provides for a compound, or a pharmaceutically acceptable salt thereof, of Formula (IIIa) wherein G is unsubstituted morpholinyl. One embodiment provides for a compound, or a pharmaceutically acceptable salt thereof, of Formula (IIIa) wherein G is optionally substituted heteroaryl. One embodiment provides for a compound, or a pharmaceutically acceptable salt thereof, of Formula (IIIa) wherein G is —N(R13)2. One embodiment provides for a compound, or a pharmaceutically acceptable salt thereof, of Formula (IIIa) wherein G is —OR13. One embodiment provides for a compound, or a pharmaceutically acceptable salt thereof, of Formula (IIIa) wherein G is —CN.

One embodiment provides for a compound, or a pharmaceutically acceptable salt thereof, of Formula (IIIa) wherein each R13 is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R13 groups together with the nitrogen to which they are attached join to form an optionally substituted N-heterocyclyl.

One embodiment provides for a compound, or a pharmaceutically acceptable salt thereof, of Formula (IIIa) wherein R13 is H. One embodiment provides for a compound, or a pharmaceutically acceptable salt thereof, of Formula (IIIa) wherein R13 is optionally substituted alkyl. One embodiment provides for a compound, or a pharmaceutically acceptable salt thereof, of Formula (IIIa) wherein R13 is optionally substituted carbocyclyl. One embodiment provides for a compound, or a pharmaceutically acceptable salt thereof, of Formula (IIIa) wherein R13 is optionally substituted carbocyclylalkyl. One embodiment provides for a compound, or a pharmaceutically acceptable salt thereof, of Formula (IIIa) wherein R13 is optionally substituted heterocyclyl. One embodiment provides for a compound, or a pharmaceutically acceptable salt thereof, of Formula (IIIa) wherein R13 is optionally substituted heterocyclylalkyl. One embodiment provides for a compound, or a pharmaceutically acceptable salt thereof, of Formula (IIIa) wherein R13 is optionally substituted heteroaryl. One embodiment provides for a compound, or a pharmaceutically acceptable salt thereof, of Formula (IIIa) wherein R13 is optionally substituted heteroarylalkyl. One embodiment provides for a compound, or a pharmaceutically acceptable salt thereof, of Formula (IIIa) wherein two R13 groups together with the nitrogen to which they are attached join to form an optionally substituted N-heterocyclyl.

In some embodiments, the heterocyclic LpxC inhibitory compound described in Formula (I), (Ia), (IIa) or (IIIa) has a structure provided in Table 1.

TABLE 1

| Compound | | | Mass |
|---|---|---|---|
| No. | Name | Structure | [M + H] |
| 1 | N-(2,2-difluoroethyl)- 2-(5-hydroxy-6-oxo- 1,6-dihydropyrimidin- 4-yl)-3-(4-((4- (morpholinomethyl) phenyl)ethynyl)phenyl) propanamide | 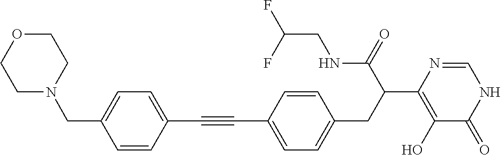 [SMILES: O=C(NCC(F)F)C(Cc1ccc(C#Cc2ccc(CN3CCOCC3)cc2)cc1)c1nc[nH]c(=O)c1O] | 523.21 |
| 2 | 2-(5-hydroxy-6-oxo- 1,6-dihydropyrimidin- 4-yl)-3-(4-((4- (morpholinomethyl) phenyl)ethynyl)phenyl)- N-(2,2,2-trifluoroethyl) propanamide | 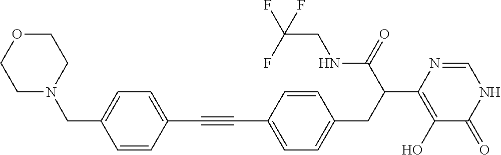 [SMILES: O=C(NCC(F)(F)F)C(Cc1ccc(C#Cc2ccc(CN3CCOCC3)cc2)cc1)c1nc[nH]c(=O)c1O] | 541.20 |
| 3 | N-(2-fluoroethyl)-2- (5-hydroxy-6-oxo-1,6- dihydropyrimidin-4- yl)-3-(4-((4- (morpholinomethyl) phenyl)ethynyl)phenyl) propanamide | 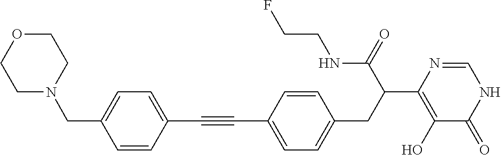 [SMILES: O=C(NCCF)C(Cc1ccc(C#Cc2ccc(CN3CCOCC3)cc2)cc1)c1nc[nH]c(=O)c1O] | 505.22 |
| 4 | 2-(5-hydroxy-6-oxo- 1,6-dihydropyrimidin- 4-yl)-N-(isoxazol-4- yl)-3-(4-((4- (morpholinomethyl) phenyl)ethynyl) phenyl) propanamide | 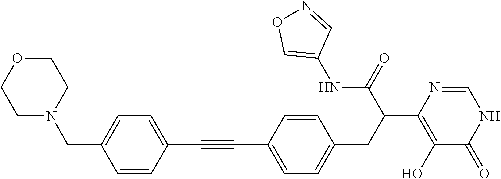 [SMILES: O=C(Nc1cnoc1)C(Cc1ccc(C#Cc2ccc(CN3CCOCC3)cc2)cc1)c1nc[nH]c(=O)c1O] | 526.20 |
| 5 | 2-(5-hydroxy-6-oxo- 1,6-dihydropyrimidin- 4-yl)-N-methyl-3-(4- ((4- (morpholinomethyl) phenyl)ethynyl)phenyl) propanamide | 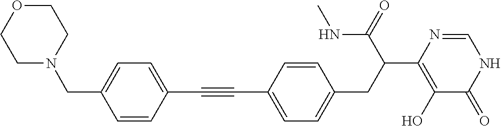 [SMILES: CNC(=O)C(Cc1ccc(C#Cc2ccc(CN3CCOCC3)cc2)cc1)c1nc[nH]c(=O)c1O] | 473.21 |
| 6 | N-allyl-2-(5-hydroxy- 6-oxo-1,6- dihydropyrimidin- 4-yl)-3-(4-((4- (morpholinomethyl) phenyl)ethynyl) phenyl)propanamide | 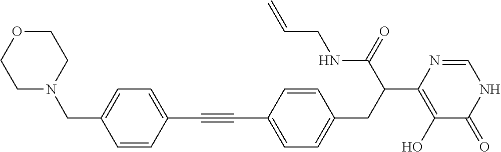 [SMILES: C=CCNC(=O)C(Cc1ccc(C#Cc2ccc(CN3CCOCC3)cc2)cc1)c1nc[nH]c(=O)c1O] | 499.23 |
| 7 | 2-(5-hydroxy-6-oxo- 1,6-dihydropyrimidin- 4-yl)-N-(2- hydroxyethyl)-3-(4- ((4-(morpholinomethyl) phenyl)ethynyl)phenyl) propanamide | 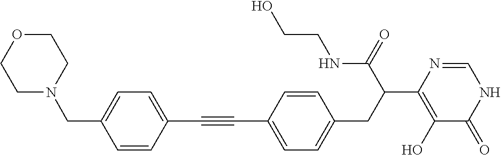 [SMILES: O=C(NCCO)C(Cc1ccc(C#Cc2ccc(CN3CCOCC3)cc2)cc1)c1nc[nH]c(=O)c1O] | 503.35 |
| 8 | 2-(5-hydroxy-6-oxo- 1,6-dihydropyrimidin- 4-yl)-3-(4-((4- (morpholinomethyl) phenyl)ethynyl)phenyl)- N-(1H-pyrazol-5- yl)propanamide | 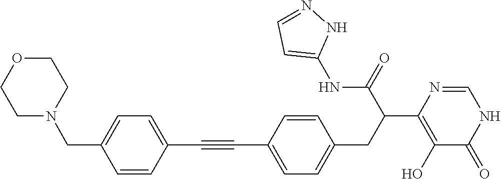 [SMILES: O=C(Nc1ccn[nH]1)C(Cc1ccc(C#Cc2ccc(CN3CCOCC3)cc2)cc1)c1nc[nH]c(=O)c1O] | 525.35 |
| 9 | 2-(5-hydroxy-6-oxo- 1,6-dihydropyrimidin- 4-yl)-N-(1H-imidazol- 5-yl)-3-(4-((4- (morpholinomethyl) phenyl)ethynyl)phenyl) propanamide | 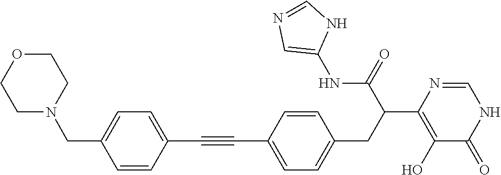 [SMILES: O=C(Nc1cnc[nH]1)C(Cc1ccc(C#Cc2ccc(CN3CCOCC3)cc2)cc1)c1nc[nH]c(=O)c1O] | 525.28 |
| 10 | N-(2-aminoethyl)-2- (5-hydroxy-6-oxo-1,6- dihydropyrimidin-4- yl)-3-(4-((4- (morpholinomethyl) phenyl)ethynyl)phenyl) propanamide | 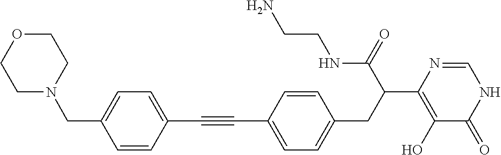 [SMILES: NCCNC(=O)C(Cc1ccc(C#Cc2ccc(CN3CCOCC3)cc2)cc1)c1nc[nH]c(=O)c1O] | 502.28 |
| 11 | 2-(5-hydroxy-6- oxo-1,6- dihydropyrimidin- 4-yl)-3-(4-((4- (morpholinomethyl) phenyl)ethynyl)phenyl)- N-(1H-tetrazol-5- yl)propanamide | 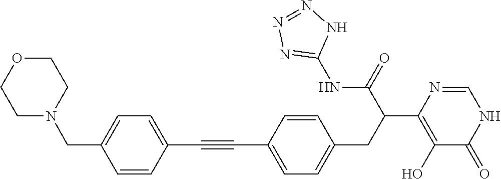 [SMILES: O=C(Nc1nnn[nH]1)C(Cc1ccc(C#Cc2ccc(CN3CCOCC3)cc2)cc1)c1nc[nH]c(=O)c1O] | 527.23 |
| 12 | 2-fluoro-N-(2-(5- hydroxy-6-oxo-1,6- dihydropyrimidin- 4-yl)-3-(4-((4- (morpholinomethyl) phenyl)ethynyl) phenyl)propyl) acetamide | 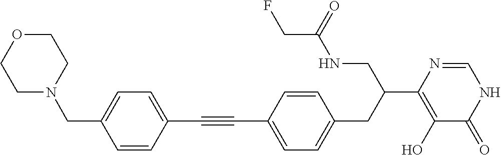 [SMILES: O=C(CF)NCC(Cc1ccc(C#Cc2ccc(CN3CCOCC3)cc2)cc1)c1nc[nH]c(=O)c1O] | 505.23 |
| 13 | N-(3-(4-((4-((8-oxa-3- azabicyclo[3.2.1]octan- 3-yl)methyl)phenyl) ethynyl)phenyl)-2-(5- hydroxy-6-oxo-1,6- dihydropyrimidin- 4-yl)propyl)-2- fluoroacetamide | 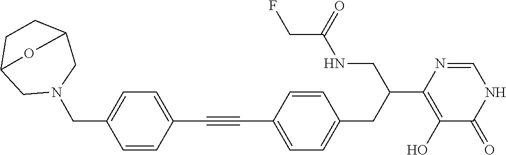 [SMILES: O=C(CF)NCC(Cc1ccc(C#Cc2ccc(CN3CC4CCC(C3)O4)cc2)cc1)c1nc[nH]c(=O)c1O] | 531.34 |
| 14 | (R)-N-(2-(5-hydroxy- 6-oxo-1,6- dihydropyrimidin-4- yl)-3-(4-((4- (morpholinomethyl) phenyl)ethynyl)phenyl) propyl)acetamide | 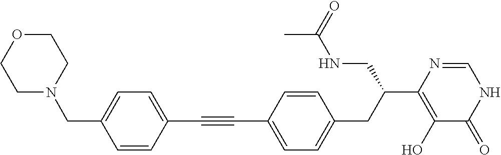 [SMILES: CC(=O)NC[C@@H](Cc1ccc(C#Cc2ccc(CN3CCOCC3)cc2)cc1)c1nc[nH]c(=O)c1O] | 587.32 |
| 15 | 2,2-difluoro-N-(2-(5- hydroxy-6-oxo-1,6- dihydropyrimidin-4- yl)-3-(4-((4- (morpholinomethyl) phenyl)ethynyl)phenyl) propyl)acetamide | 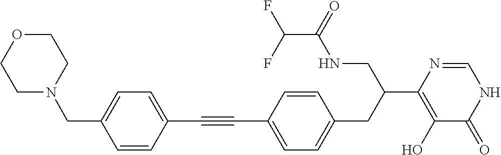 [SMILES: O=C(NCC(Cc1ccc(C#Cc2ccc(CN3CCOCC3)cc2)cc1)c1nc[nH]c(=O)c1O)C(F)F] | 523.12 |
| 16 | 2-fluoro-N-(2-(5- hydroxy-6-oxo-1,6- dihydropyrimidin- 4-yl)-3-(4-((4-(2- morpholinoethyl) phenyl)ethynyl)phenyl) propyl)acetamide | 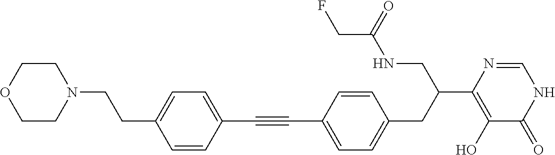 [SMILES: O=C(CF)NCC(Cc1ccc(C#Cc2ccc(CCN3CCOCC3)cc2)cc1)c1nc[nH]c(=O)c1O] | 519.34 |
| 17 | 2-fluoro-N-(2-(5- hydroxy-6-oxo-1,6- dihydropyrimidin- 4-yl)-3-(4-((4-(2- morpholinoethyl) phenyl)ethynyl) phenyl)propyl) acetamide | 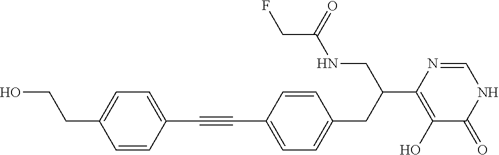 [SMILES: O=C(CF)NCC(Cc1ccc(C#Cc2ccc(CCO)cc2)cc1)c1nc[nH]c(=O)c1O] | 519.21 |
| 18 | 2,2,2-trifluoro-N-(2- (5-hydroxy-6-oxo-1,6- dihydropyrimidin-4- yl)-3-(4-((4- (morpholinomethyl) phenyl)ethynyl)phenyl) propyl)acetamide | 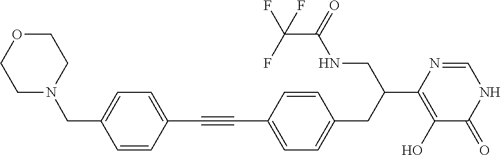 [SMILES: O=C(NCC(Cc1ccc(C#Cc2ccc(CN3CCOCC3)cc2)cc1)c1nc[nH]c(=O)c1O)C(F)(F)F] | 541.13 |
| 19 | N-(2-(5-hydroxy-6- oxo-1,6- dihydropyrimidin-4- yl)-3-(4-((4- (morpholinomethyl) phenyl)ethynyl)phenyl) propyl)acetamide | 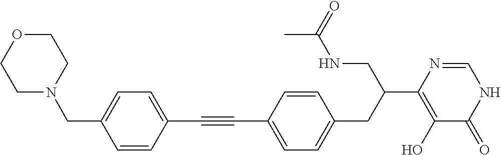 [SMILES: CC(=O)NCC(Cc1ccc(C#Cc2ccc(CN3CCOCC3)cc2)cc1)c1nc[nH]c(=O)c1O] | 587.37 |
| 20 | N-(2-(5-hydroxy-6- oxo-1,6- dihydropyrimidin-4- yl)-3-(4-((4- (morpholinomethyl) phenyl)ethynyl)phenyl) propyl)acrylamide | 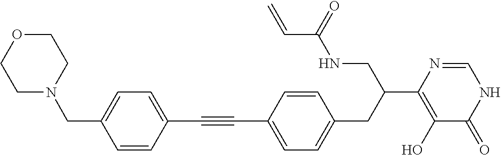 [SMILES: C=CC(=O)NCC(Cc1ccc(C#Cc2ccc(CN3CCOCC3)cc2)cc1)c1nc[nH]c(=O)c1O] | 599.32 |
| 21 | methyl (3-(4-((4-((8- oxa-3- azabicyclo[3.2.1]octan- 3-yl)methyl)phenyl) ethynyl)phenyl)-2-(5- hydroxy-6-oxo-1,6- dihydropyrimidin- 4-yl)propyl)carbamate | 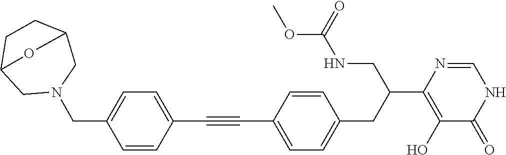 [SMILES: COC(=O)NCC(Cc1ccc(C#Cc2ccc(CN3CC4CCC(C3)O4)cc2)cc1)c1nc[nH]c(=O)c1O] | 529.12 |
| 22 | N-(3-(3-fluoro-4-((4- (morpholinomethyl) phenyl)ethynyl) phenyl)-2-(5- hydroxy-6-oxo-1,6- dihydropyrimidin-4- yl)propyl)acetamide | 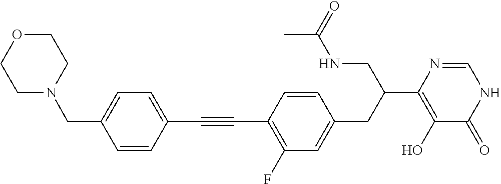 [SMILES: CC(=O)NCC(Cc1ccc(C#Cc2ccc(CN3CCOCC3)cc2)c(F)c1)c1nc[nH]c(=O)c1O] | 505.32 |
| 23 | N-(3-(4-((4-(((1R,4R)- 2-oxa-5- azabicyclo[2.2.1]heptan- 5-yl)methyl)phenyl) ethynyl)phenyl)-2-(5- hydroxy-6-oxo-1,6- dihydropyrimidin- 4-yl)propyl)-2- fluoroacetamide | 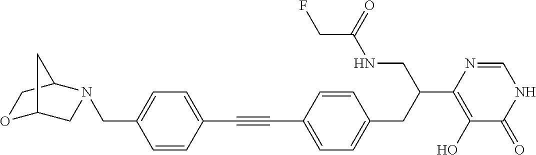 [SMILES: O=C(CF)NCC(Cc1ccc(C#Cc2ccc(CN3CC4CC3CO4)cc2)cc1)c1nc[nH]c(=O)c1O] | 517.22 |
| 24 | N-(3-(3-chloro-4-((4- (morpholinomethyl) phenyl)ethynyl)phenyl)- 2-(5-hydroxy-6-oxo-1,6- dihydropyrimidin- 4-yl)propyl)acetamide | 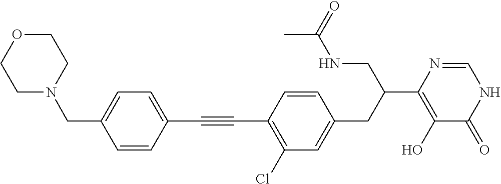 [SMILES: CC(=O)NCC(Cc1ccc(C#Cc2ccc(CN3CCOCC3)cc2)c(Cl)c1)c1nc[nH]c(=O)c1O] | 522.15 |
| 25 | N-(2-(5-hydroxy-6-oxo- 1,6-dihydropyrimidin-4- yl)-3-(4-((4- (morpholinomethyl) phenyl)ethynyl)phenyl) propyl) cyclopropanecarboxamide | 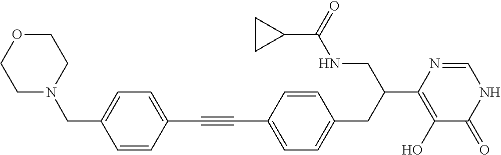 [SMILES: O=C(NCC(Cc1ccc(C#Cc2ccc(CN3CCOCC3)cc2)cc1)c1nc[nH]c(=O)c1O)C1CC1] | 513.21 |
| 26 | N-(2-(5-hydroxy-6-oxo- 1,6-dihydropyrimidin-4- yl)-3-(4-((4- (morpholinomethyl) phenyl)ethynyl)phenyl) propyl)propionamide | 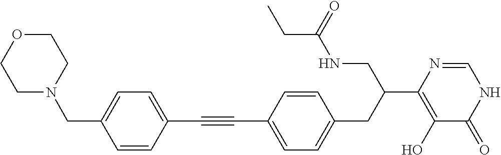 [SMILES: CCC(=O)NCC(Cc1ccc(C#Cc2ccc(CN3CCOCC3)cc2)cc1)c1nc[nH]c(=O)c1O] | 501.20 |
| 27 | N-(3-(4-((4-((1,1- dioxidothiomorpholino) methyl)phenyl)ethynyl) phenyl)-2-(5-hydroxy-6- oxo-1,6- dihydropyrimidin- 4-yl)propyl)acetamide | 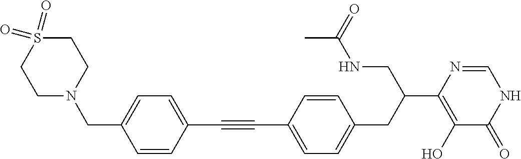 [SMILES: CC(=O)NCC(Cc1ccc(C#Cc2ccc(CN3CCS(=O)(=O)CC3)cc2)cc1)c1nc[nH]c(=O)c1O] | 535.10 |
| 28 | N-(3-(4-((4-(((1R,4R)-2- oxa-5- azabicyclo[2.2.1]heptan- 5-yl)methyl)phenyl) ethynyl)phenyl)-2-(5- hydroxy-6-oxo-1,6- dihydropyrimidin- 4-yl)propyl)acetamide | 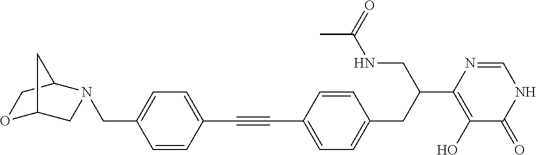 [SMILES: CC(=O)NCC(Cc1ccc(C#Cc2ccc(CN3CC4CC3CO4)cc2)cc1)c1nc[nH]c(=O)c1O] | 499.13 |
| 29 | N-(2-(5-hydroxy-6-oxo- 1,6-dihydropyrimidin-4- yl)-3-(4-((4-((oxetan-3- ylamino)methyl)phenyl) ethynyl)phenyl)propyl) acetamide | 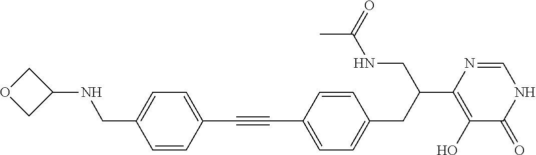 [SMILES: CC(=O)NCC(Cc1ccc(C#Cc2ccc(CNC3COC3)cc2)cc1)c1nc[nH]c(=O)c1O] | 473.21 |
| 30 | 2-fluoro-N-(2-(5- hydroxy-6-oxo-1,6- dihydropyrimidin-4- yl)-3-(4-((4-(((3- methoxycyclobutyl) amino)methyl)phenyl) ethynyl)phenyl)propyl) acetamide | 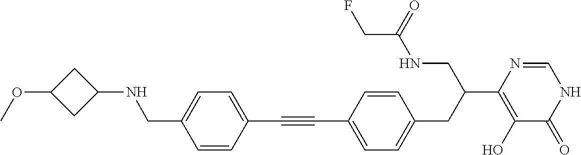 [SMILES: COC1CC(NCc2ccc(C#Cc3ccc(CC(CNC(=O)CF)c4nc[nH]c(=O)c4O)cc3)cc2)C1] | 519.34 |
| 31 | 4-((4-(3-acetamido-2- (5-hydroxy-6-oxo-1,6- dihydropyrimidin-4- yl)propyl)phenyl) ethynyl)benzyl carbamate | 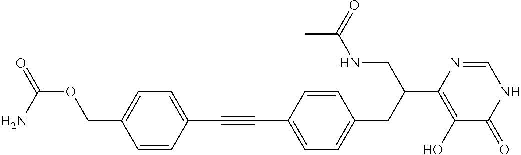 [SMILES: CC(=O)NCC(Cc1ccc(C#Cc2ccc(COC(N)=O)cc2)cc1)c1nc[nH]c(=O)c1O] | 461.18 |
| 32 | N-(2-(5-hydroxy-6- oxo-1,6- dihydropyrimidin-4- yl)-3-(4-((4-((4- methylpiperazin-1- yl)methyl)phenyl) ethynyl)phenyl)propyl) acetamide | 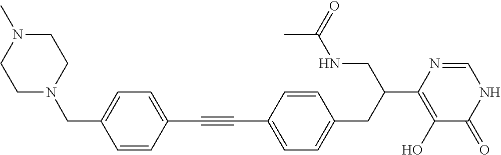 [SMILES: CC(=O)NCC(Cc1ccc(C#Cc2ccc(CN3CCN(C)CC3)cc2)cc1)c1nc[nH]c(=O)c1O] | 500.25 |
| 33 | 3-hydroxy-N-(2-(5- hydroxy-6-oxo-1,6- dihydropyrimidin- 4-yl)-3-(4-((4- (morpholinomethyl) phenyl)ethynyl)phenyl) propyl)propanamide | 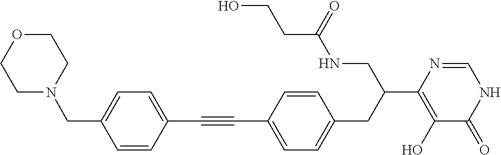 [SMILES: O=C(CCO)NCC(Cc1ccc(C#Cc2ccc(CN3CCOCC3)cc2)cc1)c1nc[nH]c(=O)c1O] | 517.24 |
| 34 | N-(3-(4-((4- (cyanomethyl)phenyl) ethynyl)phenyl)-2-(5- hydroxy-6-oxo-1,6- dihydropyrimidin-4- yl)propyl)acetamide | 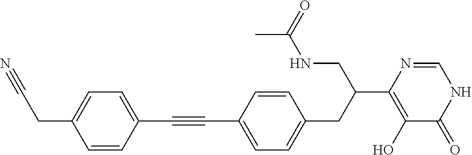 [SMILES: CC(=O)NCC(Cc1ccc(C#Cc2ccc(CC#N)cc2)cc1)c1nc[nH]c(=O)c1O] | 427.12 |
| 35 | N-(3-(4-((4-((1H- tetrazol-1-yl)methyl) phenyl)ethynyl) phenyl)-2-(5- hydroxy-6-oxo- 1,6-dihydropyrimidin- 4-yl)propyl)acetamide | 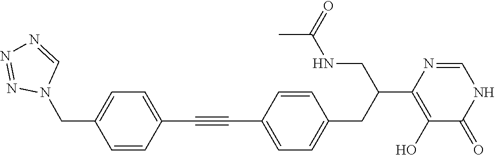 [SMILES: CC(=O)NCC(Cc1ccc(C#Cc2ccc(Cn3cnnn3)cc2)cc1)c1nc[nH]c(=O)c1O] | 470.18 |
| 36 | 1-hydroxy-N-(2-(5- hydroxy-6-oxo-1,6- dihydropyrimidin- 4-yl)-3-(4-((4- (morpholinomethyl) phenyl)ethynyl)phenyl) propyl)cyclopropane-1- carboxamide | 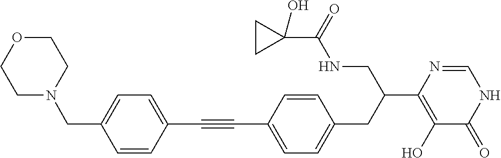 [SMILES: O=C(NCC(Cc1ccc(C#Cc2ccc(CN3CCOCC3)cc2)cc1)c1nc[nH]c(=O)c1O)C1(O)CC1] | 529.23 |
| 37 | 2-hydroxy-N-(2-(5- hydroxy-6-oxo-1,6- dihydropyrimidin- 4-yl)-3-(4-((4- (morpholinomethyl) phenyl)ethynyl)phenyl) propyl)acetamide | 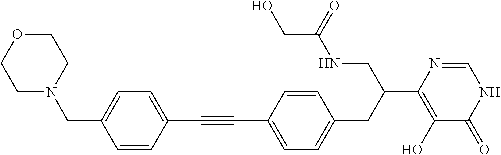 [SMILES: O=C(CO)NCC(Cc1ccc(C#Cc2ccc(CN3CCOCC3)cc2)cc1)c1nc[nH]c(=O)c1O] | 503.11 |
| 38 | 2-fluoro-N-(2-(5- hydroxy-6-oxo-1,6- dihydropyrimidin-4- yl)-3-(4-((4-((3- hydroxyazetidin-1- yl)methyl)phenyl) ethynyl)phenyl) propyl)acetamide | 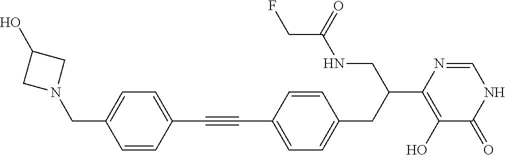 [SMILES: O=C(CF)NCC(Cc1ccc(C#Cc2ccc(CN3CC(O)C3)cc2)cc1)c1nc[nH]c(=O)c1O] | 491.20 |
| 39 | methyl (3-(4-((4-(((1- chloro-3- hydroxypropan-2- yl)amino)methyl) phenyl)ethynyl)phenyl)- 2-(5-hydroxy-6-oxo- 1,6-dihydropyrimidin- 4-yl)propyl)carbamate | 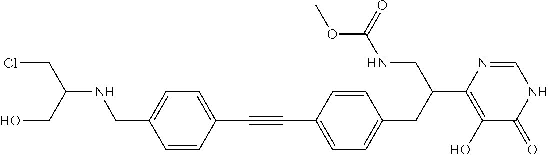 [SMILES: COC(=O)NCC(Cc1ccc(C#Cc2ccc(CNC(CO)CCl)cc2)cc1)c1nc[nH]c(=O)c1O] | 526.18 |
| 40 | 2-fluoro-N-(2-(5- hydroxy-6-oxo-1,6- dihydropyrimidin- 4-yl)-3-(4-((4-(((3- hydroxycyclobutyl) amino)methyl)phenyl) ethynyl)phenyl)propyl) acetamide | 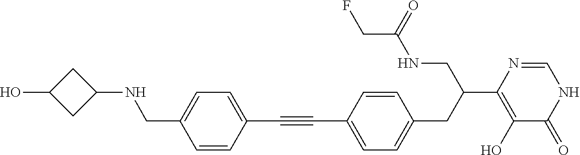 [SMILES: O=C(CF)NCC(Cc1ccc(C#Cc2ccc(CNC3CC(O)C3)cc2)cc1)c1nc[nH]c(=O)c1O] | 505.22 |
| 41 | N-(2-(5-hydroxy-6-oxo- 1,6-dihydropyrimidin- 4-yl)-3-(4-((4-(((3- methoxycyclobutyl) amino)methyl)phenyl) ethynyl)phenyl)propyl) acetamide | 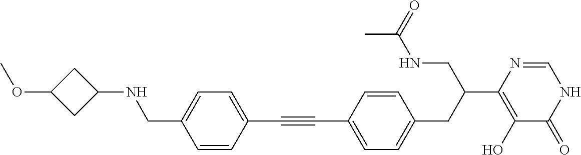 [SMILES: COC1CC(NCc2ccc(C#Cc3ccc(CC(CNC(C)=O)c4nc[nH]c(=O)c4O)cc3)cc2)C1] | 501.25 |
| 42 | 2-fluoro-N-(3-(5- hydroxy-6-oxo-1,6- dihydropyrimidin-4- yl)-4-(4-((4- (morpholinomethyl) phenyl)ethynyl)phenyl) butan-2-yl)acetamide | 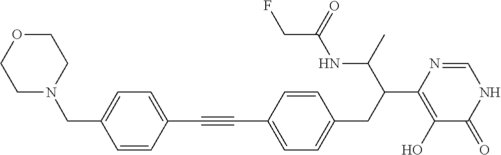 [SMILES: CC(NC(=O)CF)C(Cc1ccc(C#Cc2ccc(CN3CCOCC3)cc2)cc1)c1nc[nH]c(=O)c1O] | 519.45 |
| 43 | N-(2-(5-hydroxy-6- oxo-1,6- dihydropyrimidin-4- yl)-3-(4-((4- (morpholinomethyl) phenyl)ethynyl)phenyl) propyl)-P,P- dimethylphosphinic amide | 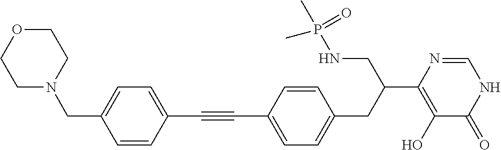 [SMILES: CP(C)(=O)NCC(Cc1ccc(C#Cc2ccc(CN3CCOCC3)cc2)cc1)c1nc[nH]c(=O)c1O] | 521.21 |
| 44 | 2-fluoro-N-(2-(5- hydroxy-6-oxo-1,6- dihydropyrimidin- 4-yl)-3-(4-((4- (pyrrolidin-1- ylmethyl)phenyl) ethynyl)phenyl) propyl)acetamide | 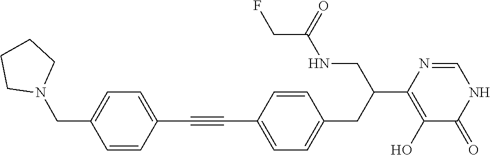 [SMILES: O=C(CF)NCC(Cc1ccc(C#Cc2ccc(CN3CCCC3)cc2)cc1)c1nc[nH]c(=O)c1O] | 489.23 |
| 45 | N-(3-(4-((4-(((R)-3- aminopyrrolidin-1- yl)methyl)phenyl) ethynyl)phenyl)-2-(5- hydroxy-6-oxo-1,6- dihydropyrimidin- 4-yl)propyl)-2- fluoroacetamide | 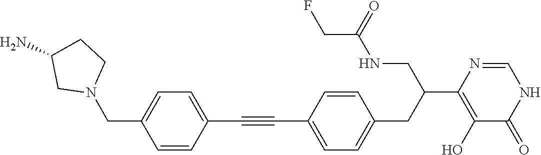 [SMILES: N[C@@H]1CCN(Cc2ccc(C#Cc3ccc(CC(CNC(=O)CF)c4nc[nH]c(=O)c4O)cc3)cc2)C1] | 504.24 |
| 46 | 2-(azetidin-1-yl)-N-(2- (5-hydroxy-6-oxo-1,6- dihydropyrimidin-4-yl)- 3-(4-((4- (morpholinomethyl) phenyl)ethynyl)phenyl) propyl)acetamide | 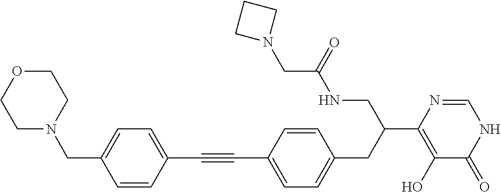 [SMILES: O=C(CN1CCC1)NCC(Cc1ccc(C#Cc2ccc(CN3CCOCC3)cc2)cc1)c1nc[nH]c(=O)c1O] | 542.27 |
| 47 | 3-amino-N-(2-(5- hydroxy-6-oxo-1,6- dihydropyrimidin- 4-yl)-3-(4-((4- (morpholinomethyl) phenyl)ethynyl)phenyl) propyl)propanamide | 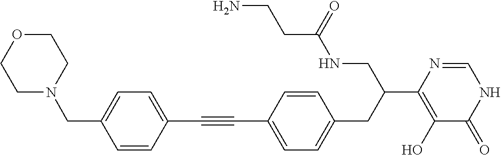 [SMILES: NCCC(=O)NCC(Cc1ccc(C#Cc2ccc(CN3CCOCC3)cc2)cc1)c1nc[nH]c(=O)c1O] | 516.26 |
| 48 | N-(2-(5-hydroxy-6-oxo- 1,6-dihydropyrimidin-4- yl)-3-(4-((4- (morpholinomethyl) phenyl)ethynyl)phenyl) propyl)-1H-pyrazole-4- carboxamide | 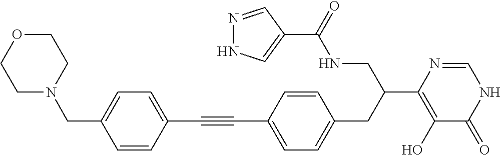 [SMILES: O=C(NCC(Cc1ccc(C#Cc2ccc(CN3CCOCC3)cc2)cc1)c1nc[nH]c(=O)c1O)c1cn[nH]c1] | 539.24 |
| 49 | methyl (2-(5-hydroxy- 6-oxo-1,6- dihydropyrimidin- 4-yl)-3-(4-((4-((3- hydroxyazetidin-1- yl)methyl)phenyl) ethynyl)phenyl)propyl) carbamate | 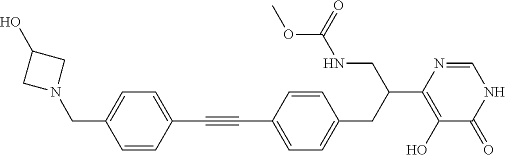 [SMILES: COC(=O)NCC(Cc1ccc(C#Cc2ccc(CN3CC(O)C3)cc2)cc1)c1nc[nH]c(=O)c1O] | 489.20 |
| 50 | N-(2-(5-hydroxy-6-oxo- 1,6-dihydropyrimidin-4- yl)-3-(4-((4- (morpholinomethyl) phenyl)ethynyl)phenyl) propyl)-N- methylpropionamide | 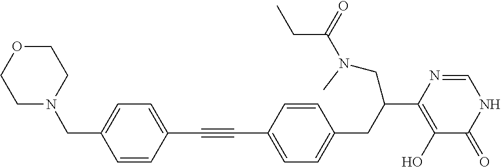 [SMILES: CCC(=O)N(C)CC(Cc1ccc(C#Cc2ccc(CN3CCOCC3)cc2)cc1)c1nc[nH]c(=O)c1O] | 5155.21 |
| 51 | 1-(2-(5-hydroxy-6-oxo- 1,6-dihydropyrimidin-4- yl)-3-(4-((4- (morpholinomethyl) phenyl)ethynyl)phenyl) propyl)urea | 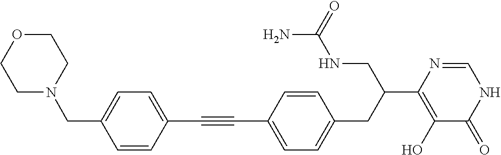 [SMILES: NC(=O)NCC(Cc1ccc(C#Cc2ccc(CN3CCOCC3)cc2)cc1)c1nc[nH]c(=O)c1O] | 488.23 |
| 52 | 1-(4-((4-(3-acetamido-2- (5-hydroxy-6-oxo-1,6- dihydropyrimidin-4-yl) propyl)phenyl)ethynyl) benzyl)pyrrolidine-3- carboxylic acid | 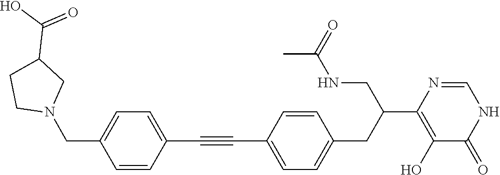 [SMILES: CC(=O)NCC(Cc1ccc(C#Cc2ccc(CN3CCC(C(=O)O)C3)cc2)cc1)c1nc[nH]c(=O)c1O] | 515.22 |
| 53 | N-(3-(4-((4-((1H- tetrazol-5-yl)methyl) phenyl)ethynyl) phenyl)-2-(5-hydroxy- 6-oxo-1,6- dihydropyrimidin-4- yl)propyl)acetamide | 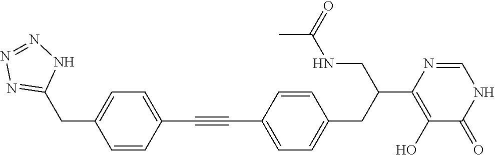 [SMILES: CC(=O)NCC(Cc1ccc(C#Cc2ccc(Cc3nnn[nH]3)cc2)cc1)c1nc[nH]c(=O)c1O] | 470.10 |
| 54 | 2-amino-N-(2-(5- hydroxy-6-oxo-1,6- dihydropyrimidin-4- yl)-3-(4-((4- (morpholinomethyl) phenyl)ethynyl)phenyl) propyl)acetamide | 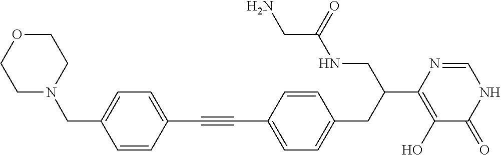 [SMILES: NCC(=O)NCC(Cc1ccc(C#Cc2ccc(CN3CCOCC3)cc2)cc1)c1nc[nH]c(=O)c1O] | 502.24 |
| 55 | 6-(1-((2-fluoroethyl) amino)-3-(4-((4-((2- oxopyrrolidin-1- yl)methyl)phenyl) ethynyl)phenyl) propan-2-yl)-5- hydroxypyrimidin- 4(3H)-one | 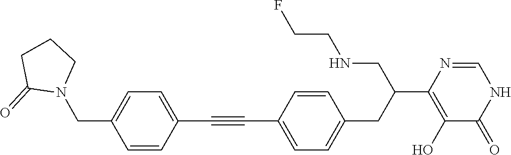 [SMILES: O=C1CCCN1Cc1ccc(C#Cc2ccc(CC(CNCCF)c3nc[nH]c(=O)c3O)cc2)cc1] | 489.23 |
| 56 | 6-(1-(allylamino)-3-(4- ((4-(morpholinomethyl) phenyl)ethynyl)phenyl) propan-2-yl)-5- hydroxypyrimidin- 4(3H)-one | 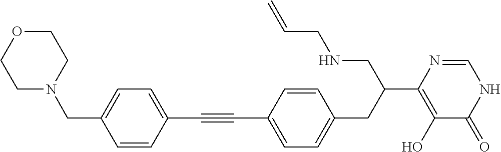 [SMILES: C=CCNCC(Cc1ccc(C#Cc2ccc(CN3CCOCC3)cc2)cc1)c1nc[nH]c(=O)c1O] | 485.25 |
| 57 | 6-(1-((2,2-difluoroethyl) amino)-3-(4-((4- (morpholinomethyl) phenyl)ethynyl)phenyl) propan-2-yl)-5- hydroxypyrimidin- 4(3H)-one | 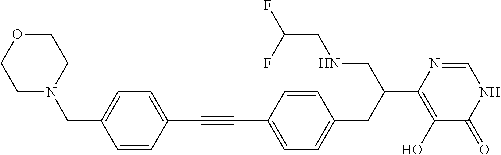 [SMILES: O=c1[nH]cnc(C(CNCC(F)F)Cc2ccc(C#Cc3ccc(CN4CCOCC4)cc3)cc2)c1O] | 509.23 |
| 58 | 6-(1-((2-fluoroethyl) amino)-3-(4-((4- (morpholinomethyl) phenyl)ethynyl)phenyl) propan-2-yl)-5- hydroxypyrimidin- 4(3H)-one | 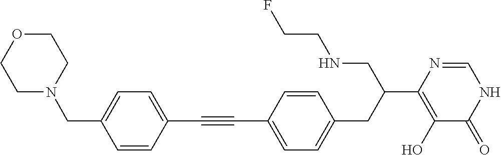 [SMILES: O=c1[nH]cnc(C(CNCCF)Cc2ccc(C#Cc3ccc(CN4CCOCC4)cc3)cc2)c1O] | 491.24 |
| 59 | 6-(3-(allylamino)-1-(4- ((4-(morpholinomethyl) phenyl)ethynyl)phenyl) butan-2-yl)-5- hydroxypyrimidin- 4(3H)-one | 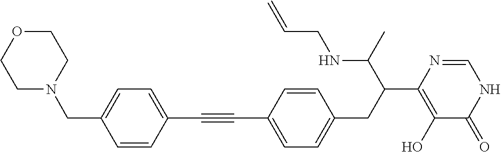 [SMILES: C=CCNC(C)C(Cc1ccc(C#Cc2ccc(CN3CCOCC3)cc2)cc1)c1nc[nH]c(=O)c1O] | 499.23 |
| 60 | 5-hydroxy-6-(1-(4-((4- (morpholinomethyl) phenyl)ethynyl)phenyl)- 3-(propylamino)propan- 2-yl)pyrimidin-4(3H)- one | 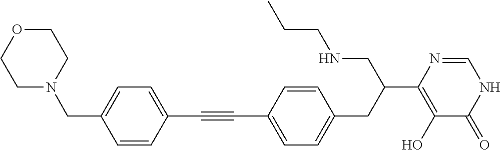 [SMILES: CCCNCC(Cc1ccc(C#Cc2ccc(CN3CCOCC3)cc2)cc1)c1nc[nH]c(=O)c1O] | 487.26 |
| 61 | 6-(1-((2-fluoroethyl) (methyl)amino)-3-(4- ((4-(morpholinomethyl) phenyl)ethynyl)phenyl) propan-2-yl)-5- hydroxypyrimidin- 4(3H)-one | 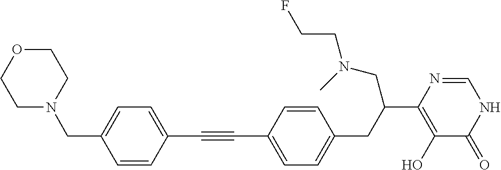 [SMILES: CN(CCF)CC(Cc1ccc(C#Cc2ccc(CN3CCOCC3)cc2)cc1)c1nc[nH]c(=O)c1O] | 505.25 |
| 62 | 6-(1-(cyclobutylamino)- 3-(4-((4- (morpholinomethyl) phenyl)ethynyl)phenyl) propan-2-yl)-5- hydroxypyrimidin- 4(3H)-one | 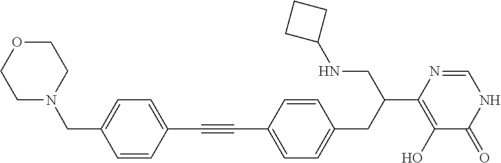 [SMILES: O=c1[nH]cnc(C(CNC2CCC2)Cc2ccc(C#Cc3ccc(CN4CCOCC4)cc3)cc2)c1O] | 499.07 |
| 63 | 6-(1-(4-((4-((4- acetylpiperazin-1-yl) methyl)phenyl)ethynyl) phenyl)-3-(allylamino) propan-2-yl)-5- hydroxypyrimidin- 4(3H)-one | 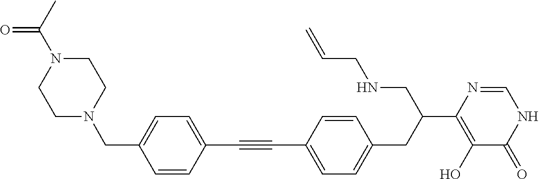 [SMILES: C=CCNCC(Cc1ccc(C#Cc2ccc(CN3CCN(C(C)=O)CC3)cc2)cc1)c1nc[nH]c(=O)c1O] | 526.17 |
| 64 | 5-hydroxy-6-(1-((2- (methylsulfonyl)ethyl) amino)-3-(4-((4- (morpholinomethyl) phenyl)ethynyl)phenyl) propan-2-yl)pyrimidin- 4(3H)-one | 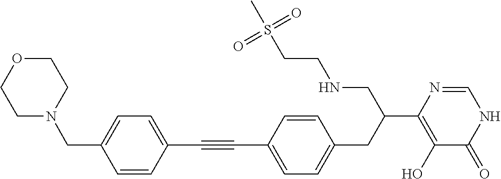 [SMILES: CS(=O)(=O)CCNCC(Cc1ccc(C#Cc2ccc(CN3CCOCC3)cc2)cc1)c1nc[nH]c(=O)c1O] | 551.33 |
| 65 | 5-hydroxy-6-(1-((2- methoxyethyl)amino)-3- (4-((4- (morpholinomethyl) phenyl)ethynyl)phenyl) propan-2-yl)pyrimidin- 4(3H)-one | 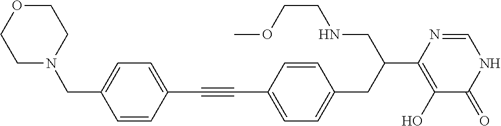 [SMILES: COCCNCC(Cc1ccc(C#Cc2ccc(CN3CCOCC3)cc2)cc1)c1nc[nH]c(=O)c1O] | 503.26 |
| 66 | 6-(1- (cyclopropylamino)- 3-(4-((4- (morpholinomethyl) phenyl)ethynyl)phenyl) propan-2-yl)-5- hydroxypyrimidin- 4(3H)-one | 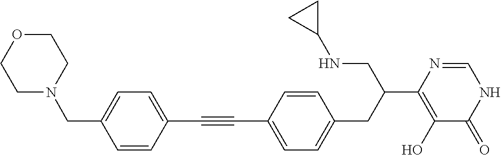 [SMILES: O=c1[nH]cnc(C(CNC2CC2)Cc2ccc(C#Cc3ccc(CN4CCOCC4)cc3)cc2)c1O] | 485.25 |
| 67 | 6-(1- (cyclopentylamino)- 3-(4-((4- (morpholinomethyl) phenyl)ethynyl)phenyl) propan-2-yl)-5- hydroxypyrimidin- 4(3H)-one | 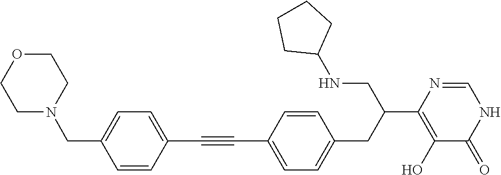 [SMILES: O=c1[nH]cnc(C(CNC2CCCC2)Cc2ccc(C#Cc3ccc(CN4CCOCC4)cc3)cc2)c1O] | 513.18 |
| 68 | 5-hydroxy-6-(1- (methylamino)- 3-(4-((4- (morpholinomethyl) phenyl)ethynyl) phenyl)propan- 2-yl)pyrimidin- 4(3H)-one | 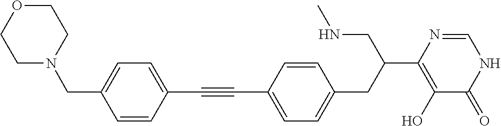 [SMILES: CNCC(Cc1ccc(C#Cc2ccc(CN3CCOCC3)cc2)cc1)c1nc[nH]c(=O)c1O] | 459.23 |
| 69 | 5-hydroxy-6-(1-((2- hydroxyethyl)amino)- 3-(4-((4- (morpholinomethyl) phenyl)ethynyl)phenyl) propan-2-yl)pyrimidin- 4(3H)-one | 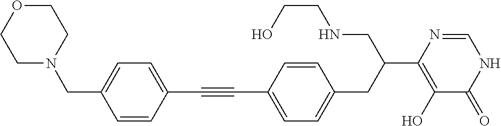 [SMILES: O=c1[nH]cnc(C(CNCCO)Cc2ccc(C#Cc3ccc(CN4CCOCC4)cc3)cc2)c1O] | 489.14 |
| 70 | 5-hydroxy-6-(1-(3- hydroxyazetidin-1-yl)- 3-(4-((4- (morpholinomethyl) phenyl)ethynyl)phenyl) propan-2-yl)pyrimidin- 4(3H)-one | 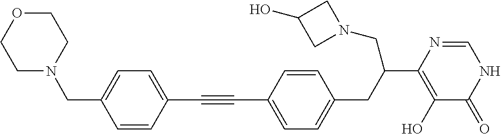 [SMILES: O=c1[nH]cnc(C(Cc2ccc(C#Cc3ccc(CN4CCOCC4)cc3)cc2)CN2CC(O)C2)c1O] | 501.15 |
| 71 | 6-(1-(dimethylamino)- 3-(4-((4- (morpholinomethyl) phenyl)ethynyl)phenyl) propan-2-yl)-5- hydroxypyrimidin- 4(3H)-one | 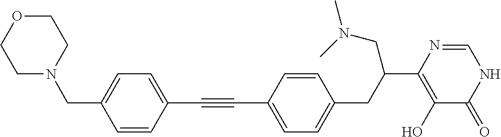 [SMILES: CN(C)CC(Cc1ccc(C#Cc2ccc(CN3CCOCC3)cc2)cc1)c1nc[nH]c(=O)c1O] | 473.25 |
| 72 | 6-(1-(4-((4-((1,1- dioxidothiomorpholino) methyl)phenyl)ethynyl) phenyl)-3-(vinylamino) propan-2-yl)-5- hydroxypyrimidin- 4(3H)-one | 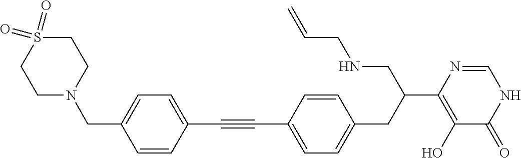 [SMILES: C=CCNCC(Cc1ccc(C#Cc2ccc(CN3CCS(=O)(=O)CC3)cc2)cc1)c1nc[nH]c(=O)c1O] | 519.21 |
| 73 | 6-(1-amino-3-(4-((4- (morpholinomethyl) phenyl)ethynyl)phenyl) propan-2-yl)-5- hydroxypyrimidin- 4(3H)-one | 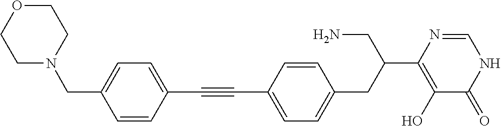 [SMILES: NCC(Cc1ccc(C#Cc2ccc(CN3CCOCC3)cc2)cc1)c1nc[nH]c(=O)c1O] | 445.12 |
| 74 | 5-hydroxy-6-(1-((2- (methylamino)ethyl) amino)-3-(4-((4- (morpholinomethyl) phenyl)ethynyl)phenyl) propan-2-yl)pyrimidin- 4(3H)-one | 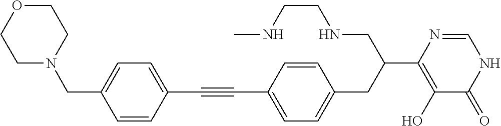 [SMILES: CNCCNCC(Cc1ccc(C#Cc2ccc(CN3CCOCC3)cc2)cc1)c1nc[nH]c(=O)c1O] | 502.27 |
| 75 | 6-(3-amino-1-(4-((4- (morpholinomethyl) phenyl)ethynyl)phenyl) butan-2-yl)-5- hydroxypyrimidin- 4(3H)-one | 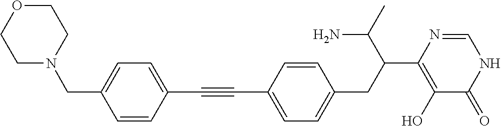 [SMILES: CC(N)C(Cc1ccc(C#Cc2ccc(CN3CCOCC3)cc2)cc1)c1nc[nH]c(=O)c1O] | 459.23 |
| 76 | 5-hydroxy-6-(1-(((R)- 1-hydroxybut-3-en-2- yl)amino)-3-(4-((4- (morpholinomethyl) phenyl)ethynyl)phenyl) propan-2-yl)pyrimidin- 4(3H)-one | 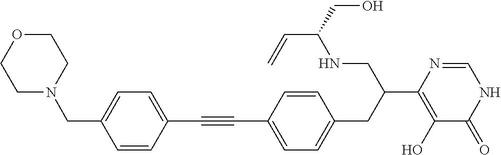 [SMILES: C=C[C@H](CO)NCC(Cc1ccc(C#Cc2ccc(CN3CCOCC3)cc2)cc1)c1nc[nH]c(=O)c1O] | 515.03 |
| 77 | 5-hydroxy-6-(1-((3- methyloxetan-3-yl) amino)-3-(4-((4- (morpholinomethyl) phenyl)ethynyl)phenyl) propan-2-yl)pyrimidin- 4(3H)-one | 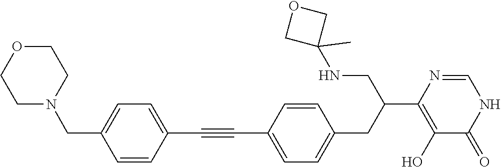 [SMILES: CC1(NCC(Cc2ccc(C#Cc3ccc(CN4CCOCC4)cc3)cc2)c2nc[nH]c(=O)c2O)COC1] | 515.26 |
| 78 | 6-(1-((2-fluoroethyl) amino)-3-(4-((4- (((2-methoxyethyl) (methyl)amino)methyl) phenyl)ethynyl)phenyl) propan-2-yl)-5- hydroxypyrimidin- 4(3H)-one | 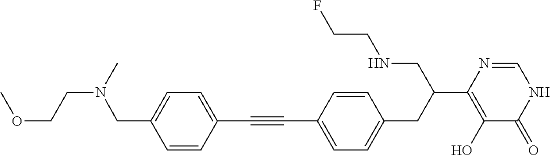 [SMILES: COCCN(C)Cc1ccc(C#Cc2ccc(CC(CNCCF)c3nc[nH]c(=O)c3O)cc2)cc1] | 593.25 |
| 79 | 6-(1-(4-((4-(((2S,6S)- 2,6-dimethylmorpholino) methyl)phenyl)ethynyl) phenyl)-3-((2-fluoroethyl) amino)propan-2-yl)-5- hydroxypyrimidin-4(3H)- one | 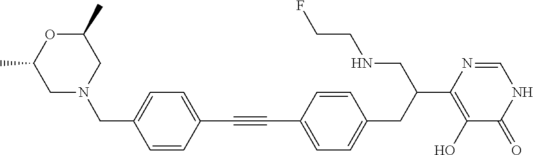 [SMILES: C[C@H]1CN(Cc2ccc(C#Cc3ccc(CC(CNCCF)c4nc[nH]c(=O)c4O)cc3)cc2)C[C@H](C)O1] | 519.23 |
| 80 | N-(2-(5-hydroxy-6-oxo- 1,6-dihydropyrimidin- 4-yl)-3-(4-((4- (morpholinomethyl) phenyl)ethynyl)phenyl) propyl) methanesulfonamide | 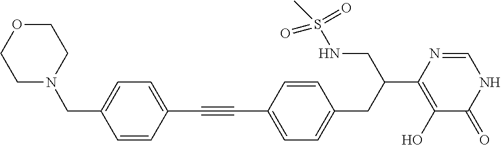 [SMILES: CS(=O)(=O)NCC(Cc1ccc(C#Cc2ccc(CN3CCOCC3)cc2)cc1)c1nc[nH]c(=O)c1O] | 523.19 |
| 81 | N-(2-(5-hydroxy-6-oxo- 1,6-dihydropyrimidin- 4-yl)-3-(4-((4- (morpholinomethyl) phenyl)ethynyl)phenyl) propyl) ethanesulfonamide | 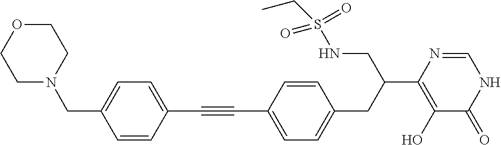 [SMILES: CCS(=O)(=O)NCC(Cc1ccc(C#Cc2ccc(CN3CCOCC3)cc2)cc1)c1nc[nH]c(=O)c1O] | 537.21 |
| 82 | 1,1-difluoro-N-(2-(5- hydroxy-6-oxo-1,6- dihydropyrimidin- 4-yl)-3-(4-((4- (morpholinomethyl) phenyl)ethynyl)phenyl) propyl) methanesulfonamide | 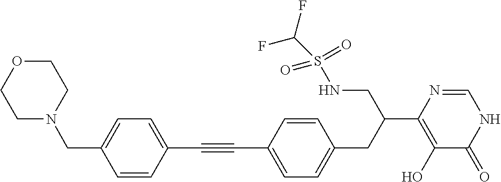 [SMILES: O=c1[nH]cnc(C(CNS(=O)(=O)C(F)F)Cc2ccc(C#Cc3ccc(CN4CCOCC4)cc3)cc2)c1O] | 559.17 |
| 83 | N-(2-(5-hydroxy-6-oxo- 1,6-dihydropyrimidin- 4-yl)-3-(4-((4- (morpholinomethyl) phenyl)ethynyl)phenyl) propyl) cyclopropanesulfonamide | 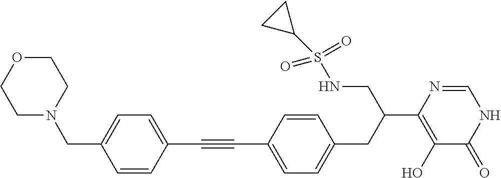 [SMILES: O=c1[nH]cnc(C(CNS(=O)(=O)C2CC2)Cc2ccc(C#Cc3ccc(CN4CCOCC4)cc3)cc2)c1O] | 549.21 |
| 84 | 2-(5-hydroxy-6-oxo- 1,6-dihydropyrimidin- 4-yl)-3-(4-((4- (morpholinomethyl) phenyl)ethynyl)phenyl) propyl methanesulfonate | 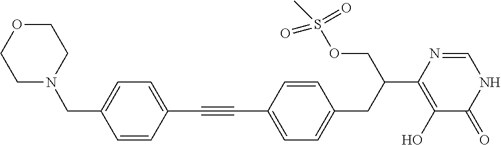 [SMILES: CS(=O)(=O)OCC(Cc1ccc(C#Cc2ccc(CN3CCOCC3)cc2)cc1)c1nc[nH]c(=O)c1O] | 524.18 |
| 85 | methyl (2-(5-hydroxy- 6-oxo-1,6- dihydropyrimidin-4- yl)-3-(4-((4- (morpholinomethyl) phenyl)ethynyl)phenyl) propyl)sulfamide | 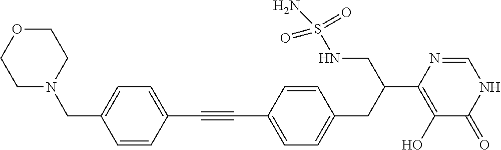 [SMILES: NS(=O)(=O)NCC(Cc1ccc(C#Cc2ccc(CN3CCOCC3)cc2)cc1)c1nc[nH]c(=O)c1O] | 538.21 |
| 86 | 5-hydroxy-6-(1-hydroxy- 3-(4-((4- (morpholinomethyl) phenyl)ethynyl)phenyl)- 1-phenylpropan-2- yl)pyrimidin-4(3H)-one | 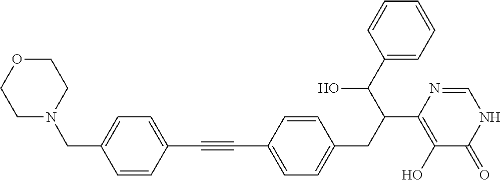 [SMILES: O=c1[nH]cnc(C(Cc2ccc(C#Cc3ccc(CN4CCOCC4)cc3)cc2)C(O)c2ccccc2)c1O] | 522.21 |
| 87 | 5-hydroxy-6-(1-((5- methyl-1,3,4-oxadiazol- 2-yl)amino)-3-(4-((4- (morpholinomethyl) phenyl)ethynyl)phenyl) propan-2-yl)pyrimidin- 4(3H)-one | 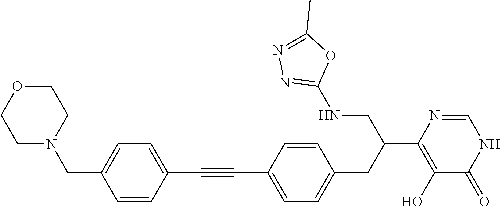 [SMILES: Cc1nnc(NCC(Cc2ccc(C#Cc3ccc(CN4CCOCC4)cc3)cc2)c2nc[nH]c(=O)c2O)o1] | 527.23 |
| 88 | 5-hydroxy-6-(1- (isoxazol-5-ylamino)- 3-(4-((4- (morpholinomethyl) phenyl)ethynyl)phenyl) propan-2-yl)pyrimidin- 4(3H)-one | 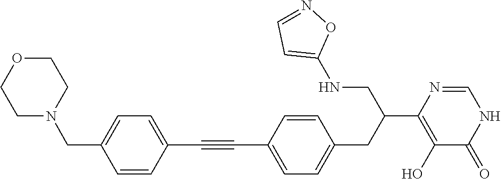 [SMILES: O=c1[nH]cnc(C(CNc2ccno2)Cc2ccc(C#Cc3ccc(CN4CCOCC4)cc3)cc2)c1O] | 512.23 |
| 89 | 6-(1-((1H-tetrazol-5- yl)amino)-3-(4-((4- (morpholinomethyl) phenyl)ethynyl)phenyl) propan-2-yl)-5- hydroxypyrimidin- 4(3H)-one | 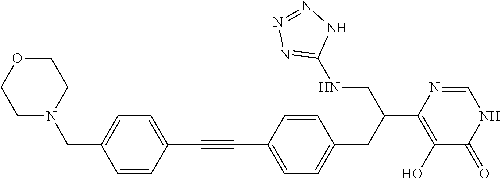 [SMILES: O=c1[nH]cnc(C(CNc2nnn[nH]2)Cc2ccc(C#Cc3ccc(CN4CCOCC4)cc3)cc2)c1O] | 513.12 |
| 90 | 6-(1-((1H-pyrazol-4- yl)amino)-3-(4-((4- (morpholinomethyl) phenyl)ethynyl)phenyl) propan-2-yl)-5- hydroxypyrimidin- 4(3H)-one | 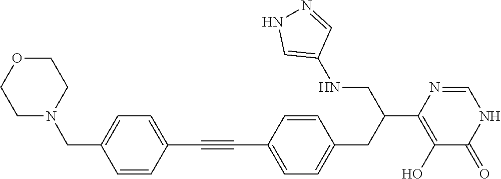 [SMILES: O=c1[nH]cnc(C(CNc2cn[nH]c2)Cc2ccc(C#Cc3ccc(CN4CCOCC4)cc3)cc2)c1O] | 511.24 |
| 91 | 5-hydroxy-6-(1-(4- ((4-(morpholinomethyl) phenyl)ethynyl)phenyl)- 3-oxobutan-2- yl)pyrimidin-4(3H)-one | 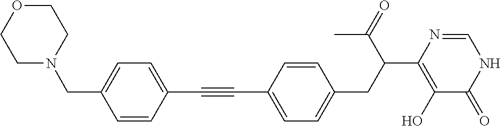 [SMILES: CC(=O)C(Cc1ccc(C#Cc2ccc(CN3CCOCC3)cc2)cc1)c1nc[nH]c(=O)c1O] | 458.20 |
| 92 | 5-hydroxy-6-(3-(4-((4- (morpholinomethyl) phenyl)ethynyl)phenyl)- 1-oxo-1-phenylpropan- 2-yl)pyrimidin-4(3H)- one | 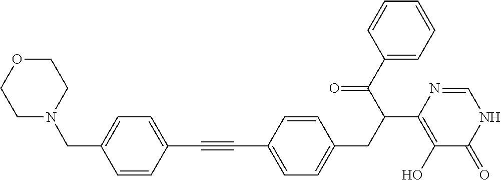 [SMILES: O=C(c1ccccc1)C(Cc1ccc(C#Cc2ccc(CN3CCOCC3)cc2)cc1)c1nc[nH]c(=O)c1O] | 520.22 |
| 93 | 5-hydroxy-6-(2-(4-((4- (morpholinomethyl) phenyl)ethynyl)phenyl)- 1-(1H-pyrazol-5- yl)ethyl)pyrimidin- 4(3H)-one | 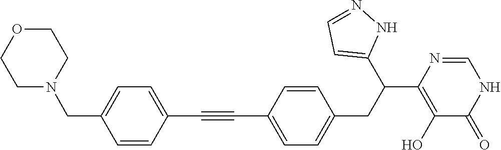 [SMILES: O=c1[nH]cnc(C(Cc2ccc(C#Cc3ccc(CN4CCOCC4)cc3)cc2)c2ccn[nH]2)c1O] | 482.21 |
| 94 | 5-hydroxy-6-(1- (isoxazol-5-yl)-2-(4- ((4-(morpholinomethyl) phenyl)ethynyl)phenyl) ethyl)pyrimidin- 4(3H)-one | 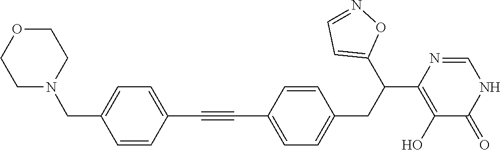 [SMILES: O=c1[nH]cnc(C(Cc2ccc(C#Cc3ccc(CN4CCOCC4)cc3)cc2)c2ccno2)c1O] | 483.11 |
| 95 | 5-hydroxy-6-(2-(4-((4- (morpholinomethyl) phenyl)ethynyl)phenyl)- 1-(1H-tetrazol-5- yl)ethyl)pyrimidin- 4(3H)-one | 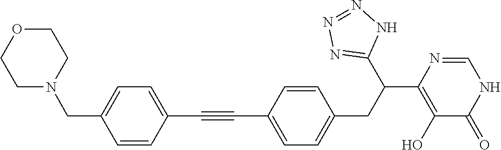 [SMILES: O=c1[nH]cnc(C(Cc2ccc(C#Cc3ccc(CN4CCOCC4)cc3)cc2)c2nnn[nH]2)c1O] | 484.20 |
| 96 | 6-(1-(5-amino-1H- tetrazol-1-yl)-2-(4- ((4- (morpholinomethyl) phenyl)ethynyl) phenyl)ethyl)-5- hydroxypyrimidin- 4(3H)-one | 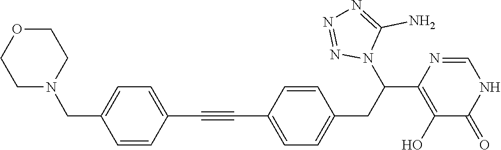 [SMILES: Nc1nnnn1C(Cc1ccc(C#Cc2ccc(CN3CCOCC3)cc2)cc1)c1nc[nH]c(=O)c1O] | 499.21 |
| 97 | 5-hydroxy-6-(3-(4- ((4-(morpholinomethyl) phenyl)ethynyl)phenyl) prop-1-en-2-yl)pyrimidin- 4(3H)-one | 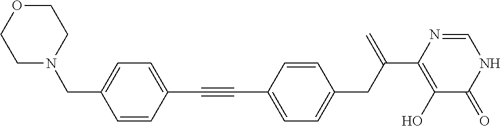 [SMILES: C=C(Cc1ccc(C#Cc2ccc(CN3CCOCC3)cc2)cc1)c1nc[nH]c(=O)c1O] | 428.19 |
| 98 | 6-(3-fluoro-4-((4- (morpholinomethyl) phenyl)ethynyl) phenethyl)-5- hydroxypyrimidin- 4(3H)-one | 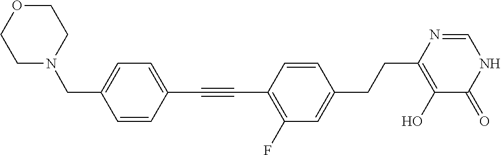 [SMILES: O=c1[nH]cnc(CCc2ccc(C#Cc3ccc(CN4CCOCC4)cc3)c(F)c2)c1O] | 434.18 |
| 99 | 6-(1-((2-fluoroethyl) amino)-3-(4-((4- (((R)-3- methoxypyrrolidin- 1-yl)methyl)phenyl) ethynyl)phenyl)propan- 2-yl)-5- hydroxypyrimidin- 4(3H)-one | 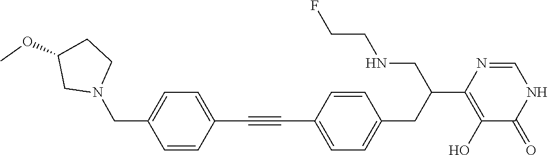 [SMILES: CO[C@@H]1CCN(Cc2ccc(C#Cc3ccc(CC(CNCCF)c4nc[nH]c(=O)c4O)cc3)cc2)C1] | 505.26 |
| 100 | 5-hydroxy-6-(1- ((isoxazol-3- ylmethyl)amino)-3- (4-((4- (morpholinomethyl) phenyl)ethynyl)phenyl) propan-2-yl)pyrimidin- 4(3H)-one | 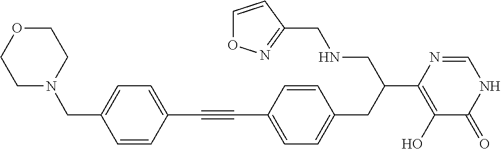 [SMILES: O=c1[nH]cnc(C(CNCc2ccon2)Cc2ccc(C#Cc3ccc(CN4CCOCC4)cc3)cc2)c1O] | 526.24 |
| 101 | 3-(4-((4-((1,3- oxazetidin-3- yl)methyl)phenyl) ethynyl)phenyl)-N- (2,2-difluoroethyl)- 2-(5-hydroxy-6-oxo- 1,6-dihydropyrimidin- 4-yl)propanamide | 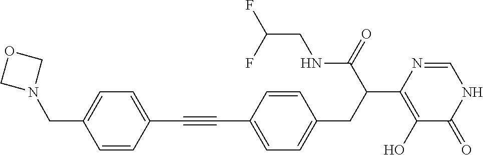 [SMILES: O=C(NCC(F)F)C(Cc1ccc(C#Cc2ccc(CN3COC3)cc2)cc1)c1nc[nH]c(=O)c1O] | 495.17 |
| 102 | 3-(4-((4-((3- cyanopyrrolidin-1- yl)methyl)phenyl) ethynyl)phenyl)-N- (2,2-difluoroethyl)- 2-(5-hydroxy-6-oxo- 1,6-dihydropyrimidin- 4-yl)propanamide | 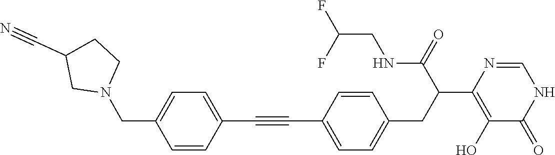 [SMILES: N#CC1CCN(Cc2ccc(C#Cc3ccc(CC(C(=O)NCC(F)F)c4nc[nH]c(=O)c4O)cc3)cc2)C1] | 532.21 |
| 103 | N-(2,2-difluoroethyl)- 3-(3-fluoro-4-((4- (morpholinomethyl) phenyl)ethynyl)phenyl)- 2-(5-hydroxy-6-oxo-1,6- dihydropyrimidin-4- yl)propanamide | 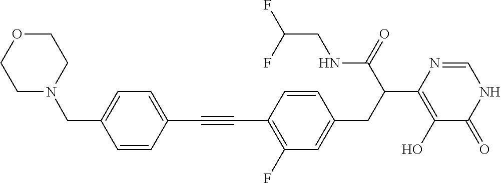 [SMILES: O=C(NCC(F)F)C(Cc1ccc(C#Cc2ccc(CN3CCOCC3)cc2)c(F)c1)c1nc[nH]c(=O)c1O] | 541.20 |
| 104 | 3-(4-((4-((4- cyanopiperidin-1- yl)methyl)phenyl) ethynyl)phenyl)-N- (2,2-difluoroethyl)- 2-(5-hydroxy-6-oxo- 1,6-dihydropyrimidin- 4-yl)propanamide | 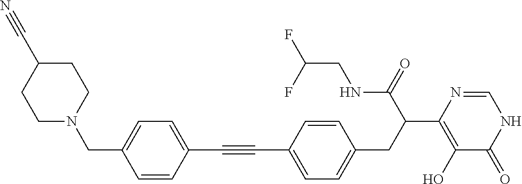 [SMILES: N#CC1CCN(Cc2ccc(C#Cc3ccc(CC(C(=O)NCC(F)F)c4nc[nH]c(=O)c4O)cc3)cc2)CC1] | 546.23 |
| 105 | N-(2,2-difluoroethyl)- 2-(5-hydroxy-6-oxo- 1,6-dihydropyrimidin- 4-yl)-3-(4-((4-(((S)-3- methoxypyrrolidin- 1-yl)methyl)phenyl) ethynyl)phenyl) propanamide | 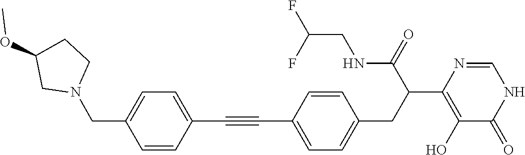 [SMILES: CO[C@H]1CCN(Cc2ccc(C#Cc3ccc(CC(C(=O)NCC(F)F)c4nc[nH]c(=O)c4O)cc3)cc2)C1] | 537.13 |
| 106 | N-(2,2-difluoroethyl)- 2-(5-hydroxy-6-oxo- 1,6-dihydropyrimidin- 4-yl)-3-(4-((4-(((R)-3- methoxypyrrolidin- 1-yl)methyl)phenyl) ethynyl)phenyl) propanamide | 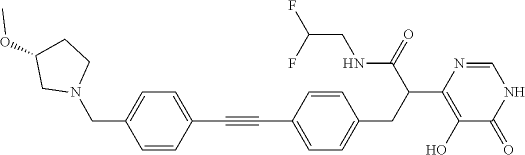 [SMILES: CO[C@@H]1CCN(Cc2ccc(C#Cc3ccc(CC(C(=O)NCC(F)F)c4nc[nH]c(=O)c4O)cc3)cc2)C1] | 537.32 |
| 107 | 3-(4-((4-(((R)-3- cyanopyrrolidin-1- yl)methyl)phenyl) ethynyl)phenyl)-N- (2,2-difluoroethyl)- 2-(5-hydroxy-6-oxo- 1,6-dihydropyrimidin- 4-yl)propanamide | 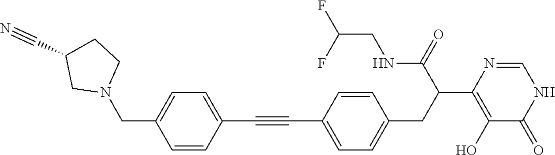 [SMILES: N#C[C@@H]1CCN(Cc2ccc(C#Cc3ccc(CC(C(=O)NCC(F)F)c4nc[nH]c(=O)c4O)cc3)cc2)C1] | 532.21 |
| 108 | N-(2,2-difluoroethyl)- 2-(5-hydroxy-6-oxo- 1,6-dihydropyrimidin- 4-yl)-3-(4-((4- (hydroxymethyl) phenyl)ethynyl)phenyl) propanamide | 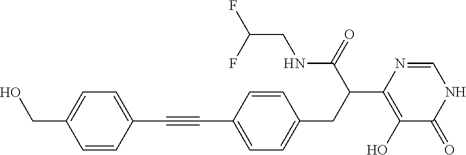 [SMILES: O=C(NCC(F)F)C(Cc1ccc(C#Cc2ccc(CO)cc2)cc1)c1nc[nH]c(=O)c1O] | 544.15 |
| 109 | N-(2,2-difluoroethyl)- 2-(5-hydroxy-6-oxo- 1,6-dihydropyrimidin- 4-yl)-3-(4-((4-((3- methoxyazetidin-1- yl)methyl)phenyl) ethynyl)phenyl) propanamide | 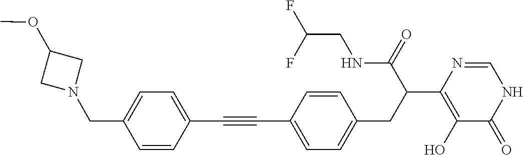 [SMILES: COC1CN(Cc2ccc(C#Cc3ccc(CC(C(=O)NCC(F)F)c4nc[nH]c(=O)c4O)cc3)cc2)C1] | 523.21 |
| 110 | N-(2,2-difluoroethyl)- 2-(5-hydroxy-6-oxo- 1,6-dihydropyrimidin- 4-yl)-3-(4-((4-(((2- methoxyethyl)(methyl) amino)methyl)phenyl) ethynyl)phenyl) propanamide | 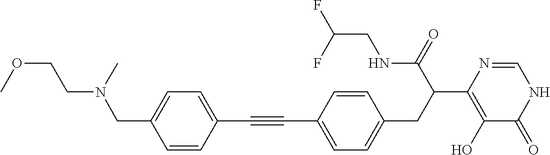 [SMILES: COCCN(C)Cc1ccc(C#Cc2ccc(CC(C(=O)NCC(F)F)c3nc[nH]c(=O)c3O)cc2)cc1] | 525.33 |
| 111 | N-(2,2-difluoroethyl)- 2-(5-hydroxy-6-oxo- 1,6-dihydropyrimidin- 4-yl)-3-(4-((4-((2- oxopyrrolidin-1- yl)methyl)phenyl) ethynyl)phenyl) propanamide | 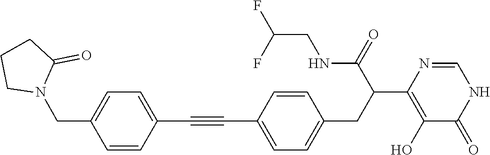 [SMILES: O=C(NCC(F)F)C(Cc1ccc(C#Cc2ccc(CN3CCCC3=O)cc2)cc1)c1nc[nH]c(=O)c1O] | 521.10 |
| 112 | N-(2,2-difluoroethyl)- 2-(5-hydroxy-6-oxo- 1,6-dihydropyrimidin- 4-yl)-3-(4-((4- ((methyl(oxetan-3- yl)amino)methyl) phenyl)ethynyl)phenyl) propanamide | 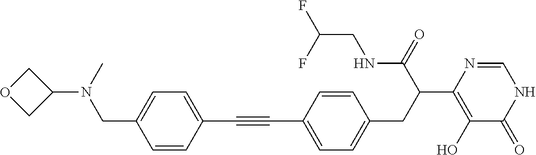 [SMILES: CN(Cc1ccc(C#Cc2ccc(CC(C(=O)NCC(F)F)c3nc[nH]c(=O)c3O)cc2)cc1)C1COC1] | 523.41 |
| 113 | N-(2,2-difluoroethyl)- 2-(5-hydroxy-6-oxo- 1,6-dihydropyrimidin- 4-yl)-3-(4-((4-(2- hydroxyethyl)phenyl) ethynyl)phenyl) propanamide | 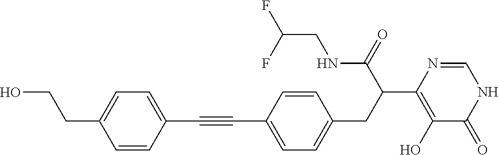 [SMILES: O=C(NCC(F)F)C(Cc1ccc(C#Cc2ccc(CCO)cc2)cc1)c1nc[nH]c(=O)c1O] | 468.17 |
| 114 | N-(2,2-difluoroethyl)- 2-(5-hydroxy-6-oxo- 1,6-dihydropyrimidin- 4-yl)-3-(4-((4-(2- methoxyethyl)phenyl) ethynyl)phenyl) propanamide | 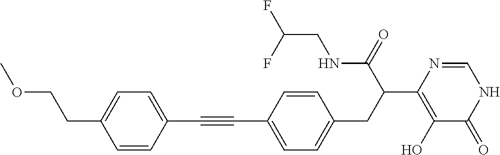 [SMILES: COCCc1ccc(C#Cc2ccc(CC(C(=O)NCC(F)F)c3nc[nH]c(=O)c3O)cc2)cc1] | 482.20 |
| 115 | 3-(4-((4-((4- acetylpiperazin-1- yl)methyl)phenyl) ethynyl)phenyl)-N- (2,2-difluoroethyl)- 2-(5-hydroxy-6-oxo- 1,6-dihydropyrimidin- 4-yl)propanamide | 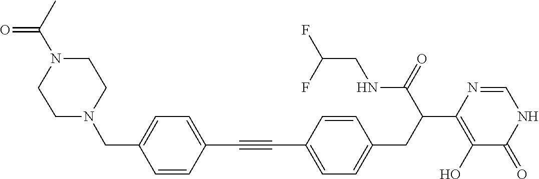 [SMILES: CC(=O)N1CCN(Cc2ccc(C#Cc3ccc(CC(C(=O)NCC(F)F)c4nc[nH]c(=O)c4O)cc3)cc2)CC1] | 564.34 |
| 116 | 3-(4-((4-((bis(2- methoxyethyl)amino) methyl)phenyl)ethynyl) phenyl)-N-(2,2- difluoroethyl)-2-(5- hydroxy-6-oxo-1,6- dihydropyrimidin-4- yl)propanamide | 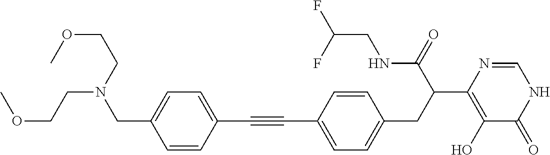 [SMILES: COCCN(CCOC)Cc1ccc(C#Cc2ccc(CC(C(=O)NCC(F)F)c3nc[nH]c(=O)c3O)cc2)cc1] | 569.23 |
| 117 | 3-(4-((4-((3- (chloromethyl)-3- (hydroxymethyl) azetidin-1- yl)methyl)phenyl) ethynyl)phenyl)-N- (2,2-difluoroethyl)- 2-(5-hydroxy-6-oxo- 1,6-dihydropyrimidin- 4-yl)propanamide | 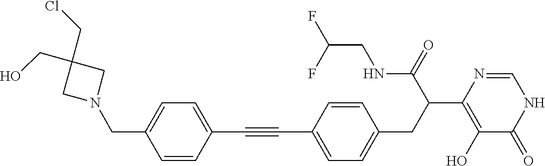 [SMILES: O=C(NCC(F)F)C(Cc1ccc(C#Cc2ccc(CN3CC(CO)(CCl)C3)cc2)cc1)c1nc[nH]c(=O)c1O] | 572.18 |
| 118 | N-(2,2-difluoroethyl)- 3-(4-((4-((2,2- dimethylmorpholino) methyl)phenyl)ethynyl) phenyl)-2-(5-hydroxy- 6-oxo-1,6- dihydropyrimidin-4- yl)propanamide | 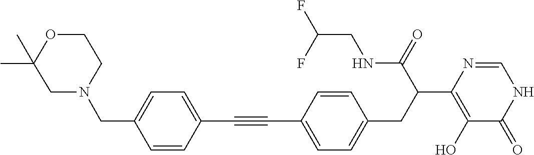 [SMILES: CC1(C)CN(Cc2ccc(C#Cc3ccc(CC(C(=O)NCC(F)F)c4nc[nH]c(=O)c4O)cc3)cc2)CCO1] | 551.14 |
| 119 | 3-(4-((4- ((cyclopentylamino) methyl)phenyl)ethynyl) phenyl)-N-(2,2- difluoroethyl)-2-(5- hydroxy-6-oxo-1,6- dihydropyrimidin-4- yl)propanamide | 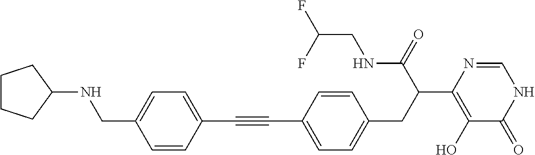 [SMILES: O=C(NCC(F)F)C(Cc1ccc(C#Cc2ccc(CNC3CCCC3)cc2)cc1)c1nc[nH]c(=O)c1O] | 521.31 |
| 120 | N-(2,2-difluoroethyl)- 2-(5-hydroxy-6-oxo- 1,6-dihydropyrimidin- 4-yl)-3-(4-((4- (morpholine-4- carbonyl)phenyl) ethynyl)phenyl) propanamide | 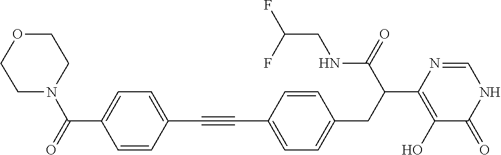 [SMILES: O=C(NCC(F)F)C(Cc1ccc(C#Cc2ccc(C(=O)N3CCOCC3)cc2)cc1)c1nc[nH]c(=O)c1O] | 537.29 |
| 121 | N-(2,2-difluoroethyl)- 2-(5-hydroxy-6-oxo- 1,6-dihydropyrimidin- 4-yl)-3-(4-((4-(2- ((2-methoxyethyl) (methyl)amino)ethyl) phenyl)ethynyl)phenyl) propanamide | 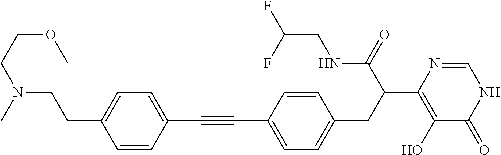 [SMILES: COCCN(C)CCc1ccc(C#Cc2ccc(CC(C(=O)NCC(F)F)c3nc[nH]c(=O)c3O)cc2)cc1] | 539.34 |
| 122 | 3-(4-((4-(2-(4- acetylpiperazin-1- yl)ethyl)phenyl) ethynyl)phenyl)-N- (2,2-difluoroethyl)- 2-(5-hydroxy-6-oxo- 1,6-dihydropyrimidin- 4-yl)propanamide | 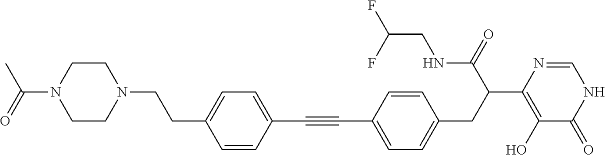 [SMILES: CC(=O)N1CCN(CCc2ccc(C#Cc3ccc(CC(C(=O)NCC(F)F)c4nc[nH]c(=O)c4O)cc3)cc2)CC1] | 578.15 |
| 123 | 3-(4-((4-((6- (chloromethyl)-3- azabicyclo[3.1.0]hexan- 3-yl)methyl)phenyl) ethynyl)phenyl)-N- (2,2-difluoroethyl)- 2-(5-hydroxy-6-oxo- 1,6-dihydropyrimidin- 4-yl)propanamide | 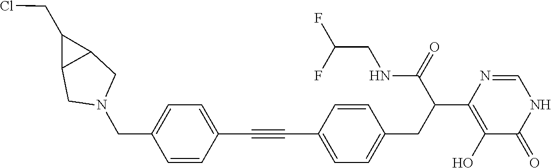 [SMILES: O=C(NCC(F)F)C(Cc1ccc(C#Cc2ccc(CN3CC4C(CCl)C4C3)cc2)cc1)c1nc[nH]c(=O)c1O] | 567.18 |
| 124 | N-(2,2-difluoroethyl)- 2-(5-hydroxy-6-oxo- 1,6-dihydropyrimidin- 4-yl)-3-(4-((4-(2- (piperazin-1- yl)ethyl)phenyl) ethynyl)phenyl) propanamide | 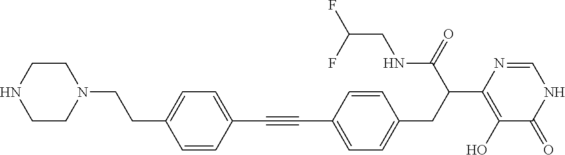 [SMILES: O=C(NCC(F)F)C(Cc1ccc(C#Cc2ccc(CCN3CCNCC3)cc2)cc1)c1nc[nH]c(=O)c1O] | 536.14 |
| 125 | N-(2,2-difluoroethyl)- 2-(5-hydroxy-6-oxo- 1,6-dihydropyrimidin- 4-yl)-3-(4-((4-(2-(3- methoxyazetidin-1- yl)ethyl)phenyl) ethynyl)phenyl) propanamide | 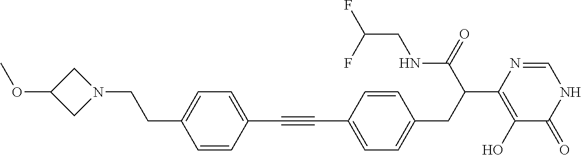 [SMILES: COC1CN(CCc2ccc(C#Cc3ccc(CC(C(=O)NCC(F)F)c4nc[nH]c(=O)c4O)cc3)cc2)C1] | 537.33 |
| 126 | N-(2,2-difluoroethyl)- 2-(5-hydroxy-6-oxo- 1,6-dihydropyrimidin- 4-yl)-3-(4-((3-(2- hydroxyethyl)phenyl) ethynyl)phenyl) propanamide | 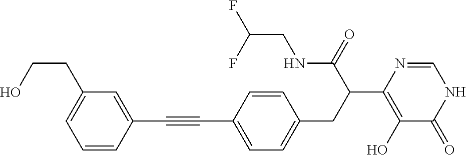 [SMILES: O=C(NCC(F)F)C(Cc1ccc(C#Cc2cccc(CCO)c2)cc1)c1nc[nH]c(=O)c1O] | 468.22 |
| 127 | 3-(4-((4-(2- aminoethyl)phenyl) ethynyl)phenyl)-N- (2,2-difluoroethyl)- 2-(5-hydroxy-6-oxo- 1,6-dihydropyrimidin- 4-yl)propanamide | 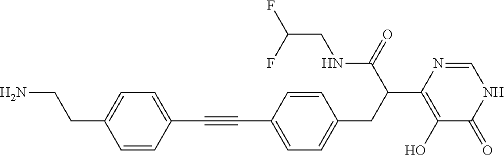 [SMILES: NCCc1ccc(C#Cc2ccc(CC(C(=O)NCC(F)F)c3nc[nH]c(=O)c3O)cc2)cc1] | 467.19 |
| 128 | N-(1,3- dihydroxypropan- 2-yl)-2-(5-hydroxy-6- oxo-1,6- dihydropyrimidin- 4-yl)-3-(4-((4- (morpholinomethyl) phenyl)ethynyl)phenyl) propanamide | 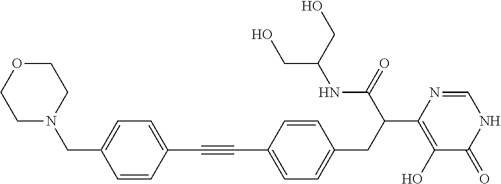 [SMILES: O=C(NC(CO)CO)C(Cc1ccc(C#Cc2ccc(CN3CCOCC3)cc2)cc1)c1nc[nH]c(=O)c1O] | 533.14 |
| 129 | 6-(2-fluoro-4-((4- (morpholinomethyl) phenyl)ethynyl) phenethyl)-5- hydroxypyrimidin- 4(3H)-one | 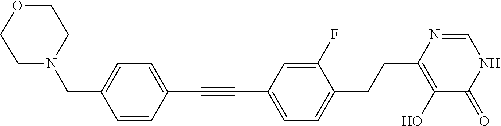 [SMILES: O=c1[nH]cnc(CCc2ccc(C#Cc3ccc(CN4CCOCC4)cc3)cc2F)c1O] | 434.18 |
| 130 | 5-hydroxy-6-(3- methyl-4-((4- (morpholinomethyl) phenyl)ethynyl) phenethyl)pyrimidin- 4(3H)-one | 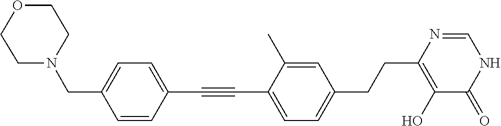 [SMILES: Cc1cc(CCc2nc[nH]c(=O)c2O)ccc1C#Cc1ccc(CN2CCOCC2)cc1] | 430.31 |
| 131 | 6-(1-(3,3- difluoroazetidin-1- yl)-3-(4-((4- (morpholinomethyl) phenyl)ethynyl)phenyl)- 1-oxopropan-2-yl)-5- hydroxypyrimidin- 4(3H)-one | 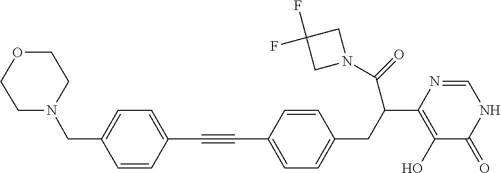 [SMILES: O=C(C(Cc1ccc(C#Cc2ccc(CN3CCOCC3)cc2)cc1)c1nc[nH]c(=O)c1O)N1CC(F)(F)C1] | 535.31 |
| 132 | 2-(5-hydroxy-6-oxo- 1,6-dihydropyrimidin- 4-yl)-N,N-dimethyl-3- (4-((4- (morpholinomethyl) phenyl)ethynyl)phenyl) propanamide | 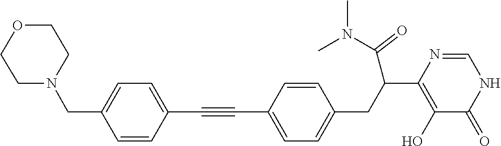 [SMILES: CN(C)C(=O)C(Cc1ccc(C#Cc2ccc(CN3CCOCC3)cc2)cc1)c1nc[nH]c(=O)c1O] | 487.27 |
| 133 | 2-fluoro-N-(2-(5- hydroxy-6-oxo-1,6- dihydropyrimidin-4- yl)-3-(4-((4-((3- methoxyazetidin-1- yl)methyl)phenyl) ethynyl)phenyl)propyl) acetamide | 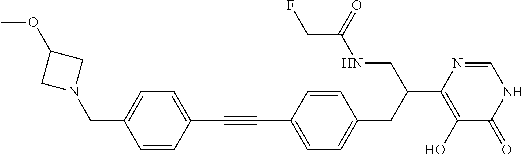 [SMILES: COC1CN(Cc2ccc(C#Cc3ccc(CC(CNC(=O)CF)c4nc[nH]c(=O)c4O)cc3)cc2)C1] | 505.26 |
| 134 | N-(2-(5-hydroxy-6-oxo- 1,6-dihydropyrimidin- 4-yl)-3-(4-((4-((3- methoxyazetidin-1- yl)methyl)phenyl) ethynyl)phenyl)propyl) acetamide | 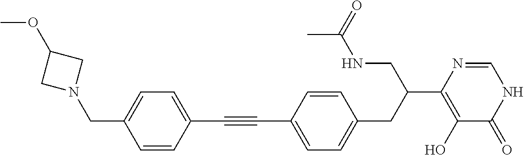 [SMILES: COC1CN(Cc2ccc(C#Cc3ccc(CC(CNC(C)=O)c4nc[nH]c(=O)c4O)cc3)cc2)C1] | 487.12 |
| 135 | N-(2-(5-hydroxy-6- oxo-1,6- dihydropyrimidin-4- yl)-3-(4-((4- (morpholinomethyl) phenyl)ethynyl)phenyl) propyl)tetrahydro-2H- pyran-4-carboxamide | 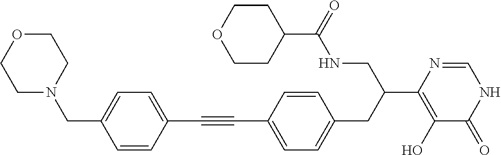 [SMILES: O=C(NCC(Cc1ccc(C#Cc2ccc(CN3CCOCC3)cc2)cc1)c1nc[nH]c(=O)c1O)C1CCOCC1] | 557.17 |
| 136 | N-(3-(4-((4- (chloromethyl) phenyl)ethynyl)phenyl)- 2-(5-hydroxy-6-oxo-1,6- dihydropyrimidin-4- yl)propyl)acetamide | 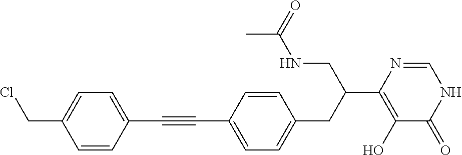 [SMILES: CC(=O)NCC(Cc1ccc(C#Cc2ccc(CCl)cc2)cc1)c1nc[nH]c(=O)c1O] | 437.14 |
| 137 | N-(2-(5-hydroxy-6-oxo- 1,6-dihydropyrimidin- 4-yl)-3-(4-((4-(((5- methylisoxazol-3- yl)amino)methyl)phenyl) ethynyl)phenyl)propyl) acetamide | 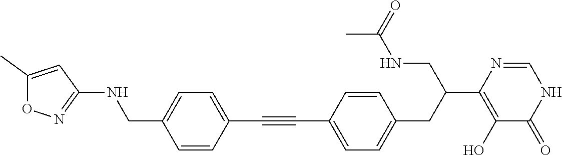 [SMILES: CC(=O)NCC(Cc1ccc(C#Cc2ccc(CNc3cc(C)on3)cc2)cc1)c1nc[nH]c(=O)c1O] | 498.21 |
| 138 | N-(2-(5-hydroxy-6-oxo- 1,6-dihydropyrimidin- 4-yl)-3-(4-((4- (hydroxymethyl)phenyl) ethynyl)phenyl)propyl) acetamide | 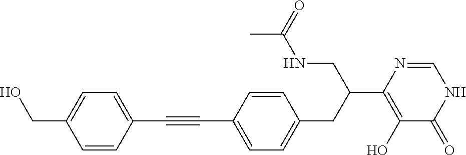 [SMILES: CC(=O)NCC(Cc1ccc(C#Cc2ccc(CO)cc2)cc1)c1nc[nH]c(=O)c1O] | 418.27 |
| 139 | N-(3-(4-((4-((2-oxa-5- azabicyclo[2.2.1]heptan- 5-yl)methyl)phenyl) ethynyl)phenyl)-2-(5- hydroxy-6-oxo-1,6- dihydropyrimidin-4- yl)propyl)acetamide | 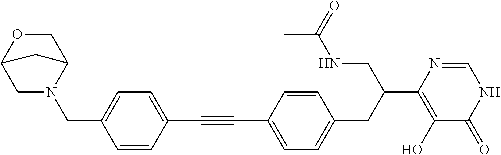 [SMILES: CC(=O)NCC(Cc1ccc(C#Cc2ccc(CN3CC4CC3CO4)cc2)cc1)c1nc[nH]c(=O)c1O] | 499.13 |
| 140 | (E)-4,4,4-trifluoro-N- (2-(5-hydroxy-6-oxo- 1,6-dihydropyrimidin- 4-yl)-3-(4-((4- (morpholinomethyl) phenyl)ethynyl)phenyl) propyl)but-2-enamide | 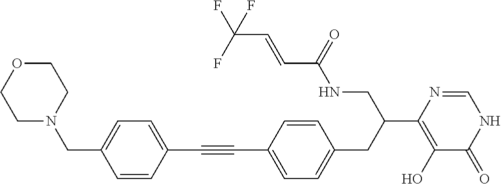 [SMILES: O=C(/C=C/C(F)(F)F)NCC(Cc1ccc(C#Cc2ccc(CN3CCOCC3)cc2)cc1)c1nc[nH]c(=O)c1O] | 567.21 |
| 141 | 2-fluoro-N-(3-(4-((4- (((3aS,6aS)- hexahydropyrrolo[3,4- b]pyrrol-5(1H)-yl) methyl)phenyl)ethynyl) phenyl)-2-(5-hydroxy- 6-oxo-1,6- dihydropyrimidin-4- yl)propyl)acetamide | 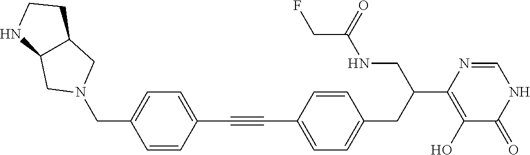 [SMILES: O=C(CF)NCC(Cc1ccc(C#Cc2ccc(CN3C[C@@H]4CCN[C@@H]4C3)cc2)cc1)c1nc[nH]c(=O)c1O] | 530.23 |
| 142 | N-(2-(5-hydroxy-6-oxo- 1,6-dihydropyrimidin- 4-yl)-3-(4-((4-((oxazol- 2-ylamino)methyl) phenyl)ethynyl)phenyl) propyl)acetamide | 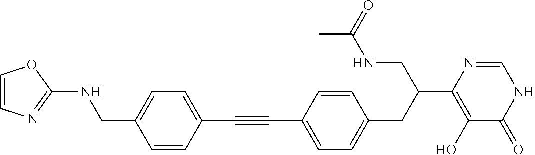 [SMILES: CC(=O)NCC(Cc1ccc(C#Cc2ccc(CNc3ncco3)cc2)cc1)c1nc[nH]c(=O)c1O] | 484.21 |
| 143 | N-(3-(4-((4- (((1R,4R,5S)-5-amino- 2-azabicyclo[2.2.1]heptan- 2- yl)methyl)phenyl)ethynyl) phenyl)-2-(5-hydroxy-6- oxo-1,6- dihydropyrimidin-4- yl)propyl)-2- fluoroacetamide | 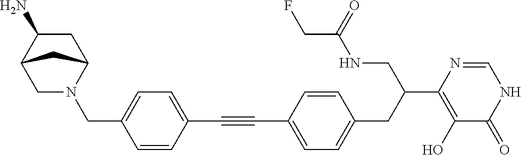 [SMILES: N[C@H]1C[C@H]2C[C@@H]1CN2Cc1ccc(C#Cc2ccc(CC(CNC(=O)CF)c3nc[nH]c(=O)c3O)cc2)cc1] | 530.25 |
| 144 | 6-(1-(allylamino)-3-(4- ((2-fluoro-4- (morpholinomethyl) phenyl)ethynyl)phenyl) propan-2-yl)-5- hydroxypyrimidin- 4(3H)-one | 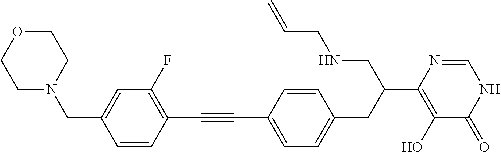 [SMILES: C=CCNCC(Cc1ccc(C#Cc2ccc(CN3CCOCC3)cc2F)cc1)c1nc[nH]c(=O)c1O] | 503.34 |
| 145 | 5-hydroxy-6-(1- (methylsulfonyl)-3-(4- ((4-(morpholinomethyl) phenyl)ethynyl)phenyl) propan-2-yl)pyrimidin- 4(3H)-one | 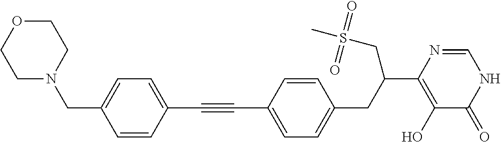 [SMILES: CS(=O)(=O)CC(Cc1ccc(C#Cc2ccc(CN3CCOCC3)cc2)cc1)c1nc[nH]c(=O)c1O] | 508.20 |
| 146 | 5-hydroxy-6-(1-hydroxy- 2-methyl-3-(4-((4- (morpholinomethyl) phenyl)ethynyl)phenyl) propan-2-yl)pyrimidin- 4(3H)-one | 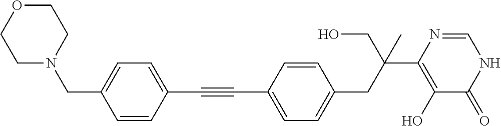 [SMILES: CC(CO)(Cc1ccc(C#Cc2ccc(CN3CCOCC3)cc2)cc1)c1nc[nH]c(=O)c1O] | 460.32 |
| 147 | 2-(4-((4-(3-(allylamino)- 2-(5-hydroxy-6-oxo- 1,6-dihydropyrimidin- 4-yl)propyl)phenyl) ethynyl)phenyl) acetonitrile | 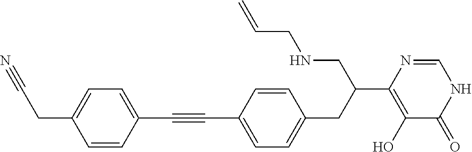 [SMILES: C=CCNCC(Cc1ccc(C#Cc2ccc(CC#N)cc2)cc1)c1nc[nH]c(=O)c1O] | 425.20 |
| 148 | 5-hydroxy-6-(1-(4-((4- (morpholinomethyl) phenyl)ethynyl)phenyl)- 3-(prop-2-yn-1- ylamino)propan-2- yl)pyrimidin-4(3H)-one | 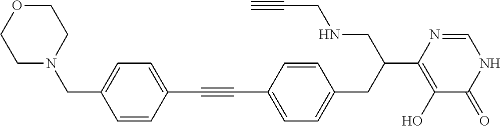 [SMILES: C#CCNCC(Cc1ccc(C#Cc2ccc(CN3CCOCC3)cc2)cc1)c1nc[nH]c(=O)c1O] | 482.24 |
| 149 | 3-(4-((4-((3- cyanoazetidin-1- yl)methyl)phenyl) ethynyl)phenyl)-N- (2,2-difluoroethyl)- 2-(5-hydroxy-6-oxo- 1,6-dihydropyrimidin- 4-yl)propanamide | 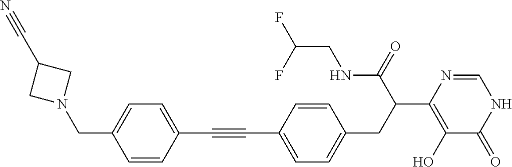 [SMILES: N#CC1CN(Cc2ccc(C#Cc3ccc(CC(C(=O)NCC(F)F)c4nc[nH]c(=O)c4O)cc3)cc2)C1] | 518.15 |
| 150 | N-(2,2-difluoroethyl)- 2-(5-hydroxy-6-oxo- 1,6-dihydropyrimidin- 4-yl)-3-(4-((4-(2- morpholinoethyl)phenyl) ethynyl)phenyl) propanamide | 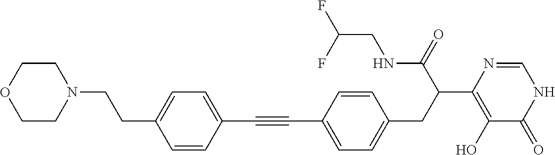 [SMILES: O=C(NCC(F)F)C(Cc1ccc(C#Cc2ccc(CCN3CCOCC3)cc2)cc1)c1nc[nH]c(=O)c1O] | 537.50 |
| 151 | N-(2,2-difluoroethyl)- 2-(5-hydroxy-6-oxo- 1,6-dihydropyrimidin- 4-yl)-3-(4-((4- (methoxymethyl)phenyl) ethynyl)phenyl) propanamide | 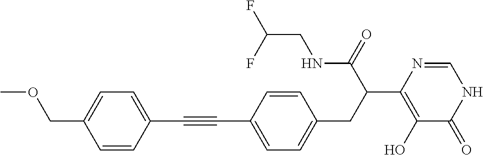 [SMILES: COCc1ccc(C#Cc2ccc(CC(C(=O)NCC(F)F)c3nc[nH]c(=O)c3O)cc2)cc1] | 468.17 |
| 152 | 3-(4-((4-((4- (cyanomethyl)piperidin- 1-yl)methyl)phenyl) ethynyl)phenyl)-N-(2,2- difluoroethyl)-2-(5- hydroxy-6-oxo-1,6- dihydropyrimidin- 4-yl)propanamide | 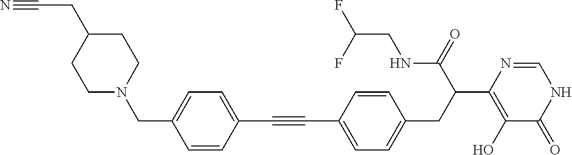 [SMILES: N#CCC1CCN(Cc2ccc(C#Cc3ccc(CC(C(=O)NCC(F)F)c4nc[nH]c(=O)c4O)cc3)cc2)CC1] | 560.14 |
| 153 | 2-(5-hydroxy-6-oxo- 1,6-dihydropyrimidin- 4-yl)-N-(2- methoxyethyl)-3-(4-((4- (morpholinomethyl) phenyl)ethynyl)phenyl) propanamide | 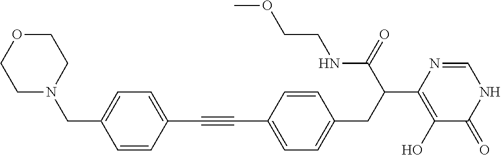 [SMILES: COCCNC(=O)C(Cc1ccc(C#Cc2ccc(CN3CCOCC3)cc2)cc1)c1nc[nH]c(=O)c1O] | 517.44 |
| 154 | N-(3,3- difluorocyclobutyl)-2- (5-hydroxy-6-oxo-1,6- dihydropyrimidin-4-yl)- 3-(4-((4- (morpholinomethyl) phenyl)ethynyl)phenyl) propanamide | 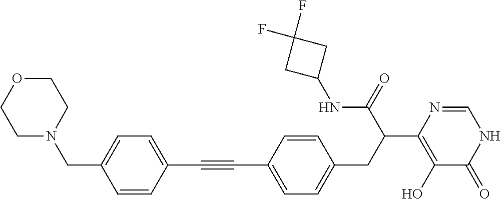 [SMILES: O=C(NC1CC(F)(F)C1)C(Cc1ccc(C#Cc2ccc(CN3CCOCC3)cc2)cc1)c1nc[nH]c(=O)c1O] | 549.32 |
| 155 | 3-(4-((4-(cyanomethyl) phenyl)ethynyl)phenyl)- N-(2,2-difluoroethyl)-2- (5-hydroxy-6-oxo-1,6- dihydropyrimidin- 4-yl)propanamide | 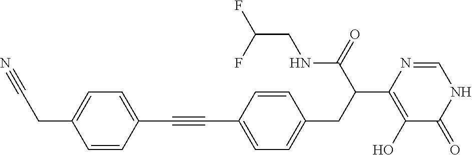 [SMILES: N#CCc1ccc(C#Cc2ccc(CC(C(=O)NCC(F)F)c3nc[nH]c(=O)c3O)cc2)cc1] | 463.15 |
| 156 | 3-(4-((4-((3- (cyanomethyl) azetidin-1-yl)methyl) phenyl)ethynyl)phenyl)- N-(2,2-difluoroethyl)-2- (5-hydroxy-6-oxo-1,6- dihydropyrimidin-4- yl)propanamide | 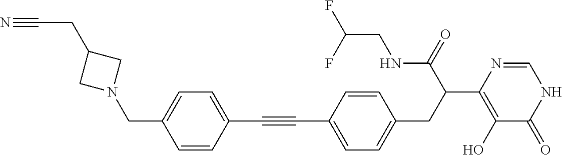 [SMILES: N#CCC1CN(Cc2ccc(C#Cc3ccc(CC(C(=O)NCC(F)F)c4nc[nH]c(=O)c4O)cc3)cc2)C1] | 532.21 |
| 157 | 3-(4-((4-(((S)-3- cyanopyrrolidin-1- yl)methyl)phenyl) ethynyl)phenyl)-N- (2,2-difluoroethyl)- 2-(5-hydroxy-6-oxo- 1,6-dihydropyrimidin- 4-yl)propanamide | 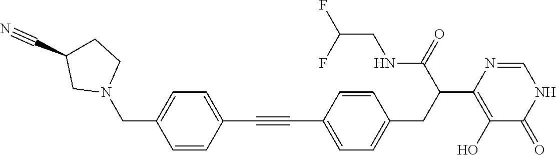 [SMILES: N#C[C@H]1CCN(Cc2ccc(C#Cc3ccc(CC(C(=O)NCC(F)F)c4nc[nH]c(=O)c4O)cc3)cc2)C1] | 532.11 |
| 158 | 3-(4-((4-(((2-(1H- imidazol-1-yl)ethyl) amino)methyl)phenyl) ethynyl)phenyl)-N- (2,2-difluoroethyl)-2- (5-hydroxy-6-oxo-1,6- dihydropyrimidin-4- yl)propanamide | 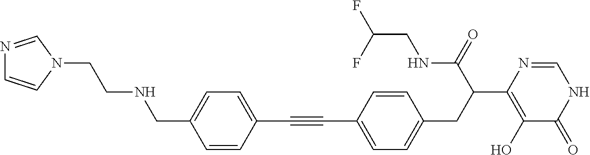 [SMILES: O=C(NCC(F)F)C(Cc1ccc(C#Cc2ccc(CNCCn3ccnc3)cc2)cc1)c1nc[nH]c(=O)c1O] | 547.12 |
| 159 | 3-(4-((4-cyanophenyl) ethynyl)phenyl)-N-(2,2- difluoroethyl)-2-(5- hydroxy-6-oxo-1,6- dihydropyrimidin-4- yl)propanamide | 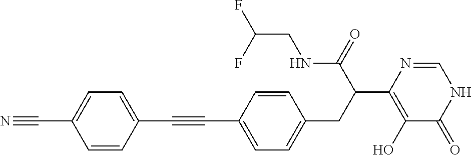 [SMILES: N#Cc1ccc(C#Cc2ccc(CC(C(=O)NCC(F)F)c3nc[nH]c(=O)c3O)cc2)cc1] | 449.23 |
| 160 | 3-(4-((4-(chloromethyl) phenyl)ethynyl)phenyl)- N-(2,2-difluoroethyl)-2- (5-hydroxy-6-oxo-1,6- dihydropyrimidin-4- yl)propanamide | 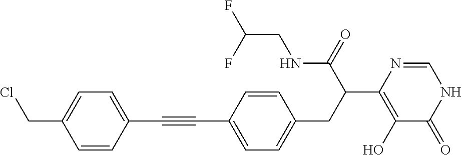 [SMILES: O=C(NCC(F)F)C(Cc1ccc(C#Cc2ccc(CCl)cc2)cc1)c1nc[nH]c(=O)c1O] | 473.11 |
| 161 | N-(2,2-difluoroethyl)-2- (5-hydroxy-6-oxo-1,6- dihydropyrimidin-4-yl)- 3-(4-((4-propylphenyl) ethynyl)phenyl) propanamide | 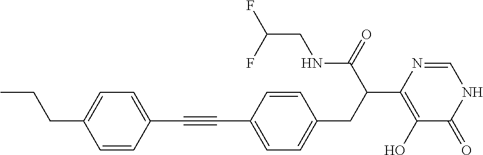 [SMILES: CCCc1ccc(C#Cc2ccc(CC(C(=O)NCC(F)F)c3nc[nH]c(=O)c3O)cc2)cc1] | 466.19 |
| 162 | N-(1-cyanoethyl)-2-(5- hydroxy-6-oxo-1,6- dihydropyrimidin-4-yl)- 3-(4-((4- (morpholinomethyl) phenyl)ethynyl)phenyl) propanamide | 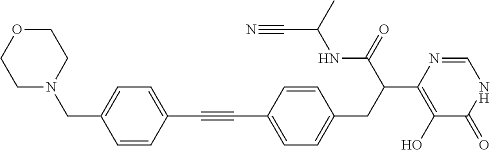 [SMILES: CC(C#N)NC(=O)C(Cc1ccc(C#Cc2ccc(CN3CCOCC3)cc2)cc1)c1nc[nH]c(=O)c1O] | 511.58 |
| 163 | N-(2,2-difluoroethyl)-2- (5-hydroxy-6-oxo-1,6- dihydropyrimidin-4-yl)- 3-(4-((4-((4- hydroxypiperidin-1- yl)methyl)phenyl) ethynyl)phenyl) propanamide | 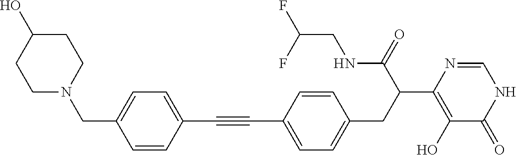 [SMILES: O=C(NCC(F)F)C(Cc1ccc(C#Cc2ccc(CN3CCC(O)CC3)cc2)cc1)c1nc[nH]c(=O)c1O] | 537.38 |
| 164 | 3-(4-((4-((3,3- difluoroazetidin-1- yl)methyl)phenyl) ethynyl)phenyl)-N- (2,2-difluoroethyl)- 2-(5-hydroxy-6-oxo- 1,6-dihydropyrimidin- 4-yl)propanamide | 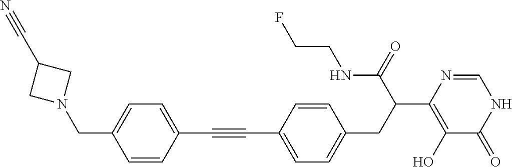 [SMILES: N#CC1CN(Cc2ccc(C#Cc3ccc(CC(C(=O)NCCF)c4nc[nH]c(=O)c4O)cc3)cc2)C1] | 529.31 |
| 165 | 3-(4-((4-((3- cyanoazetidin-1- yl)methyl)phenyl) ethynyl)phenyl)-N- (2-fluoroethyl)-2-(5- hydroxy-6-oxo-1,6- dihydropyrimidin-4- yl)propanamide | 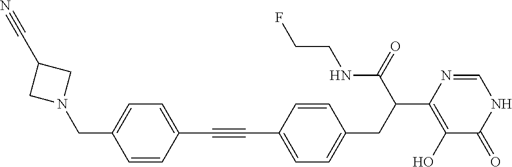 [SMILES: N#CC1CN(Cc2ccc(C#Cc3ccc(CC(C(=O)NCCF)c4nc[nH]c(=O)c4O)cc3)cc2)C1] | 500.15 |
| 166 | N-(2,2-difluoroethyl)- 2-(5-hydroxy-6-oxo- 1,6-dihydropyrimidin- 4-yl)-3-(4- (phenylethynyl)phenyl) propanamide | 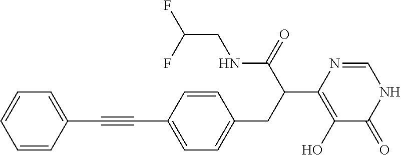 [SMILES: O=C(NCC(F)F)C(Cc1ccc(C#Cc2ccccc2)cc1)c1nc[nH]c(=O)c1O] | 424.24 |
| 167 | ethyl 3-(2-fluoro-4-((4- (morpholinomethyl) phenyl)ethynyl)phenyl)- 2-(5-hydroxy-6-oxo-1,6- dihydropyrimidin-4- yl)propanoate | 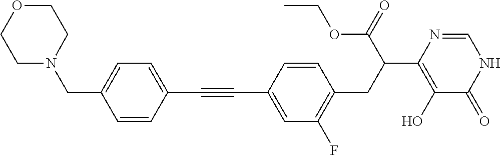 [SMILES: CCOC(=O)C(Cc1ccc(C#Cc2ccc(CN3CCOCC3)cc2)cc1F)c1nc[nH]c(=O)c1O] | 506.15 |
| 168 | N-(2,2-difluoroethyl)-2- (5-hydroxy-6-oxo-1,6- dihydropyrimidin-4-yl)- 3-(4-((4-(((3- hydroxycyclobutyl) amino)methyl)phenyl) ethynyl)phenyl) propanamide | 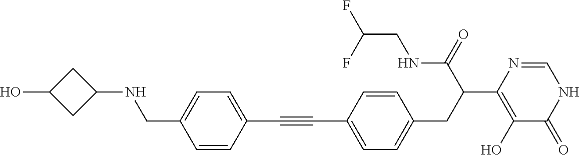 [SMILES: O=C(NCC(F)F)C(Cc1ccc(C#Cc2ccc(CNC3CC(O)C3)cc2)cc1)c1nc[nH]c(=O)c1O] | 523.12 |
| 169 | N-(2,2-difluoroethyl)- 2-(5-hydroxy-6-oxo- 1,6-dihydropyrimidin- 4-yl)-3-(4-((4-((6- (hydroxymethyl)-3- azabicyclo[3.1.0]hexan- 3-yl)methyl)phenyl) ethynyl)phenyl) propanamide | 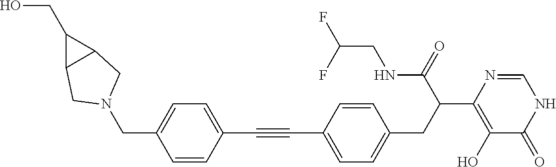 [SMILES: O=C(NCC(F)F)C(Cc1ccc(C#Cc2ccc(CN3CC4C(CO)C4C3)cc2)cc1)c1nc[nH]c(=O)c1O] | 549.19 |
| 170 | N-(2,2-difluoroethyl)-2- (5-hydroxy-6-oxo-1,6- dihydropyrimidin-4-yl)- 3-(4-((4-(((2- hydroxyethyl)amino) methyl)phenyl)ethynyl) phenyl)propanamide | 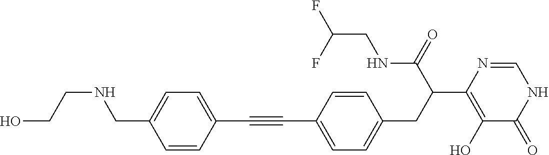 [SMILES: O=C(NCC(F)F)C(Cc1ccc(C#Cc2ccc(CNCCO)cc2)cc1)c1nc[nH]c(=O)c1O] | 497.31 |
| 171 | N-(2,2-difluoroethyl)-2- (5-hydroxy-6-oxo-1,6- dihydropyrimidin-4-yl)- 3-(4-((4-(((2- (methylsulfonyl)ethyl) amino)methyl)phenyl) ethynyl)phenyl) propanamide | 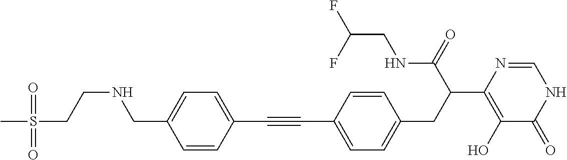 [SMILES: CS(=O)(=O)CCNCc1ccc(C#Cc2ccc(CC(C(=O)NCC(F)F)c3nc[nH]c(=O)c3O)cc2)cc1] | 559.19 |
| 172 | N-(2,2-difluoroethyl)-2- (5-hydroxy-6-oxo-1,6- dihydropyrimidin-4-yl)- 3-(4-((4-((3- hydroxyazetidin-1- yl)methyl)phenyl) ethynyl)phenyl) propanamide | 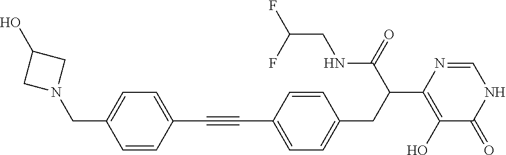 [SMILES: O=C(NCC(F)F)C(Cc1ccc(C#Cc2ccc(CN3CC(O)C3)cc2)cc1)c1nc[nH]c(=O)c1O] | 509.21 |
| 173 | 3-(4-((4- (acetamidomethyl) phenyl)ethynyl)phenyl)- N-(2,2-difluoroethyl)-2- (5-hydroxy-6-oxo-1,6- dihydropyrimidin-4- yl)propanamide | 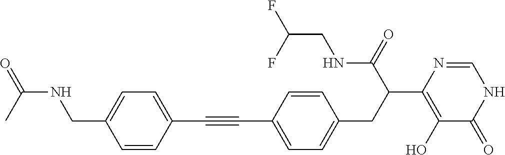 [SMILES: CC(=O)NCc1ccc(C#Cc2ccc(CC(C(=O)NCC(F)F)c3nc[nH]c(=O)c3O)cc2)cc1] | 495.20 |
| 174 | 3-(4-((4-(((cyanomethyl) amino)methyl)phenyl) ethynyl)phenyl)- N-(2,2-difluoroethyl)- 2-(5-hydroxy-6-oxo- 1,6-dihydropyrimidin- 4-yl)propanamide | 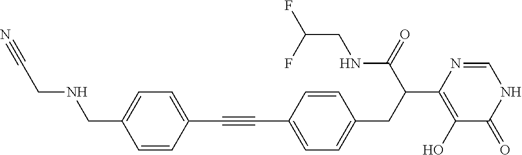 [SMILES: N#CCNCc1ccc(C#Cc2ccc(CC(C(=O)NCC(F)F)c3nc[nH]c(=O)c3O)cc2)cc1] | 492.32 |
| 175 | N-(2,2-difluoromethyl)- 2-(5-hydroxy-6-oxo-1,6- dihydropyrimidin-4-yl)- 3-(4-((4- (methylsulfonyl) phenyl)ethynyl)phenyl) propanamide | 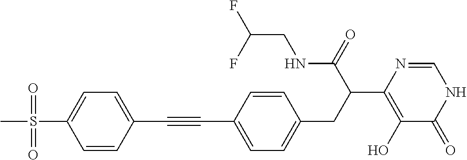 [SMILES: CS(=O)(=O)c1ccc(C#Cc2ccc(CC(C(=O)NCC(F)F)c3nc[nH]c(=O)c3O)cc2)cc1] | 502.22 |
| 176 | N-(2,2-difluoroethyl)-3- (4-((4-(((2R,6S)-2,6- dimethylmorpholino) methyl)phenyl)ethynyl) phenyl)-2-(5-hydroxy-6- oxo-1,6- dihydropyrimidin-4- yl)propanamide | 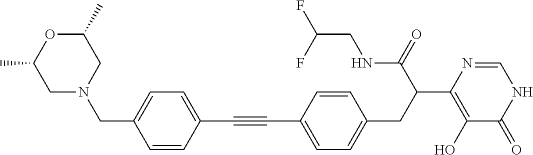 [SMILES: C[C@@H]1CN(Cc2ccc(C#Cc3ccc(CC(C(=O)NCC(F)F)c4nc[nH]c(=O)c4O)cc3)cc2)C[C@H](C)O1] | 551.21 |
| 177 | N-(2,2-difluoroethyl)-2- (5-hydroxy-6-oxo-1,6- dihydropyrimidin-4-yl)- 3-(4-((4-(((R)-2- (hydroxymethyl) pyrrolidin-1-yl)methyl) phenyl)ethynyl)phenyl) propanamide | 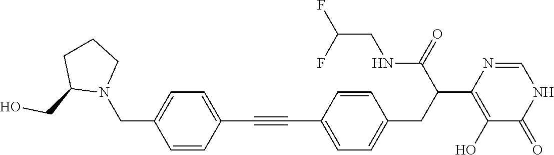 [SMILES: O=C(NCC(F)F)C(Cc1ccc(C#Cc2ccc(CN3CCC[C@@H]3CO)cc2)cc1)c1nc[nH]c(=O)c1O] | 537.33 |
| 178 | 3-(4-((3-(cyanomethyl) phenyl)ethynyl)phenyl)- N-(2,2-difluoroethyl)-2- (5-hydroxy-6-oxo-1,6- dihydropyrimidin-4- yl)propanamide | 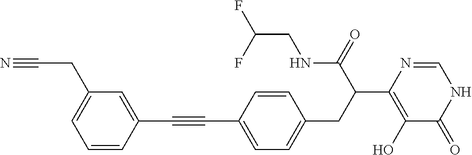 [SMILES: N#CCc1cccc(C#Cc2ccc(CC(C(=O)NCC(F)F)c3nc[nH]c(=O)c3O)cc2)c1] | 463.16 |
| 179 | N-(2-cyanoethyl)-2-(5- hydroxy-6-oxo-1,6- dihydropyrimidin-4-yl)- 3-(4-((4- (morpholinomethyl) phenyl)ethynyl)phenyl) propanamide | 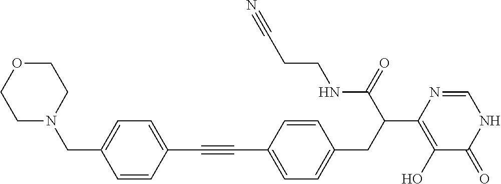 [SMILES: N#CCCNC(=O)C(Cc1ccc(C#Cc2ccc(CN3CCOCC3)cc2)cc1)c1nc[nH]c(=O)c1O] | 512.28 |
| 180 | N-(2,2-difluoroethyl)-3- (4-((4-(((3aS,6aS)- hexahydropyrrolo[3,4- b]pyrrol-5(1H)- yl)methyl)phenyl) ethynyl)phenyl)-2- (5-hydroxy-6-oxo-1,6- dihydropyrimidin-4- yl)propanamide | 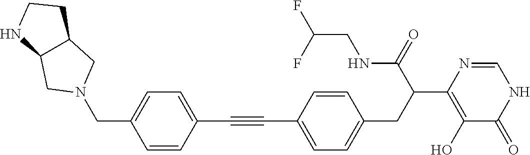 [SMILES: O=C(NCC(F)F)C(Cc1ccc(C#Cc2ccc(CN3C[C@@H]4CCN[C@@H]4C3)cc2)cc1)c1nc[nH]c(=O)c1O] | 548.34 |
| 181 | N-(2,2-difluoroethyl)-2- (5-hydroxy-6-oxo-1,6- dihydropyrimidin-4-yl)- 3-(4-((3- (hydroxymethyl)phenyl) ethynyl)phenyl) propanamide | 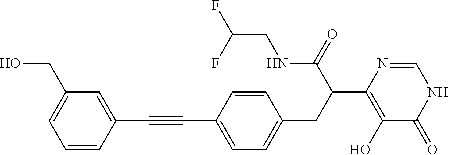 [SMILES: O=C(NCC(F)F)C(Cc1ccc(C#Cc2cccc(CO)c2)cc1)c1nc[nH]c(=O)c1O] | 454.15 |
| 182 | 3-(4-((4-((3- cyanoazetidin-1- yl)methyl)phenyl) ethynyl)phenyl)-N-(2- cyanoethyl)-2-(5- hydroxy-6-oxo-1,6- dihydropyrimidin-4- yl)propanamide | 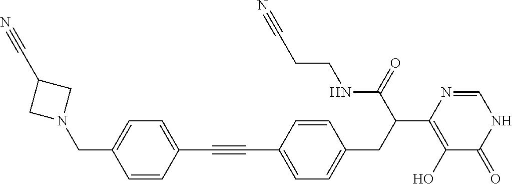 [SMILES: N#CCCNC(=O)C(Cc1ccc(C#Cc2ccc(CN3CC(C#N)C3)cc2)cc1)c1nc[nH]c(=O)c1O] | 507.31 |
| 183 | 3-(4-((4-(2-cyanoethyl) phenyl)ethynyl)phenyl)- N-(2,2-difluoroethyl)-2- (5-hydroxy-6-oxo-1,6- dihydropyrimidin-4- yl)propanamide | 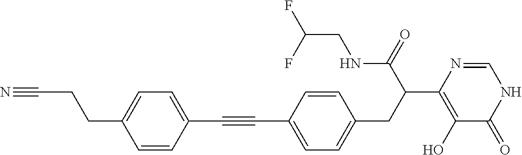 [SMILES: N#CCCc1ccc(C#Cc2ccc(CC(C(=O)NCC(F)F)c3nc[nH]c(=O)c3O)cc2)cc1] | 477.18 |
| 184 | N-(cyanomethyl)-2- (5-hydroxy-6-oxo-1,6- dihydropyrimidin-4-yl)- 3-(4-((4-((((R)- tetrahydrofuran-3- yl)amino)methyl) phenyl)ethynyl) propanamide | 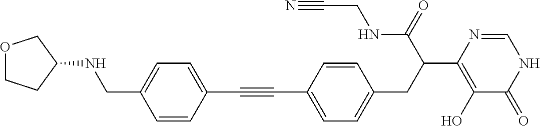 [SMILES: N#CCNC(=O)C(Cc1ccc(C#Cc2ccc(CN[C@@H]3CCOC3)cc2)cc1)c1nc[nH]c(=O)c1O] | 498.25 |
| 185 | 6-(4-((2-fluoro-4- (morpholinomethyl) phenyl)ethynyl) phenethyl)-5- hydroxypyrimidin- 4(3H)-one | 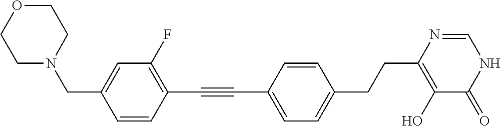 [SMILES: O=c1[nH]cnc(CCc2ccc(C#Cc3ccc(CN4CCOCC4)cc3F)cc2)c1O] | 434.33 |
| 186 | 6-(4-((3-fluoro-4- (morpholinomethyl) phenyl)ethynyl) phenethyl)-5- hydroxypyrimidin- 4(3H)-one | 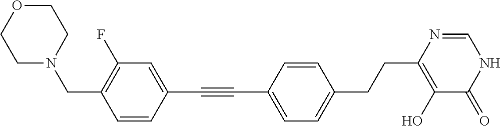 [SMILES: O=c1[nH]cnc(CCc2ccc(C#Cc3ccc(CN4CCOCC4)c(F)c3)cc2)c1O] | 434.24 |
| 187 | 6-(3-fluoro-4-((4-((3- hydroxyazetidin-1- yl)methyl)phenyl) ethynyl)phenethyl)-5- hydroxypyrimidin- 4(3H)-one | 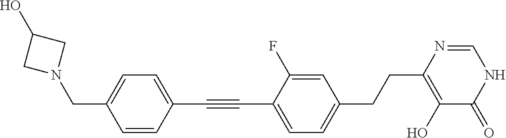 [SMILES: O=c1[nH]cnc(CCc2ccc(C#Cc3ccc(CN4CC(O)C4)cc3)c(F)c2)c1O] | 420.36 |
| 188 | 1-(4-((2-fluoro-4-(2- (5-hydroxy-6-oxo-1,6- dihydropyrimidin-4- yl)ethyl)phenyl)ethynyl) benzyl)azetidine-3- carbonitrile | 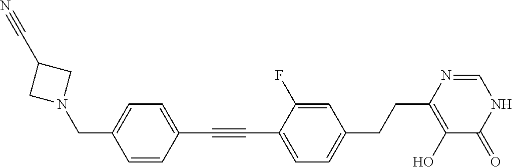 [SMILES: N#CC1CN(Cc2ccc(C#Cc3ccc(CCc4nc[nH]c(=O)c4O)cc3F)cc2)C1] | 429.27 |
| 189 | 6-(3-fluoro-4-((4-((3- hydroxyazetidin-1- yl)methyl)phenyl) ethynyl)phenethyl)-5- hydroxypyrimidin- 4(3H)-one | 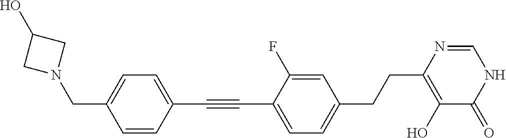 [SMILES: O=c1[nH]cnc(CCc2ccc(C#Cc3ccc(CN4CC(O)C4)cc3)c(F)c2)c1O] | 420.26 |
| 190 | 6-(3-fluoro-4-((4-((2- methylmorpholino) methyl)phenyl)ethynyl) phenethyl)-5- hydroxypyrimidin- 4(3H)-one | 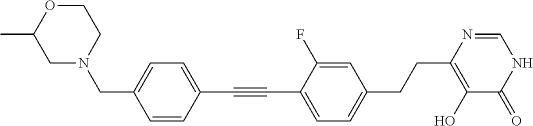 [SMILES: CC1CN(Cc2ccc(C#Cc3ccc(CCc4nc[nH]c(=O)c4O)cc3F)cc2)CCO1] | 448.41 |
| 191 | 6-(3-fluoro-4- (phenylethynyl) phenethyl)-5- hydroxypyrimidin- 4(3H)-one | 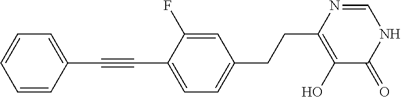 [SMILES: O=c1[nH]cnc(CCc2ccc(C#Cc3ccccc3)c(F)c2)c1O] | 335.34 |
| 192 | 6-(4-((4-((3- chloroazetidin-1- yl)methyl)phenyl) ethynyl)-3- fluorophenethyl)-5- hydroxypyrimidin- 4(3H)-one | 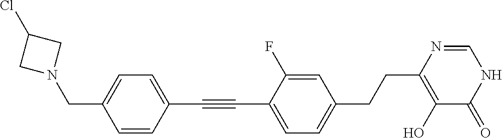 [SMILES: O=c1[nH]cnc(CCc2ccc(C#Cc3ccc(CN4CC(Cl)C4)cc3)c(F)c2)c1O] | 439.43 |
| 193 | 6-(4-((4-((bis(2- methoxyethyl) amino)methyl)phenyl) ethynyl)-3- fluorophenethyl)-5- hydroxypyrimidin- 4(3H)-one | 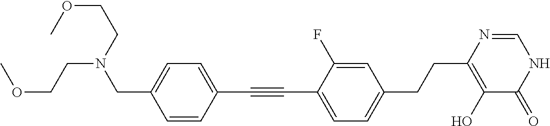 [SMILES: COCCN(CCOC)Cc1ccc(C#Cc2ccc(CCc3nc[nH]c(=O)c3O)cc2F)cc1] | 480.25 |
| 194 | 6-(1-(3-fluoro-4-((4- (morpholinomethyl) phenyl)ethynyl) phenyl)-3- hydroxypropan-2-yl)- 5-hydroxypyrimidin- 4(3H)-one | 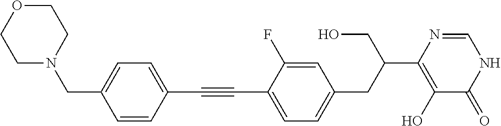 [SMILES: O=c1[nH]cnc(C(CO)Cc2ccc(C#Cc3ccc(CN4CCOCC4)cc3)c(F)c2)c1O] | 464.35 |
| 195 | 6-(1-((2-fluoroethyl) amino)-3-(4-((4- (hydroxymethyl) phenyl)ethynyl) phenyl)propan-2-yl)- 5-hydroxypyrimidin- 4(3H)-one | 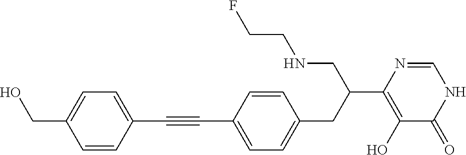 [SMILES: O=c1[nH]cnc(C(CNCCF)Cc2ccc(C#Cc3ccc(CO)cc3)cc2)c1O] | 422.28 |
| 196 | 6-(1-((2-fluoroethyl) amino)-3-(4-((4-((3- hydroxyazetidin-1- yl)methyl)phenyl) ethynyl)phenyl) propan-2-yl)-5- hydroxypyrimidin- 4(3H)-one | 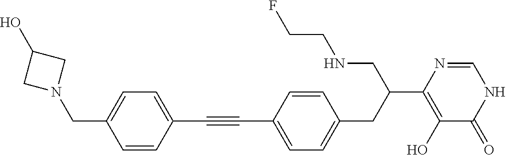 [SMILES: O=c1[nH]cnc(C(CNCCF)Cc2ccc(C#Cc3ccc(CN4CC(O)C4)cc3)cc2)c1O] | 477.25 |
| 197 | 5-hydroxy-6-(1-(4- ((4-(morpholinomethyl) phenyl)ethynyl)phenyl)- 3-(oxetan-3- ylamino)propan-2- yl)pyrimidin-4(3H)-one | 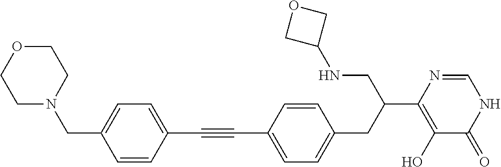 [SMILES: O=c1[nH]cnc(C(CNC2COC2)Cc2ccc(C#Cc3ccc(CN4CCOCC4)cc3)cc2)c1O] | 501.10 |
| 198 | 6-(1-((2-fluoroethyl) amino)-3-(4-((4- (((tetrahydrofuran-3- yl)amino)methyl) phenyl)ethynyl)phenyl) propan-2-yl)-5- hydroxypyrimidin- 4(3H)-one | 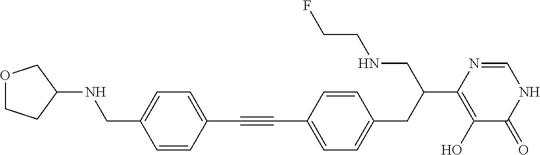 [SMILES: O=c1[nH]cnc(C(CNCCF)Cc2ccc(C#Cc3ccc(CNC4CCOC4)cc3)cc2)c1O] | 491.28 |
| 200 | N-(3-(4-((4-((2- ((dimethylamino) methyl)morpholino) methyl)phenyl) ethynyl)phenyl)-2- (5-hydroxy-6-oxo- 1,6-dihydropyrimidin- 4-yl)propyl)-2- fluoroacetamide | 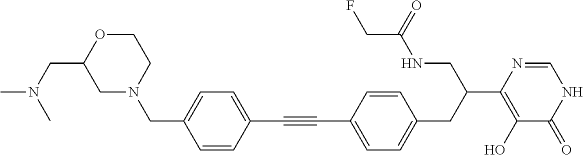 [SMILES: CN(C)CC1CN(Cc2ccc(C#Cc3ccc(CC(CNC(=O)CF)c4nc[nH]c(=O)c4O)cc3)cc2)CCO1] | 562.41 |
| 201 | 2-fluoro-N-(2-(5- hydroxy-6-oxo-1,6- dihydropyrimidin-4- yl)-3-(4-((4-((1- oxidoethiomorpholino) methyl)phenyl)ethynyl) phenyl)propyl)acetamide | 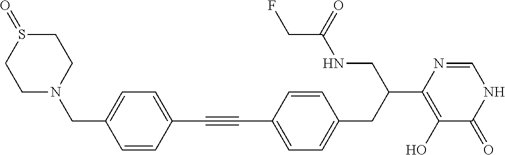 [SMILES: O=C(CF)NCC(Cc1ccc(C#Cc2ccc(CN3CCS(=O)CC3)cc2)cc1)c1nc[nH]c(=O)c1O] | 537.32 |
| 202 | 2-fluoro-N-(2-(5- hydroxy-6-oxo-1,6- dihydropyrimidin-4- yl)-3-(4-((4-(((S)-3- methoxypyrrolidin-1- yl)methyl)phenyl) ethynyl)phenyl)propyl) acetamide | 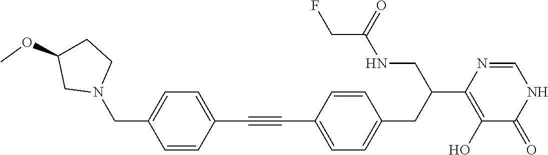 [SMILES: CO[C@H]1CCN(Cc2ccc(C#Cc3ccc(CC(CNC(=O)CF)c4nc[nH]c(=O)c4O)cc3)cc2)C1] | 519.13 |
| 203 | 2-fluoro-N-(2-(5- hydroxy-6-oxo-1,6- dihydropyrimidin-4- yl)-3-(4-((4-(((R)-3- methoxypyrrolidin-1- yl)methyl)phenyl) ethynyl)phenyl)propyl) acetamide | 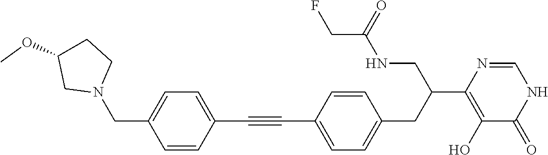 [SMILES: CO[C@@H]1CCN(Cc2ccc(C#Cc3ccc(CC(CNC(=O)CF)c4nc[nH]c(=O)c4O)cc3)cc2)C1] | 519.24 |
| 204 | 6-(1-((3,3- difluorocyclobutyl) amino)-3-(4-((4- (morpholinomethyl) phenyl)ethynyl)phenyl) propan-2-yl)-5- hydroxypyrimidin- 4(3H)-one | 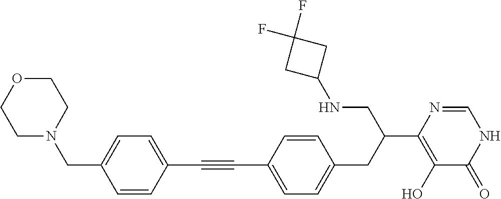 [SMILES: O=c1[nH]cnc(C(CNC2CC(F)(F)C2)Cc2ccc(C#Cc3ccc(CN4CCOCC4)cc3)cc2)c1O] | 535.31 |
| 205 | 6-(1-((2-fluoroethyl) amino)-3-(4-((4-((1- oxidothiomorpholino) methyl)phenyl)ethynyl) phenyl)propan-2-yl)-5- hydroxypyrimidin- 4(3H)-one | 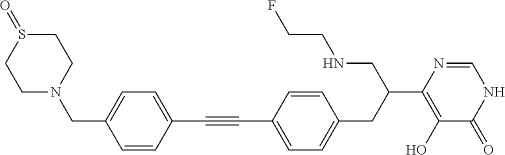 [SMILES: O=c1[nH]cnc(C(CNCCF)Cc2ccc(C#Cc3ccc(CN4CCS(=O)CC4)cc3)cc2)c1O] | 523.41 |
| 206 | N-(4-((4-(3-((2- fluoroethyl)amino)-2- (5-hydroxy-6-oxo- 1,6-dihydropyrimidin- 4-yl)propyl)phenyl) ethynyl)benzyl) acetamide | 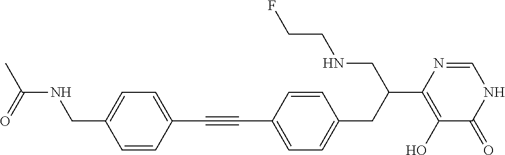 [SMILES: CC(=O)NCc1ccc(C#Cc2ccc(CC(CNCCF)c3nc[nH]c(=O)c3O)cc2)cc1] | 463.12 |
| 207 | 6-(1-((2-fluoroethyl) amino)-3-(4-((4- ((oxetan-3-ylamino) methyl)phenyl)ethynyl) phenyl)propan-2-yl)-5- hydroxypyrimidin- 4(3H)-one | 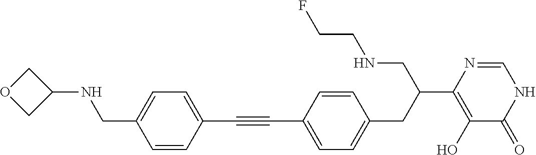 [SMILES: O=c1[nH]cnc(C(CNCCF)Cc2ccc(C#Cc3ccc(CNC4COC4)cc3)cc2)c1O] | 477.50 |
| 209 | 1-(4-((4-(3-((2- fluoroethyl)amino)-2- (5-hydroxy-6-oxo- 1,6-dihydropyrimidin- 4-yl)propyl)phenyl) ethynyl)benzyl) azetidine-3-carbonitrile | 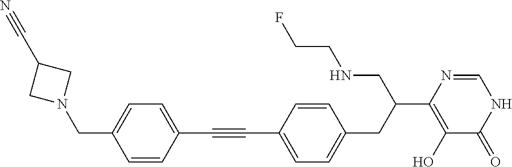 [SMILES: N#CC1CN(Cc2ccc(C#Cc3ccc(CC(CNCCF)c4nc[nH]c(=O)c4O)cc3)cc2)C1] | 486.33 |
| 210 | N-(2-(5-hydroxy-2- methyl-6-oxo-1,6- dihydropyrimidin-4- yl)-3-(4-((4- (morpholinomethyl) phenyl)ethynyl)phenyl) propyl)acetamide | 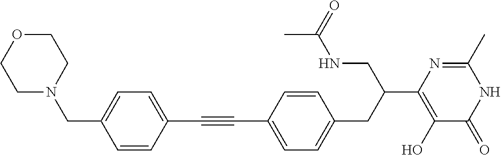 [SMILES: CC(=O)NCC(Cc1ccc(C#Cc2ccc(CN3CCOCC3)cc2)cc1)c1nc(C)[nH]c(=O)c1O] | 501.23 |
| 214 | 6-(1-((3- fluorocyclobutyl) amino)-3-(4-((4- (morpholinomethyl) phenyl)ethynyl)phenyl) propan-2-yl)-5- hydroxypyrimidin- 4(3H)-one | 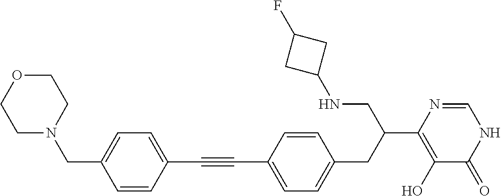 [SMILES: O=c1[nH]cnc(C(CNC2CC(F)C2)Cc2ccc(C#Cc3ccc(CN4CCOCC4)cc3)cc2)c1O] | 517.16 |
| 215 | 6-(1-(4-((4-((8-oxa-3- azabicyclo[3.2.1]octan- 3-yl)methyl)phenyl) ethynyl)phenyl)-3-((2- fluoroethyl)amino) propan-2-yl)-5- hydroxypyrimidin- 4(3H)-one | 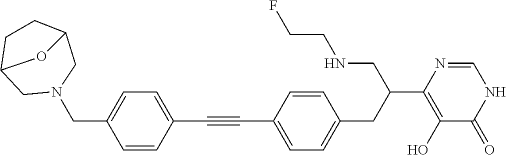 [SMILES: O=c1[nH]cnc(C(CNCCF)Cc2ccc(C#Cc3ccc(CN4CC5CCC(C4)O5)cc3)cc2)c1O] | 517.62 |
| 216 | 6-(1-(3-fluoro-4-((4- (morpholinomethyl) phenyl)ethynyl)phenyl)- 3-((2-fluoroethyl)amino) propan-2-yl)-5- hydroxypyrimidin- 4(3H)-one | 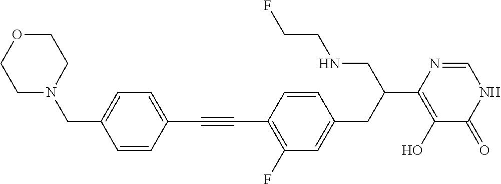 [SMILES: O=c1[nH]cnc(C(CNCCF)Cc2ccc(C#Cc3ccc(CN4CCOCC4)cc3)c(F)c2)c1O] | 509.37 |
| 217 | methyl 2-(4-((4-(3-((2- fluoroethyl)amino)-2- (5-hydroxy-6-oxo-1,6- dihydropyrimidin-4- yl)propyl)phenyl) ethynyl)phenyl)acetate | 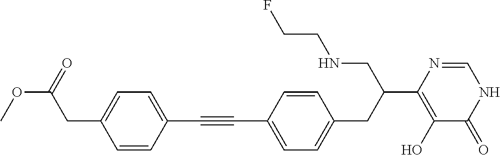 [SMILES: COC(=O)Cc1ccc(C#Cc2ccc(CC(CNCCF)c3nc[nH]c(=O)c3O)cc2)cc1] | 464.18 |
| 218 | 6-(1-(4-((4-(((2R,6R)- 2,6-dimethylmorpholino) methyl)phenyl)ethynyl) phenyl)-3-((2-fluoroethyl) amino)propan-2-yl)-5- hydroxypyrimidin-4(3H)- one | 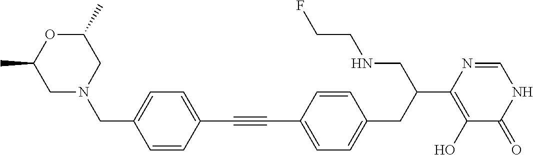 [SMILES: C[C@@H]1CN(Cc2ccc(C#Cc3ccc(CC(CNCCF)c4nc[nH]c(=O)c4O)cc3)cc2)C[C@@H](C)O1] | 519.43 |
| 219 | N-(2,2-difluoroethyl)- 2-(5-hydroxy-6-oxo- 1,6-dihydropyrimidin- 4-yl)-3-(4-((4-((4- (hydroxymethyl) piperidin-1- yl)methyl)phenyl) ethynyl)phenyl) propanamide | 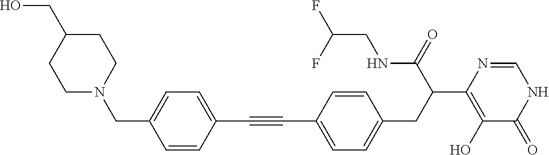 [SMILES: O=C(NCC(F)F)C(Cc1ccc(C#Cc2ccc(CN3CCC(CO)CC3)cc2)cc1)c1nc[nH]c(=O)c1O] | 551.26 |
| 246 | 5-hydroxy-6-(1-(4- ((4-(morpholinomethyl) phenyl)ethynyl)benzyl) cyclopropyl)pyrimidin- 4(3H)-one | 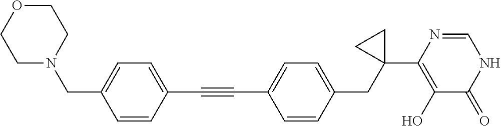 [SMILES: O=c1[nH]cnc(C2(Cc3ccc(C#Cc4ccc(CN5CCOCC5)cc4)cc3)CC2)c1O] | 442.30 |
| 247 | (R)-5-hydroxy-6-(1-(4- ((4-(((tetrahydrofuran- 3-yl)amino)methyl) phenyl)ethynyl)benzyl) cyclopropyl)pyrimidin- 4(3H)-one | 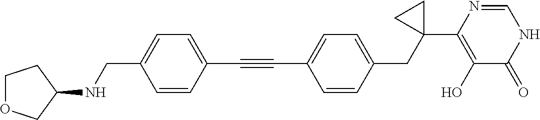 [SMILES: O=c1[nH]cnc(C2(Cc3ccc(C#Cc4ccc(CN[C@@H]5CCOC5)cc4)cc3)CC2)c1O] | 442.43 |
| 248 | 6-(4-((3-chlorophenyl) ethynyl)phenethyl)-5- hydroxypyrimidin- 4(3H)-one | 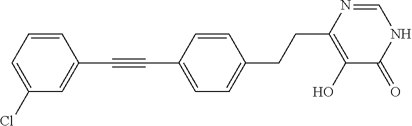 [SMILES: O=c1[nH]cnc(CCc2ccc(C#Cc3cccc(Cl)c3)cc2)c1O] | 351.9 |
| 249 | 5-hydroxy-6-(1- morpholino-3-(4-((4- (morpholinomethyl) phenyl)ethynyl)phenyl) propan-2-yl)pyrimidin- 4(3H)-one | 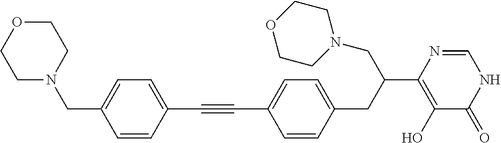 [SMILES: O=c1[nH]cnc(C(Cc2ccc(C#Cc3ccc(CN4CCOCC4)cc3)cc2)CN2CCOCC2)c1O] | 515.3 |
| 250 | 5-hydroxy-6-(1-(4-((4- (morpholinomethyl) phenyl)ethynyl)phenyl)- 3-((pyridin-3-ylmethyl) amino)propan-2- yl)pyrimidin-4(3H)-one | 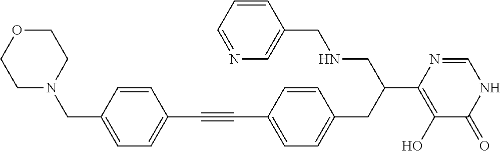 [SMILES: O=c1[nH]cnc(C(CNCc2cccnc2)Cc2ccc(C#Cc3ccc(CN4CCOCC4)cc3)cc2)c1O] | 536.45 |
| 251 | 5-hydroxy-6-(1-(4-((4- (morpholinomethyl) phenyl)ethynyl)phenyl)- 3-((2-(pyridin-3- yl)ethyl)amino)propan- 2-yl)pyrimidin-4(3H)- one | 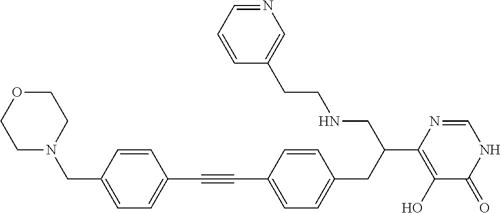 [SMILES: O=c1[nH]cnc(C(CNCCc2cccnc2)Cc2ccc(C#Cc3ccc(CN4CCOCC4)cc3)cc2)c1O] | 550.15 |
| 252 | 6-(1-((2-fluoroethyl) amino)-3-(4-((4-(2- morpholinoethyl)phenyl) ethynyl)phenyl)propan- 2-yl)-5- hydroxypyrimidin- 4(3H)-one | 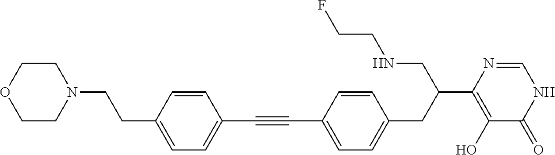 [SMILES: O=c1[nH]cnc(C(CNCCF)Cc2ccc(C#Cc3ccc(CCN4CCOCC4)cc3)cc2)c1O] | |
| 253 | 6-(1-(ethylamino)-3-(4- ((4-(morpholinomethyl) phenyl)ethynyl)phenyl) propan-2-yl)-5- hydroxypyrimidin- 4(3H)-one | 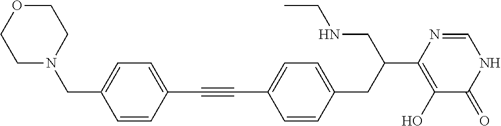 [SMILES: CCNCC(Cc1ccc(C#Cc2ccc(CN3CCOCC3)cc2)cc1)c1nc[nH]c(=O)c1O] | 473.3 |

In some embodiments, the heterocyclic LpxC inhibitory compound described in Formula (I), (Ia), (IIa) or (IIIa) has a structure provided in Table 2.

TABLE 2

| Compound | | |
|---|---|---|
| No. | Name | Structure |
| 220 | 5-hydroxy-6-(1- (((methylsulfonyl)methyl)amino)-3- (4-((4- (morpholinomethyl)phenyl)ethynyl) phenyl)propan-2-yl)pyrimidin-4(3H)- one | 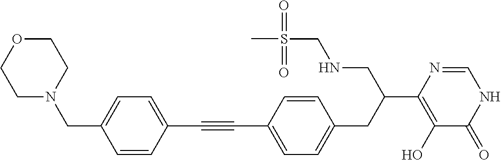 [SMILES: CS(=O)(=O)CNCC(Cc1ccc(C#Cc2ccc(CN3CCOCC3)cc2)cc1)c1nc[nH]c(=O)c1O] |
| 221 | 6-(1-(1H-imidazol-2-y1)-2-(4-((4- (morpholinomethyl)phenyl)ethynyl) phenyl)ethyl)-5-hydroxypyrimidin- 4(3H)-one | 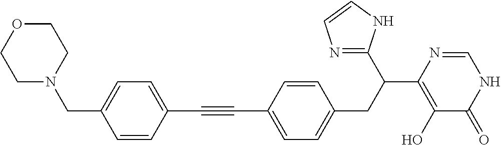 [SMILES: O=c1[nH]cnc(C(Cc2ccc(C#Cc3ccc(CN4CCOCC4)cc3)cc2)c2ncc[nH]2)c1O] |
| 222 | 6-(1-(1H-imidazol-5-yl)-3-(4-((4- (morpholinomethyl)phenyl)ethynyl) phenyl)propan-2-yl)-5- hydroxypyrimidin-4(3H)-one | 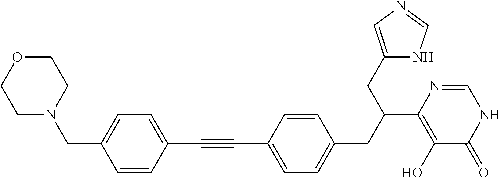 [SMILES: O=c1[nH]cnc(C(Cc2ccc(C#Cc3ccc(CN4CCOCC4)cc3)cc2)Cc2cnc[nH]2)c1O] |
| 223 | 2-((2-(5-hydroxy-6-oxo-1,6- dihydropyrimidin-4-yl)-3-(4-((4- (morpholinomethyl)phenyl)ethynyl) phenyl)propyl)amino)acetonitrile | 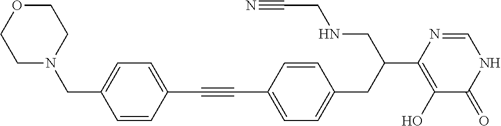 [SMILES: N#CCNCC(Cc1ccc(C#Cc2ccc(CN3CCOCC3)cc2)cc1)c1nc[nH]c(=O)c1O] |
| 224 | 3-((2-(5-hydroxy-6-oxo-1,6- dihydropyrimidin-4-yl)-3-(4-((4- (morpholinomethyl)phenyl)ethynyl) phenyl)propyl)amino)propanenitrile | 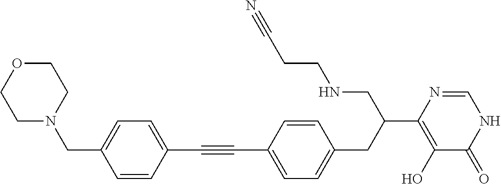 [SMILES: N#CCCNCC(Cc1ccc(C#Cc2ccc(CN3CCOCC3)cc2)cc1)c1nc[nH]c(=O)c1O] |
| 225 | 6-(1-(1,1-dioxido-1,3-thiazetidin-3- yl)-3-(4-((4- (morpholinomethyl)phenyl)ethynyl) phenyl)propan-2-yl)-5- hydroxypyrimidin-4(3H)-one | 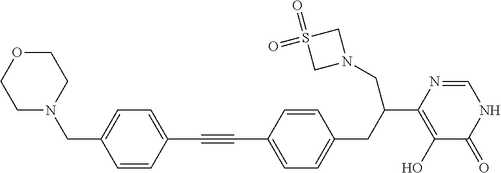 [SMILES: O=c1[nH]cnc(C(Cc2ccc(C#Cc3ccc(CN4CCOCC4)cc3)cc2)CN2CS(=O)(=O)C2)c1O] |
| 226 | N-(3-(4-((4-((2- ((dimethylamino)methyl)molpholino) methyl)phenyl)ethynyl)phenyl)-2-(5- hydroxy-6-oxo-1,6-dihydropyrimidin- 4-yl)propyl)-2-fluoroacetamide | 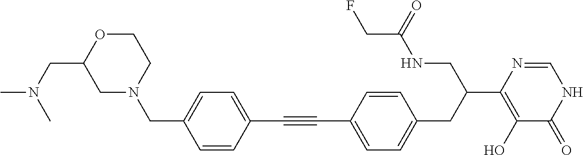 [SMILES: CN(C)CC1CN(Cc2ccc(C#Cc3ccc(CC(CNC(=O)CF)c4nc[nH]c(=O)c4O)cc3)cc2)CCO1] |
| 227 | 3-(4-((4-((2-oxa-5- azabicyclo[2.2.1]heptan-5- yl)methyl)phenyl)ethynyl)phenyl)-N- (2,2-difluoroethyl)-2-(5-hydroxy-6- oxo-1,6-dihydropyrimidin-4- yl)propanamide | 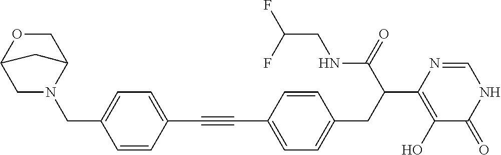 [SMILES: O=C(NCC(F)F)C(Cc1ccc(C#Cc2ccc(CN3CC4CC3CO4)cc2)cc1)c1nc[nH]c(=O)c1O] |
| 228 | N-(2,2-difluoroethyl)-2-(5-hydroxy-6- oxo-1,6-dihydropyrimidin-4-y1)-3-(4- ((4-(((3- methoxycyclobutyl)amino)methyl) phenyl)ethynyl)phenyl)propanamide | 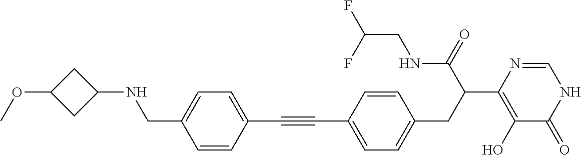 [SMILES: COC1CC(NCc2ccc(C#Cc3ccc(CC(C(=O)NCC(F)F)c4nc[nH]c(=O)c4O)cc3)cc2)C1] |
| 229 | 3-(4-((4-(2-amino-1- hydroxyethyl)phenyl)ethynyl)phenyl)- N-(2,2-difluoroethyl)-2-(5-hydroxy-6- oxo-1,6-dihydropyrimidin-4- yl)propanamide | 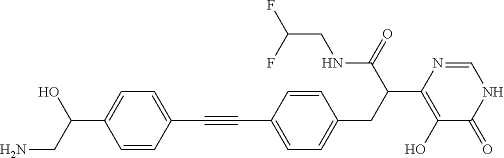 [SMILES: NCC(O)c1ccc(C#Cc2ccc(CC(C(=O)NCC(F)F)c3nc[nH]c(=O)c3O)cc2)cc1] |
| 230 | N-(2,2-difluoroethyl)-3-(4-((4-(1,2- dihydroxyethyl)phenyl)ethynyl) phenyl)-2-(5-hydroxy-6-oxo-1,6- dihydropyrimidin-4-yl)propanamide | 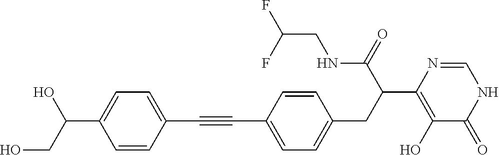 [SMILES: O=C(NCC(F)F)C(Cc1ccc(C#Cc2ccc(C(O)CO)cc2)cc1)c1nc[nH]c(=O)c1O] |
| 231 | 2,2-difluoro-N-(2-(5-hydroxy-6-oxo- 1,6-dihydropyrimidin-4-yl)-3-(4-((4- hydroxyethyl)phenyl)ethynyl)phenyl) propyl)acetamide | 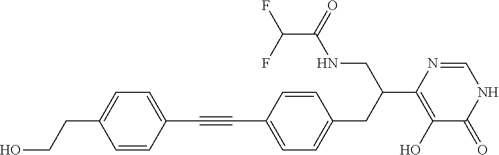 [SMILES: O=C(NCC(Cc1ccc(C#Cc2ccc(CCO)cc2)cc1)c1nc[nH]c(=O)c1O)C(F)F] |
| 232 | N-(3-(4-((4- (cyanomethyl)phenyl)ethynyl) phenyl)-2-(5-hydroxy-6-oxo-1,6- dihydropyrimidin-4-yl)propy1)- 2,2-difluoroacetamide | 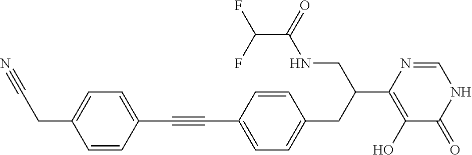 [SMILES: N#CCc1ccc(C#Cc2ccc(CC(CNC(=O)C(F)F)c3nc[nH]c(=O)c3O)cc2)cc1] |
| 233 | 3-(4-((4-((1H-tetrazol-1- yl)methyl)phenyl)ethynyl)phenyl)-N- (2,2-difluoroethyl)-2-(5-hydroxy-6- oxo-1,6-dihydropyrimidin-4- yl)propanamide | 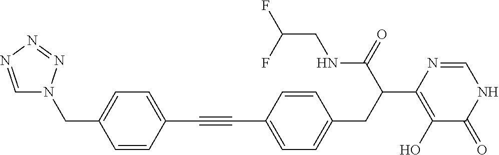 [SMILES: O=C(NCC(F)F)C(Cc1ccc(C#Cc2ccc(Cn3cnnn3)cc2)cc1)c1nc[nH]c(=O)c1O] |
| 234 | methyl 4-((4-(3-((2- fluoroethyl)amino)-2-(5-hydroxy-6- oxo-1,6-dihydropyrimidin-4- yl)propyl)phenyl)ethynyl)benzoate | 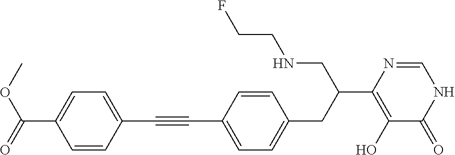 [SMILES: COC(=O)c1ccc(C#Cc2ccc(CC(CNCCF)c3nc[nH]c(=O)c3O)cc2)cc1] |
| 235 | N-(cyano methyl)-2-(5-hydroxy-6- oxo-1,6-dihydropyrimidin-4-yl)-3- (4-((4- (morpholinomethyl)phenyl)ethynyl) phenyl)propanamide | 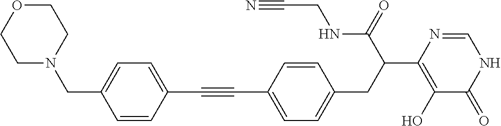 [SMILES: N#CCNC(=O)C(Cc1ccc(C#Cc2ccc(CN3CCOCC3)cc2)cc1)c1nc[nH]c(=O)c1O] |
| 236 | 2-(5-hydroxy-6-oxo-1,6- dihydropyrimidin-4-yl)-N-(2- methoxyethyl)-3-(4-((4- (morpholinomethyl)phenyl)ethynyl) phenyl)propanamide | 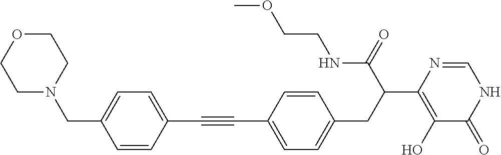 [SMILES: COCCNC(=O)C(Cc1ccc(C#Cc2ccc(CN3CCOCC3)cc2)cc1)c1nc[nH]c(=O)c1O] |
| 237 | 2-(5-hydroxy-6-oxo-1,6- dihydropyrimidin-4-yl)-3-(4-((4- (morpholinomethyl)phenyl)ethynyl) phenyl)-N-(oxetan-3-yl)propanamide | 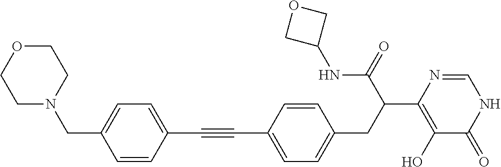 [SMILES: O=C(NC1COC1)C(Cc1ccc(C#Cc2ccc(CN3CCOCC3)cc2)cc1)c1nc[nH]c(=O)c1O] |
| 238 | N-(2,2-difluoroethyl)-2-(5-hydroxy-6- oxo-1,6-dihydropyrimidin-4-yl)-3-(4- ((4-((2- methylmorpholino)methyl)phenyl) ethynyl)phenyl)propanamide | 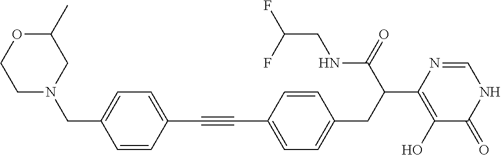 [SMILES: CC1CN(Cc2ccc(C#Cc3ccc(CC(C(=O)NCC(F)F)c4nc[nH]c(=O)c4O)cc3)cc2)CCO1] |
| 239 | 5-hydroxy-6-(1-(4-((4-((3- methoxypyrrolidin-1- yl)methyl)phenyl)ethynyl)phenyl)-3- ((2- (methylsulfonyl)ethyl)amino)propan- 2-yl)pyrimidin-4(3H)-one | 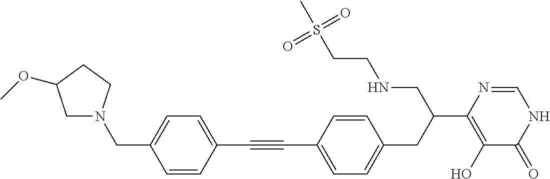 [SMILES: COC1CCN(Cc2ccc(C#Cc3ccc(CC(CNCCS(C)(=O)=O)c4nc[nH]c(=O)c4O)cc3)cc2)C1] |
| 240 | 6-(1-(3-fluoroazetidin-1-yl)-3-(4-((4- (morpholinomethyl)phenyl)ethynyl) phenyl)propan-2-yl)-5- hydroxypyrimidin-4(3H)-one | 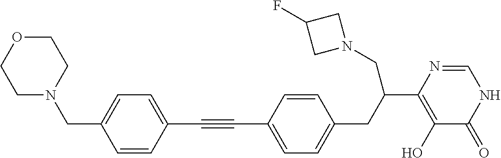 [SMILES: O=c1[nH]cnc(C(Cc2ccc(C#Cc3ccc(CN4CCOCC4)cc3)cc2)CN2CC(F)C2)c1O] |

In some embodiments, the heterocyclic LpxC inhibitory compound provided herein is selected from a compound provided in Table 3.

TABLE 3

| Compound | | | Mass |
|---|---|---|---|
| No. | Name | Structure | [M + H] |
| 241 | 5-hydroxy-6-(4-((4- (((tetrahydrofuran-3- yl)amino)methyl)phenyl) ethynyl)phenethyl)pyrimidin- 4(3H)-one | 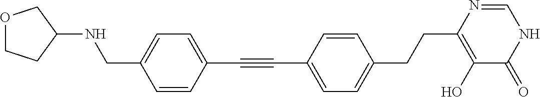 [SMILES: O=c1[nH]cnc(CCc2ccc(C#Cc3ccc(CNC4CCOC4)cc3)cc2)c1O] | 416.28 |
| 242 | 2-(4-((4-(2-(5-hydroxy-6-oxo- 1,6-dihydropyrimidin- 4- yl)ethyl)phenyl)ethynyl) phenyl)acetonitrile | 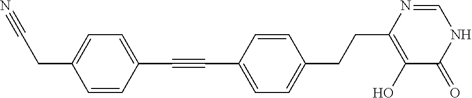 [SMILES: N#CCc1ccc(C#Cc2ccc(CCc3nc[nH]c(=O)c3O)cc2)cc1] | 356.23 |
| 243 | 5-hydroxy-6-(4-((4-(((3- hydroxycyclobutyl)amino) methyl)phenyl)ethynyl) phenethyl)pyrimidin-4(3H)- one | 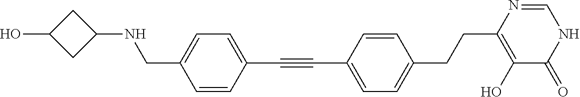 [SMILES: O=c1[nH]cnc(CCc2ccc(C#Cc3ccc(CNC4CC(O)C4)cc3)cc2)c1O] | 416.19 |
| 244 | 5-hydroxy-6-(4-((4- (((tetrahydro-2H-pyran-4- yl)amino)methyl)phenyl) ethynyl)phenethyl)pyrimidin- 4(3H)-one | 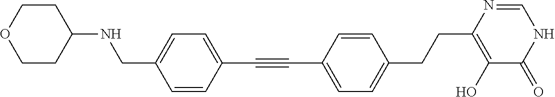 [SMILES: O=c1[nH]cnc(CCc2ccc(C#Cc3ccc(CNC4CCOCC4)cc3)cc2)c1O] | 430.33 |
| 245 | 5-hydroxy-6-(4-((4-(((2- methoxyethyl)amino) methyl)phenyl)ethynyl) phenethyl)pyrimidin-4(3H)- one | 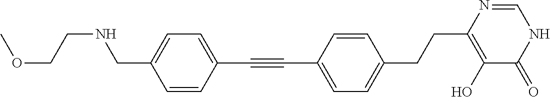 [SMILES: COCCNCc1ccc(C#Cc2ccc(CCc3nc[nH]c(=O)c3O)cc2)cc1] | 404.38 |
| 254 | 6-(4-((4-(1,2- dihydroxyethyl)phenyl) ethynyl)phenethyl)-5- hydroxypyrimidin-4(3H)- one | 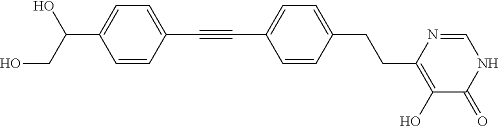 [SMILES: O=c1[nH]cnc(CCc2ccc(C#Cc3ccc(C(O)CO)cc3)cc2)c1O] | 377.12 |
| 255 | 6-(4-((4-(2-chloro-1- hydroxyethyl)phenyl) ethynyl)phenethyl)-5- hydroxypyrimidin-4(3H)- one | 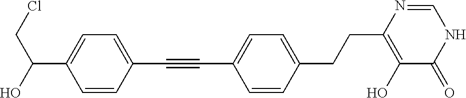 [SMILES: O=c1[nH]cnc(CCc2ccc(C#Cc3ccc(C(O)CCl)cc3)cc2)c1O] | 395.46 |
| 256 | N-(4-((4-(2-(5-hydroxy-6- oxo-1,6-dihydropyrimidin- 4- yl)ethyl)phenyl)ethynyl) benzyl)methanesulfonamide | 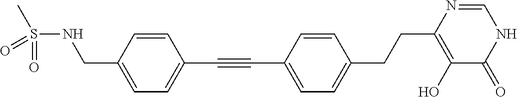 [SMILES: CS(=O)(=O)NCc1ccc(C#Cc2ccc(CCc3nc[nH]c(=O)c3O)cc2)cc1] | |
| 257 | (R)-2-hydroxy-N-(4-((4-(2- (5-hydroxy-6-oxo-1,6- dihydropyrimidin-4- yl)ethyl)phenyl)ethynyl) benzyl)-N-(pyrrolidin-3- yl)acetamide | 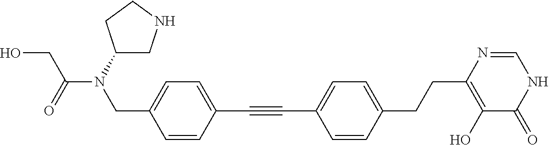 [SMILES: O=C(CO)N(Cc1ccc(C#Cc2ccc(CCc3nc[nH]c(=O)c3O)cc2)cc1)[C@@H]1CCNC1] | 473.2 |

### Preparation of Compounds

The compounds used in the chemical reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, Pa.), Aldrich Chemical (Milwaukee, Wis., including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, Pa.), Crescent Chemical Co. (Hauppauge, N.Y.), Eastman Organic Chemicals, Eastman Kodak Company (Rochester, N.Y.), Fisher Scientific Co. (Pittsburgh, Pa.), Fisons Chemicals (Leicestershire, UK), Frontier Scientific (Logan, Utah), ICN Biomedicals, Inc. (Costa Mesa, Calif.), Key Organics (Cornwall, U.K.), Lancaster Synthesis (Windham, N.H.), Maybridge Chemical Co. Ltd. (Cornwall, U.K.), Parish Chemical Co. (Orem, Utah), Pfaltz & Bauer, Inc. (Waterbury, Conn.), Polyorganix (Houston, Tex.), Pierce Chemical Co. (Rockford, Ill.), Riedel de Haen AG (Hanover, Germany), Spectrum Quality Product, Inc. (New Brunswick, N.J.), TCI America (Portland, Oreg.), Trans World Chemicals, Inc. (Rockville, Md.), and Wako Chemicals USA, Inc. (Richmond, Va.).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Alternatively, specific and analogous reactants can be identified through the indices of known chemicals and reactions prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the heterocyclic LpxC inhibitory compound described herein is P. H. Stahl & C. G. Wermuth “Handbook of Pharmaceutical Salts”, Verlag Helvetica Chimica Acta, Zurich, 2002.

### Pharmaceutical Compositions

In certain embodiments, the heterocyclic LpxC inhibitory compound as described herein is administered as a pure chemical. In other embodiments, the heterocyclic LpxC inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21st Ed. Mack Pub. Co., Easton, Pa. (2005)).

Provided herein is a pharmaceutical composition comprising at least one heterocyclic LpxC inhibitory compound as described herein, or a stereoisomer, pharmaceutically acceptable salt, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

One embodiment provides a pharmaceutical composition comprising a compound of Formula (I), (Ia), (IIa) or (IIIa), or a pharmaceutically acceptable salt thereof, and a pharmaceutically acceptable excipient.

In certain embodiments, the heterocyclic LpxC inhibitory compound as described by Formula (I), (Ia), (IIa) or (IIIa) is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

The dose of the composition comprising at least one heterocyclic LpxC inhibitory compound as described herein differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

### LpxC, Lipid A and Gram-Negative Bacteria

Metalloproteins influence a vast diversity of biological systems, biological processes, and diseases. For example, UDP-{3-O—[(R)-3-hydroxymyristoyl]}-N-acetylglucosamine deacetylase (LpxC) is an essential enzyme involved in the first committed step in lipid A biosynthesis for gram-negative bacteria. Lipid A is an essential component of the outer membrane of gram-negative bacteria. LpxC is a zinc(II)-dependent metalloenzyme, with two histidines and an aspartic acid residue bound to the zinc(II) ion. Structures of LpxC show the zinc(II) ion is bound to two water molecules, both of which have been implicated in the mechanism of the enzyme. LpxC is highly conserved across strains of gram-negative bacteria, making LpxC an attractive target to treat gram-negative infections.

In recent years, there has been an increase in resistant and multi-drug resistant strains of bacteria. Thus, there is a need for new antibiotics, especially with new mechanisms of action. There remains a need for metalloprotein modulators of LpxC useful in the field of therapeutics, diagnostics, and research.

One embodiment provides a method of inhibiting UDP-{3-O—[(R)-3-hydroxymyristoyl]}-N-acetylglucosamine deacetylase enzyme comprising contacting the enzyme with a compound of Formula (I), (Ia), (IIa) or (IIIa).

One embodiment provided herein is a pharmaceutical composition comprising a compound of Formula (I), (Ia), (IIa), or (IIIa) or a pharmaceutically acceptable salt thereof, and a pharmaceutically acceptable excipient. Another embodiment provided herein is a pharmaceutical composition comprising a compound of Formula (I) or a pharmaceutically acceptable salt thereof, and a pharmaceutically acceptable excipient. Another embodiment provided herein is a pharmaceutical composition comprising a compound of Formula (Ia) or a pharmaceutically acceptable salt thereof, and a pharmaceutically acceptable excipient. Another embodiment provided herein is a pharmaceutical composition comprising a compound of Formula (IIa) or a pharmaceutically acceptable salt thereof, and a pharmaceutically acceptable excipient. Another embodiment provided herein is a pharmaceutical composition comprising a compound of Formula (IIIa) or a pharmaceutically acceptable salt thereof, and a pharmaceutically acceptable excipient.

### Methods of Treatment

Disclosed herein are methods of treating disease wherein the inhibition of bacterial growth is indicated. Such disease includes gram-negative bacterial infection. In some embodiments, the method of treating a gram-negative bacterial infection in a patient in need thereof comprises administering to the patient a pharmaceutical composition comprising a compound of Formula (I), (Ia), (IIa) or (IIIa), or a pharmaceutically acceptable salt thereof, and a pharmaceutically acceptable excipient. In some embodiments, the gram-negative bacterial infection is selected from pneumonia, sepsis, cystic fibrosis, intra-abdominal infection, skin infections and urinary tract infection. In some embodiments, the gram-negative bacterial infection is a urinary tract infection (UTI), a hospital acquired/ventilator-associated pneumonia (HAP/VAP), or an intra-abdominal infection (IAI). In some embodiments, the gram-negative bacterial infection is selected from chronic urinary tract infections, complicated urinary tract infections, cystitis, pyelonephritis, urethritis, recurrent urinary tract infections, bladder infections, urethral infections, or kidney infections. In some embodiments, the compounds described herein are used for the treatment of chronic urinary tract infections. In some embodiments, the compounds described herein are used for the treatment of complicated urinary tract infections. In other embodiments, the compounds described herein are used for the treatment of complicated intra-abdominal infection. In some embodiments, the compounds described herein are used for the treatment of chronic intra-abdominal infection. In other embodiments, the compounds described herein are used for the treatment of hospital acquired pneumonia (HAP) or ventilator associated pneumonia (VAP). In some embodiments the administration is to treat an existing infection. In some embodiments the administration is provided as prophylaxis.

In some embodiments the heterocyclic LpxC inhibitory compound as described herein is used for treating conditions caused by the bacterial production of endotoxin and, in particular, by gram-negative bacteria and bacteria that use LpxC in the biosynthesis of lipopolysaccharide (LPS) or endotoxin. In some embodiments, the method of treating a condition caused by endotoxin or LPS in a patient in need thereof comprises administering to the patient a pharmaceutical composition comprising a compound of Formula (I), (Ia), (IIa) or (IIIa), or a pharmaceutically acceptable salt thereof, and a pharmaceutically acceptable excipient. In another embodiment, the heterocyclic LpxC inhibitory compounds as described herein are useful in the treatment of conditions that are caused or exacerbated by the bacterial production of lipid A and LPS or endotoxin, such as sepsis, septic shock, systemic inflammation, localized inflammation, chronic obstructive pulmonary disease (COPD) and acute exacerbations of chronic bronchitis (AECB). In some embodiments, the method of treating a condition caused by endotoxin or LPS in a patient in need thereof comprises administering to the patient a pharmaceutical composition comprising a compound of Formula (I), (Ia), (IIa) or (IIIa), or a pharmaceutically acceptable salt thereof, and a pharmaceutically acceptable excipient, wherein the condition caused by endotoxin or LPS is selected from sepsis, septic shock, systemic inflammation, localized inflammation, chronic obstructive pulmonary disease (COPD) and acute exacerbations of chronic bronchitis (AECB).

In other embodiments, the compounds of the disclosure can be used for the treatment of a serious or chronic respiratory tract infection or complicated urinary tract infections including serious lung and nosocomial infections such as those caused by Enterobacter aerogenes, Enterobacter cloacae, Escherichia coli, Klebsiella pneumoniae, Klebsiella oxytoca, Proteus mirabilis, Serratia marcescens, Stenotrophomonas maltophilia, Pseudomonas aeruginosa, Burkholderia cepacia, Acinetobacter baumannii, Alcaligenes xylosoxidans, Flavobacterium meningosepticum, Providencia sluarlii and Citrobacter freundi, Haemophilus influenzae, Legionella species, Moraxella catarrhalis, Enterobacter species, Acinetobacter species, Klebsiella species, Burkholderia species and Proteus species, and infections caused by other bacterial species such as Neisseria species, Shigella species, Salmonella species, Helicobacler pylori, Vibrionaceae and Bordetella species as well as the infections caused by a Brucella species, Francisella tularensis and/or Yersinia pestis.

In one embodiment provided herein is a method of treating a gram-negative bacterial infection in a patient in need thereof comprising administering to the patient a pharmaceutical composition comprising a compound of Formula (I), (Ia), (IIa), or (IIIa), or a pharmaceutically acceptable salt thereof, and a pharmaceutically acceptable excipient.

One embodiment provides a method wherein the gram-negative bacterial infection is selected from pneumonia, sepsis, cystic fibrosis, intra-abdominal infection, skin infection and urinary tract infection.

One embodiment provides a method wherein the gram-negative bacterial infection is selected from chronic urinary tract infection, complicated urinary tract infection, cystitis, pyelonephritis, urethritis, recurrent urinary tract infections, bladder infections, urethral infections and kidney infections.

One embodiment provides a method wherein the gram-negative bacterial infection is chronic urinary tract infections. One embodiment provides a method wherein the gram-negative bacterial infection is complicated urinary tract infections. One embodiment provides a method wherein the administration is to treat an existing infection. One embodiment provides a method wherein the administration is provided as prophylaxis.

One embodiment provides a method of treating a gram-negative bacterial infection in a patient in need thereof comprising administering to the patient a pharmaceutical composition comprising a compound Formula (I), Formula (Ia), Formula (IIa), Formula (IIIa), or a pharmaceutically acceptable salt thereof, and a pharmaceutically acceptable excipient. In one embodiment, the gram-negative bacterial infection is selected from pneumonia, sepsis, cystic fibrosis, intra-abdominal infection, skin infection and urinary tract infection. In another embodiment, the gram-negative bacterial infection is selected from chronic urinary tract infection, complicated urinary tract infection, cystitis, pyelonephritis, urethritis, recurrent urinary tract infections, bladder infections, urethral infections and kidney infections. In one embodiment, the gram-negative bacterial infection is chronic urinary tract infections. In another embodiment, the gram-negative bacterial infection is complicated urinary tract infections. In one embodiment, the administration is to treat an existing infection. In an additional embodiment, the administration is provided as prophylaxis.

Other embodiments and uses will be apparent to one skilled in the art in light of the present disclosures. The following examples are provided merely as illustrative of various embodiments and shall not be construed to limit the invention in any way.

### EXAMPLES

### I. Chemical Synthesis

Unless otherwise noted, reagents and solvents were used as received from commercial suppliers. Anhydrous solvents and oven-dried glassware were used for synthetic transformations sensitive to moisture and/or oxygen. Yields were not optimized. Reaction times are approximate and were not optimized. Column chromatography and thin layer chromatography (TLC) were performed on silica gel unless otherwise noted. Spectra are given in ppm (δ) and coupling constants, J are reported in Hertz. For proton spectra the solvent peak was used as the reference peak.

The following abbreviations and terms have the indicated meanings throughout:
AcOH=acetic acid
B₂pin₂=bis(pinacolato)diboron
Boc=tert-butoxycarbonyl
DCC=dicyclohexylcarbodiimide
DIEA=N,N-diisopropylethylamine
DMAP=4-dimethylaminopyridine
EDC=1-ethyl-3-(3-dimethylaminopropyl) carbodiimide
eq=equivalent(s)
Et=ethyl
EtOAc or EA=ethyl acetate
EtOH=ethanol
g=gram
h or hr=hour
HBTU=O-(benzotriazol-1-yl)-N,N,N′,N′-tetramethyluronium hexafluorophosphate
HOBt=hydroxybenzotriazole
HPLC=high pressure liquid chromatography
kg or Kg=kilogram
L or l=liter
LC/MS=LCMS=liquid chromatography-mass spectrometry
LRMS=low resolution mass spectrometry
m/z=mass-to-charge ratio
Me=methyl
MeOH=methanol
mg=milligram
min=minute
mL=milliliter
mmol=millimole
NaOAc=sodium acetate
PE=petroleum ether
Ph=phenyl
Prep=preparative
quant.=quantitative
RP-HPLC=reverse phase-high pressure liquid chromatography
rt or RT=room temperature
THF=tetrahydrofuran
UV=ultraviolet

### Example 1

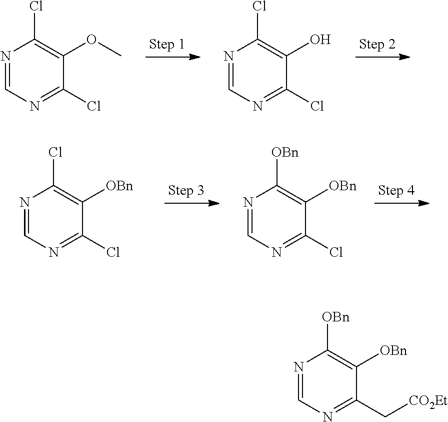

Step 1—To a stirred solution of 4,6-dichloro-5-methoxypyrimidine (50 g, 0.27 mol) in dichloroethane (800 mL) at 0° C., anhydrous aluminum chloride (50.48 g, 0.411 mol) was added in a single portion. The reaction mixture was stirred vigorously at 50° C. for 6 h. After completion, the mixture was cooled to 0° C. and aqueous HCl solution (1 M, 400 mL) was added slowly followed by addition of MeOH (100 mL). The mixture was stirred vigorously at room temperature for 10 min, diluted with water and extracted with EtOAc. The combined organic layers were washed with satd. NaCl, dried over Na₂SO₄, filtered and concentrated to afford 4, 6-dichloropyrimidin-5-ol (41 g, 91.11%). UPLC=Calculated for C₄H₂Cl₂N₂O is 164.97, Observed=165.9.

Step 2—To a stirred solution of 4, 6-dichloropyrimidin-5-ol (6.5 g, 35.50 mmol) in DMF (120 mL), benzyl bromide (8.42 mL, 70.90 mmol) was added followed by the addition of potassium carbonate (14.70 g, 106.36 mmol). The reaction mixture was stirred at 60° C. for 1 h. The mixture was concentrated and the residue was partitioned between EtOAc (150 mL) and ice cold water (75 mL). The aqueous layer was further extracted with EtOAc (2×100 mL). The organic layers were combined and washed with satd. NaCl, dried over Na₂SO₄, filtered and concentrated. The residue was purified by flash chromatography on silica gel (230-400 mesh, 10% EtOAc in pet. ether) to afford 5-(benzyloxy)-4, 6-dichloropyrimidine (9.5 g, 94%). LCMS=Calculated for C₁₁H₈Cl₂N₂O is 255.10, Observed=256.1.

Step 3—To a solution of benzyl alcohol (3.8 g, 0.0352 mol) in THF (100 mL), NaH (60% in mineral oil, 1.4 g, 0.0352 mol) was added at 0° C. and stirred for 30 min. To this a solution of 5-(benzyloxy)-4, 6-dichloropyrimidine (9 g, 0.0352 mol) in THF (20 mL) was added at 0° C. and stirred for 30 min. After completion of the reaction, the reaction mixture was diluted with water and extracted with EtOAc (2×100 mL). The organic layers were combined and washed with satd. NaCl, dried over Na₂SO₄, filtered and concentrated under reduced pressure to afford 4, 5-bis (benzyloxy)-6-chloropyrimidine (11 g, 95.6%). UPLC=Calculated for C₁₈H₁₅ClN₂O₂ is 326.78, Observed=327.6.

Step 4—To a solution of 4,5-bis (benzyloxy)-6-chloropyrimidine (10 g, 0.0306 mol) in dry toluene (20 mL), diethyl malonate (29.4 g, 0.183 mol) and t-BuOK (10.29 g, 0.0918 mol) were added and refluxed at 120° C. for 3 h. After completion of the reaction, the reaction mixture was diluted with water and extracted with EtOAc (2×100 mL). The combined organic layers were washed with satd. NaCl, dried over anhydrous Na₂SO₄, filtered and concentrated under reduced pressure. The residue was purified by flash chromatography on silica gel (230-400 mesh, 15% EtOAc in pet. ether) to afford ethyl 2-(5, 6-bis (benzyloxy) pyrimidin-4-yl) acetate (3 g, 26%). UPLC=Calculated for C₂₂H₂₂N₂O₄ is 378.43, Observed=379.4.

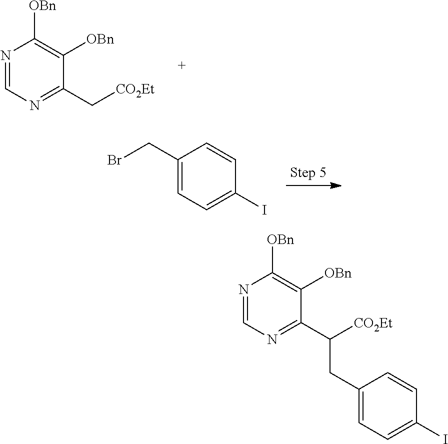

Step—5 To a solution of ethyl 2-(5, 6-bis (benzyloxy) pyrimidin-4-yl) acetate (3 g, 0.0079 mol) in DMF (30 mL) NaH (60%, 0.317 g, 0.0079 mol) was added at 0° C. and stirred for 30 min. Then 4-Iodo benzyl bromide (2.35 g, and 0.0079 mol) was added and stirred at 0° C. for 2 h. After completion of the reaction, the reaction mixture was quenched with sat. NH₄Cl and diluted with water and extracted with EtOAc (2×100 mL). The combined organic layers were washed with satd. NaCl, dried over Na₂SO₄, filtered and concentrated under vacuum. The crude product was purified by column chromatography on silica gel (230-400 mesh, 10% EtOAc in pet. ether) to afford ethyl 2-(5, 6-bis (benzyloxy) pyrimidin-4-yl)-3-(4-iodophenyl) propanoate (4.4 g, 93.6%). LCMS=Calculated for C₂₉H₂₇IN₂O₄ is 594.45, Observed=595.33.

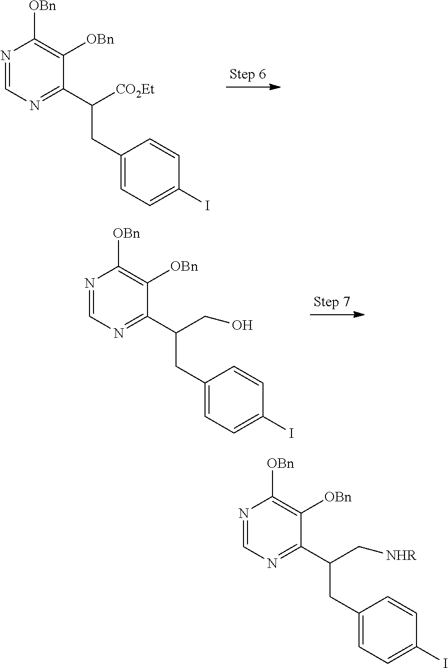

Step 6—Solution of ethyl 2-(5, 6-bis (benzyloxy) pyrimidin-4-yl) acetate (12 g, 0.0201 mol) in THF (120 mL) was cooled to 0° C., to this cooled solution DIBAL-H (60.5 mL, 0.0605 mol) was added slowly dropwise and allowed gradually to stir at 25° C. for 3 h. After completion, the reaction mixture was quenched with satd. NH₄Cl (300 mL) at −30° C. and stirred for 30 min. The solution was further acidified with 3 M HCl (2.0 mL) and stirred for 10 minutes. The reaction mass was filtered on a celite bed and the bed was washed with EtOAc (500 mL). The layers were separated and the organic layers were washed with brine solution, dried over Na₂SO₄, filtered and concentrated to get crude product. The compound was further purified on a column chromatography on silica gel (230-400 mesh, 50% EtOAc in hexanes to afford 2-(5, 6-bis (benzyloxy) pyrimidin-4-yl)-3-(4-iodophenyl) propan-1-ol (9.2 g, 83%). LCMS=Calculated for C₂₇H₂₅IN₂O₃ is 552.41, Observed=553.3.

Step 7—To a stirred solution of 2-(5, 6-bis (benzyloxy) pyrimidin-4-yl)-3-(4-iodophenyl) propan-1-ol (0.500 g, 0.00096 mol) in DCM (10 mL), Dess martin periodinane (0.845 g, 0.0020 mol) was added and stirred for a period of 1 h. After completion, the reaction mixture was quenched with satd. NaHCO₃ solution and extracted with DCM (2×20 mL). The organic layers were combined, washed with satd. NaCl (50 mL), dried over Na₂SO₄ and concentrated to dryness under vacuum to get the desired crude aldehyde 2-(5,6-bis(benzyloxy)pyrimidin-4-yl)-3-(4-iodophenyl)propanal (0.460 g, 92%). [Note: The water bath temp should not exceed 35 OC during evaporation]. The compound was used as such without further purification as soon as possible due to stability issues. The was dissolved in minimal DCM, followed by the addition of amine, with MeOH if an HCl salt, added along with DCM to enable the solubility. After 45 min, an additional portion of MeOH was added, along with NaCNBH₃ and the stirring was prolonged for an additional 2 h. After completion, the reaction mixture was evaporated to dryness. The compound was extracted in EtOAc The organic layer was washed with 6 M HCl. The organic layer was discarded and the HCl layer was neutralized to pH=8 using 3 M NaOH and re-extracted with EtOAc. The organic layer was then washed with satd. NaCl, dried over Na₂SO₄ and concentrated to dryness to get the crude product. The compound was further purified on a column chromatography using silica gel and eluting with MeOH (5%) in DCM to get the desired product.

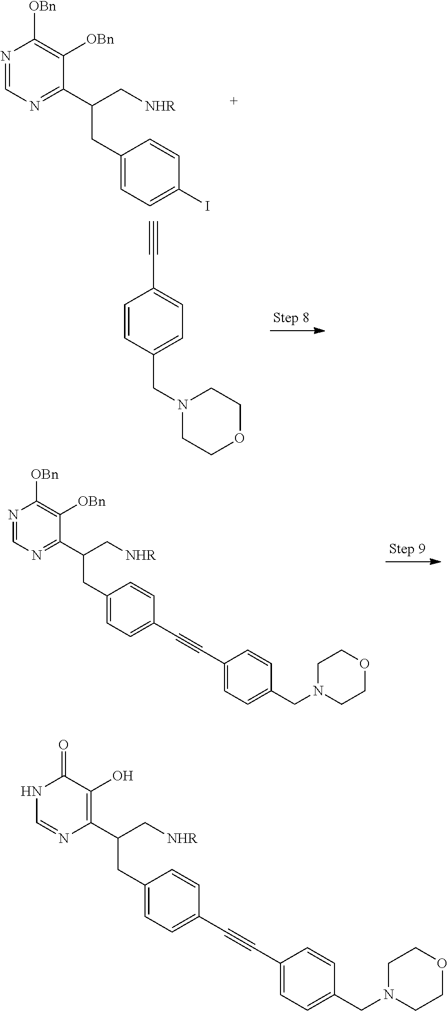

### Synthesis of 4-(4-ethynylbenzyl) morpholine

To a stirred solution of 4-ethynylbenzaldehyde (5.0 g, 0.0384 mol) in MeOH (20 mL), morpholine (5.0 g, 5.0 mL, 0.058 mol) was added along with 0.1 mL of AcOH. The reaction was stirred for 1 h until a homogenous solution was visible. To the reaction mixture NaCNBH₃ (2.90 g, 1.2 eq, 0.046 mol) was added and stirred for a period of 2 h. After completion, the reaction mixture was concentrated to dryness under vacuum. The reaction mixture was partitioned between water (50 mL) and EtOAc (100 mL). The water layer was slightly acidified with dil. HCl for a better separation. The aqueous layer was further extracted with EtOAc (100 mL). The organic layers were combined washed over satd. NaCl (50 mL), dried over Na₂SO₄ and concentrated to dryness to get a crude product. The crude product was further purified on a column chromatography on silica gel to get the desired product (6.7 g, 87%). LCMS=Calculated for C₁₃H₁₅NO is 201.27, observed=202.27.

Step 8—To a solution of the product of Step 7 was added 4-(4-ethynylbenzyl) morpholine and Et₃N. The reaction mixture was bubbled with nitrogen for 10 min, PdCl₂(PPh₃)₂ was added followed by CuI and the reaction mixture was stirred at 100° C. for 1 h. After completion of the reaction, solvent was removed under reduced pressure and the reaction mixture was dissolved in water and extracted with EtOAc (2×50 mL). The combined organic layers were washed with satd. NaCl (50 mL), dried over Na₂SO₄, filtered and concentrated and the crude product was purified by column chromatography on silica gel (230-400 mesh, 15% MeOH in DCM) to get the desired product.

Step 9—To a solution of the product of Step 8 in DCM (5 mL), BCl₃ was added and stirred at 25° C. for 2 h. Then the reaction mixture was quenched with methanol and neutralized to pH=7-8 and concentrated under reduced pressure. The crude product was purified by reverse phase HPLC to afford the desired product as a formate salt.

### Example 2

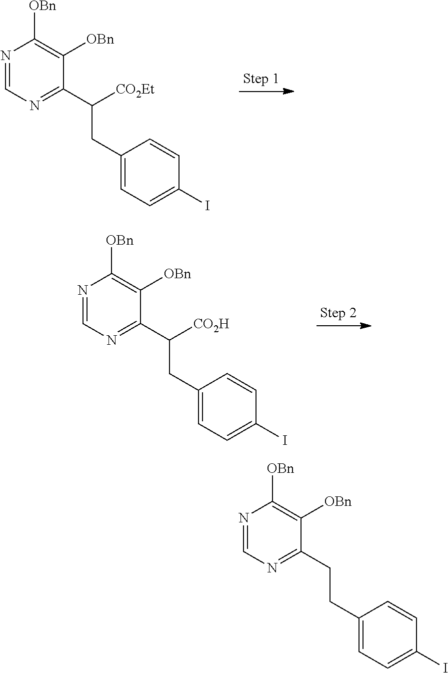

Step 1—To a solution of 2-(5, 6-bis (benzyloxy) pyrimidin-4-yl)-3-(4-iodophenyl) propanoate (1.4 g, 0.0029 mol) in EtOH:THF (1:1) (10 mL), water (10 mL) was added followed by NaOH (0.59 g, 0.0148 mol) and the reaction mixture was stirred for 4 h. After completion of the reaction, the reaction mixture was diluted with water and extracted with diethyl ether (100 mL). The organic layer was separated and the aqueous layer was acidified with 1.5N HCl to pH 6-7 and extracted with EtOAc (2×50 mL). The combined organic layers were washed with satd. NaCl, dried over Na₂SO₄, filtered and concentrated under reduced pressure to afford 2-(5,6-bis(benzyloxy)pyrimidin-4-yl)-3-(4-iodophenyl)propanoic acid (1.2 g, 91.6%). UPLC=Calculated for C₂₇H₂₃IN₂O₄ is 566.40, Observed=567.20

Step 2—The compound 2-(5,6-bis(benzyloxy)pyrimidin-4-yl)-3-(4-iodophenyl)propanoic acid (1 g, 0.0017 mol) obtained from previous step is heated at 67° C. in a water bath under vacuum for a period of 1 h. After completion, the reaction mixture was diluted with DCM and purified on a column chromatography using silica gel using EtOAc and hexanes to get the desired compound 4,5-bis(benzyl oxy)-6-(4-iodophenethyl)pyrimidine (0.770 g, 87%). LCMS=Calculated for C₂₆H₂₃IN₂O₃ is 522.39, observed=523.19.

### Example 3

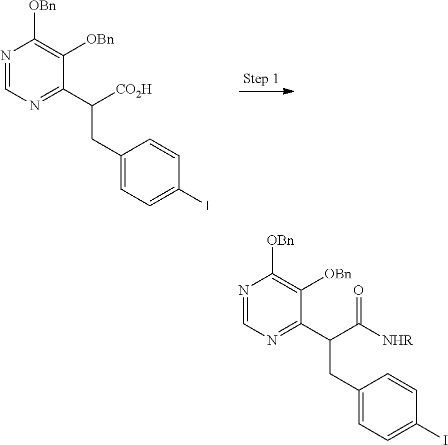

Step 1—To a stirred solution of 2-(5,6-bis(benzyloxy)pyrimidin-4-yl)-3-(4-iodophenyl)propanoic acid in DCM, amine was added followed by EDC.HCl and HOBT and the reaction mixture was stirred for a period of 4 h. After completion, the reaction mixture was diluted with DCM and washed with satd. NaHCO₃ (30 mL) solution followed by satd. NaCl and dried further over Na₂SO₄ and concentrated under vacuum to get a crude product. The crude product was purified on a column chromatography on a silica gel using EtOAc (90%) in hexanes to get the desired product.

### Example 4

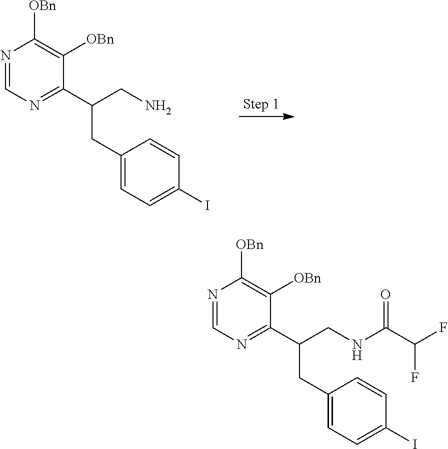 [SMILES: NCC(Cc1ccc(I)cc1)c1ncnc(OCc2ccccc2)c1OCc1ccccc1 ; O=C(NCC(Cc1ccc(I)cc1)c1ncnc(OCc2ccccc2)c1OCc1ccccc1)C(F)F]

Step 1—A solution of 2-(5, 6-bis (benzyloxy) pyrimidin-4-yl)-3-(4-iodophenyl) propan-1-amine, a carboxylic acid, HATU and DIPEA were added at 25° C. and the reaction mixture was stirred for 4 h. After completion of the reaction, the reaction mixture was diluted with water and extracted with EtOAc. The combined organic layers were dried over Na₂SO₄, filtered, concentrated under reduced pressure. The crude product was purified by column chromatography on silica gel (230-400 mesh, 50-70% EtOAc in pet. ether) to afforded the desired product.

### II. Biological Evaluation

### Example 1: In Vitro Assays to Screen Compounds and Metalloprotein Modulators Bacterial Susceptibility Testing

Minimal inhibitory concentrations (MIC) were determined by the broth microdilution method in accordance with the Clinical and Laboratory Standards Institute (CLSI) guidelines. In brief, organism suspensions were adjusted to a 0.5 McFarland standard to yield a final inoculum between 3×10⁵ and 7×10⁵ colony-forming units (CFU)/mL. Drug dilutions and inocula were made in sterile, cation adjusted Mueller-Hinton Broth (Beckton Dickinson). An inoculum volume of 100 μL was added to wells containing 100 μL of broth with 2-fold serial dilutions of drug. All inoculated microdilution trays were incubated in ambient air at 35° C. for 18-24 h. Following incubation, the lowest concentration of the drug that prevented visible growth (OD600 nm<0.05) was recorded as the MIC. Performance of the assay was monitored by the use of laboratory quality-control strains and levofloxacin, a compound with a defined MIC spectrum, in accordance with CLSI guidelines.

Exemplary in vitro assay data against select bacteria for compounds in embodiments of the disclosure is provided in Table 4.

TABLE 4

| Compound | E. coli | K. pneumoniae | S. aureus |
|---|---|---|---|
| No. | MIC | MIC | MIC |
| 1 | A | B | D |
| 2 | A | B | D |
| 3 | A | B | D |
| 4 | B | D | D |
| 5 | B | D | D |
| 6 | C | D | D |
| 7 | C | D | D |
| 8 | D | D | D |
| 9 | D | D | D |
| 10 | B | C | D |
| 11 | D | D | D |
| 12 | A | B | D |
| 13 | B | B | D |
| 14 | B | C | D |
| 15 | A | B | D |
| 16 | B | C | D |
| 17 | B | C | D |
| 18 | B | B | D |
| 19 | B | C | D |
| 20 | B | C | D |
| 21 | B | C | D |
| 241 | A | A | D |
| 185 | A | A | D |
| 186 | A | A | D |
| 187 | A | A | D |
| 245 | A | B | D |
| 244 | A | A | D |
| 188 | A | B | D |
| 42 | D | D | D |
| 44 | D | D | D |
| 45 | D | D | D |
| 46 | D | D | D |
| 246 | D | D | |
| 247 | B | C | |
| 248 | D | D | |
| 249 | A | D | |
| 250 | A | D | |
| 251 | D | D | |

Note:
Microbiological activity data are designated within the following ranges:
A: ≤1 μg/mL
B: >1 μg/mL to ≤8.0 μg/mL
C: >8.0 μg/mL to ≤32 μg/mL
D: >32 μg/mL

### TLR4 Assay

The TLR4 assay measures activity of diluted filter-sterilized A. baumannii culture supernatants. The assay was performed as described in Lin et al, Mbio, 2012. Briefly, A. baumannii was passaged to mid log-phase in the presence of Forge LpxC inhibitors. Culture supernatants were harvested, filter-sterilized and diluted 1:1000. TLR4 activity of the supernatants was tested using HEK-Blue-hTLR4 cells (Invivogen) that carry NF-kB and AP-1 driven SEAP (secreted embryonic alkaline phosphatase) reporter gene. Table 5 shows the suppression of TLR-4 activity by representative compounds relative to a DMSO control (100% activity).

Table 5.

Exemplary TLR4 assay data (% TLR-4 activity) for compounds in embodiments of the disclosure.

TABLE 5

| Compound No. | 4 ug/mL | 8 ug/mL | 16 ug/mL |
|---|---|---|---|
| 15 | C | C | A |
| 58 | A | A | A |
| 1 | A | A | A |
| 3 | C | B | B |
| 2 | A | A | A |
| 102 | B | A | A |
| 187 | C | C | B |
| 14 | D | D | D |
| 60 | D | D | D |
| 57 | D | C | B |
| 153 | D | C | A |
| 55 | D | B | B |
| 106 | C | C | B |
| 215 | B | B | A |
| 195 | C | A | A |

Note:
TLR-4 activity is designated within the following ranges:
A: ≤20%
B: >20% to ≤50%
C: >50% to ≤80%
D: >80%

LpxC Binding Assay

IC₅₀ values against E. coli LpxC were determined using a Raipid Fire MS assay as previously described J. Med. Chem. 2012, 55, 1662-1670.

Table 6.

Exemplary in vitro assay data against E. coli LpxC for compounds in embodiments of the disclosure.

TABLE 6

| Compound No. | E. coli LpxC IC₅₀ |
|---|---|
| 198 | D |
| 207 | C |
| 244 | D |
| 195 | D |
| 58 | B |
| 243 | B |
| 105 | C |
| 12 | B |
| 209 | B |
| 80 | D |
| 106 | D |
| 153 | B |
| 56 | B |
| 109 | B |
| 152 | D |
| 15 | B |
| 55 | B |
| 165 | A |
| 3 | B |
| 154 | C |
| 108 | B |
| 87 | C |
| 1 | A |
| 149 | A |
| 79 | D |
| 57 | D |
| 218 | D |
| 2 | B |
| 215 | D |
| 114 | C |
| 98 | A |
| 248 | D |
| 250 | A |

Note:
IC₅₀ data are designated within the following ranges
A: ≤10 nM
B: >10 nM to ≤50 nM
C: >50 nM to ≤100 nM
D: >100 nM to 1 μM

### III. Preparation of Pharmaceutical Dosage Forms

### Example 1: Oral Capsule

The active ingredient is a compound of Formula (I), or a pharmaceutically acceptable salt thereof. A capsule for oral administration is prepared by mixing 1-1000 mg of active ingredient with starch or other suitable powder blend. The mixture is incorporated into an oral dosage unit such as a hard gelatin capsule, which is suitable for oral administration.

We claim:

1. A compound, or a pharmaceutically acceptable salt thereof, having the structure of Formula (I): 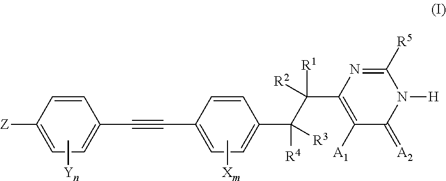
wherein,
n is 0-4;
m is 0-4;
A₁ is OH or SH;
A₂ is O or S;
R¹, R², R³ and R⁴ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹);
or R¹ and R² are taken together to form an optionally substituted alkenyl, ═NR¹¹, oxo, or thioxo;
or R¹ and R² are joined to form an optionally substituted carbocyclyl or optionally substituted heterocyclyl;
or R³ and R⁴ are taken together to form an optionally substituted alkenyl; ═NR¹¹, oxo, or thioxo;
or R³ and R⁴ are joined to form an optionally substituted carbocyclyl or optionally substituted heterocyclyl;
R⁵ is H, halogen, optionally substituted alkyl, hydroxyl, alkoxyl, cyano, amino, or nitro;
X and Y are each independently optionally substituted alkyl, optionally substituted alkenyl, optionally substituted alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN;
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2;
and wherein if R¹ is unsubstituted (C₁-C₄ alkylene)-OH, then (i) at least one of R², R³, or R⁴ is not H; or (ii) n is 1-4 and m is 0-4; or (iii) n is 0-4 and m is 1-4;
and wherein if R² is unsubstituted (C₁-C₄ alkylene)-OH, then (i) at least one of R¹, R³, or R⁴ is not H; or (ii) n is 1-4 and m is 0-4; or (iii) n is 0-4 and m is 1-4.

2. The compound of claim 1, or a pharmaceutically acceptable salt thereof, wherein:
R¹, R², R³ and R⁴ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
or R¹ and R² are taken together to form an optionally substituted alkenyl, ═NR¹¹, oxo, or thioxo;
or R³ and R⁴ are taken together to form an optionally substituted alkenyl; ═NR¹¹, oxo, or thioxo; and
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₁-C₄ alkylene)-SO₂N(R¹³)₂, or —SO₂(R¹³);
wherein R¹ and R² are each not a hydroxy substituted alkyl.

3. A compound, or a pharmaceutically acceptable salt thereof, having the structure of Formula (Ia): 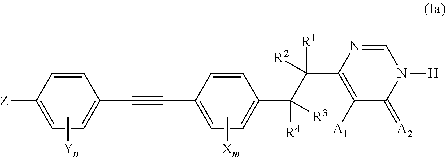
wherein,
n is 0, 1, or 2;
m is 0, 1, or 2;
A₁ is OH or SH;
A₂ is O or S;
R¹ and R³ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, optionally substituted (C₁-C₄ alkylene)N(R¹¹)—PO (optionally substituted C₁-C₄ alkyl)₂, optionally substituted (C₁-C₄ alkylene)-SO₂R¹¹, optionally substituted (C₁-C₄ alkylene)-O—SO₂R¹¹, or optionally substituted (C₀-C₄ alkylene)-C(═N—OR¹¹)(R¹¹);
R² is H or optionally substituted C₁-C₃ alkyl, provided that if R¹ is H, then R² is H or unsubstituted C₁-C₃ alkyl; or R¹ and R² are taken together to form an optionally substituted alkenyl; or R¹ and R² are joined to form an optionally substituted carbocyclyl or optionally substituted heterocyclyl;
R⁴ is H or optionally substituted C₁-C₃ alkyl; or R³ and R⁴ are taken together to form an optionally substituted alkenyl; or R³ and R⁴ are joined to form an optionally substituted carbocyclyl or optionally substituted heterocyclyl;
X is halogen, hydroxy, or optionally substituted C₁-C₃ alkyl;
Y is halogen, hydroxy, or optionally substituted C₁-C₃ alkyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₀-C₄ alkylene)-N(R¹⁴)CO₂(R¹³), optionally substituted (C₁-C₄ alkylene)-SO₂N(R¹³)₂, optionally substituted (C₀-C₄ alkylene)-N(R¹⁴)SO₂N(R¹³)₂, or optionally substituted (C₀-C₄ alkylene)-SO₂(R¹³), optionally substituted (C₀-C₄ alkylene)-COR¹³, optionally substituted (C₀-C₄ alkylene)-CO₂R¹³, optionally substituted (C₀-C₄ alkylene)-CON(R¹³)₂, optionally substituted (C₀-C₄ alkylene)-N(R¹⁴)—COR¹³, optionally substituted (C₀-C₄ alkylene)-N(R¹⁴)—SO₂R¹³, optionally substituted (C₀-C₄ alkylene)-O—SO₂N(R¹³)₂, or optionally substituted (C₀-C₄ alkylene)-C(═N—OR¹³)(R¹³);
L is a bond, optionally substituted C₁-C₄ alkylene, —C(O)—, or —SO₂—;
G is optionally substituted alkyl, optionally substituted alkenyl, optionally substituted alkynyl, halo, fluoroalkyl, oxo, thioxo, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —N(R¹³)₂, —OR¹³, or —CN
each R¹¹ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
each R¹² is independently selected from H or optionally substituted alkyl;
each R¹³ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl; and
each R¹⁴ is independently selected from H or optionally substituted alkyl;
wherein if R¹, R², R³, and R⁴ are H, then n is 1 or 2 and m is 0, 1, or 2; or n is 0, 1, or 2 and m is 1 or 2;
and wherein if R¹ is unsubstituted (C₁-C₄ alkylene)-OH, then (i) at least one of R², R³, or R⁴ is not H; or (ii) n is 1 or 2 and m is 0, 1, or 2; or (iii) n is 0, 1, or 2 and m is 1 or 2;
and wherein if R² is unsubstituted (C₁-C₄ alkylene)-OH, then (i) at least one of R¹, R³, or R⁴ is not H; or (ii) n is 1 or 2 and m is 0, 1, or 2; or (iii) n is 0, 1, or 2 and m is 1 or 2.

4. The compound of claim 3, or a pharmaceutically acceptable salt thereof, wherein:
R¹ and R³ are each independently H, unsubstituted alkyl, optionally substituted aryl, optionally substituted aralkyl, optionally substituted heteroaryl, optionally substituted heteroaralkyl, optionally substituted heterocyclyl, optionally substituted heterocyclalkyl, —COR¹¹, —CON(R¹¹)₂, optionally substituted (C₁-C₄ alkylene)-CN, optionally substituted (C₁-C₄ alkylene)-OR¹¹, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, optionally substituted (C₁-C₄ alkylene)N(R¹²)—CON(R¹¹)₂, optionally substituted (C₁-C₄ alkylene)N(R¹²)—SO₂N(R¹¹)₂, optionally substituted (C₁-C₄ alkylene)-O—SO₂N(R¹¹)₂, or optionally substituted (C₁-C₄ alkylene)N(R¹¹)—PO (optionally substituted C₁-C₄ alkyl)₂;
R² is H or optionally substituted C₁-C₃ alkyl, provided that if R¹ is H, then R² is H or unsubstituted C₁-C₃ alkyl; or R¹ and R² are taken together to form an optionally substituted alkenyl;
R⁴ is H or optionally substituted C₁-C₃ alkyl; or R³ and R⁴ are taken together to form an optionally substituted alkenyl;
Z is H, -L-G, optionally substituted (C₁-C₄ alkylene)-OCON(R¹³)₂, optionally substituted (C₁-C₄ alkylene)-N(R¹⁴)CON(R¹³)₂, optionally substituted (C₁-C₄ alkylene)-SO₂N(R¹³)₂, or —SO₂(R¹³); and
G is optionally substituted alkyl, optionally substituted heterocyclyl, optionally substituted heteroaryl, —N(R¹³)₂, —OR¹³, or —CN;
wherein R¹ and R² are each not a hydroxy substituted alkyl.

5. The compound of either of claim 3 or 4, or a pharmaceutically acceptable salt thereof, wherein A¹ is OH.

6. The compound of any one of claims 3-5, or a pharmaceutically acceptable salt thereof, wherein A² is O.

7. The compound of any one of claims 3-6, or a pharmaceutically acceptable salt thereof, wherein R¹ is unsubstituted alkyl, —COR¹¹, —CON(R¹¹)₂, optionally substituted (C₁-C₄ alkylene)-N(R¹¹)₂, optionally substituted (C₁-C₄ alkylene)N(R¹²)—COR¹¹, optionally substituted (C₁-C₄ alkylene)N(R¹²)—CO₂R¹¹, or optionally substituted (C₁-C₄ alkylene)N(R¹²)—CON(R¹¹)₂.

8. The compound of any one of claims 3-7, or a pharmaceutically acceptable salt thereof, wherein R¹ is —COR¹¹, —CON(R¹¹)₂, optionally substituted (C₁-C₄ alkylene)-N(R¹¹)₂, or optionally substituted (C₁-C₄ alkylene)N(R¹²)—COR¹¹.

9. The compound of any one of claims 3-8, or a pharmaceutically acceptable salt thereof, wherein R¹ is optionally substituted (C₁-C₄ alkylene)-N(R¹¹)₂.

10. The compound of any one of claims 3-9, or a pharmaceutically acceptable salt thereof, wherein R¹ is optionally substituted (C₁-C₄ alkylene)N(R¹²)—COR¹¹.

11. The compound of either of claim 9 or 10, or a pharmaceutically acceptable salt thereof, wherein optionally substituted (C₁-C₄ alkylene) is —CH₂—.

12. The compound of any one of claims 3-11, or a pharmaceutically acceptable salt thereof, wherein each R¹¹ is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl.

13. The compound of claim 12, or a pharmaceutically acceptable salt thereof, wherein R¹¹ is vinyl, propan-2-yl, methyl, ethyl, cyclopropyl, cyclopentyl, azentidin-1-yl or allyl.

14. The compound of claim 12, or a pharmaceutically acceptable salt thereof, wherein R² is H.

15. The compound of claim 12, or a pharmaceutically acceptable salt thereof, wherein R³ is H.

16. The compound of claim 12, or a pharmaceutically acceptable salt thereof, wherein R⁴ is H.

17. The compound of any one of claims 3-16, or a pharmaceutically acceptable salt thereof, wherein n is 0 or 1.

18. The compound of any one of claims 3-17, or a pharmaceutically acceptable salt thereof, wherein m is 0 or 1.

19. The compound of any one of claims 3-18, or a pharmaceutically acceptable salt thereof, wherein X is halogen.

20. The compound of any one of claims 3-19, or a pharmaceutically acceptable salt thereof, wherein Y is halogen.

21. The compound of any one of claims 3-20, or a pharmaceutically acceptable salt thereof, wherein Z is -L-G, optionally substituted (C₁-C₄ alkylene)-OCON(R¹³)₂, optionally substituted (C₁-C₄ alkylene)-N(R¹⁴)CON(R¹³)₂, or optionally substituted (C₁-C₄ alkylene)-SO₂N(R¹³)₂.

22. The compound of claim 21, or a pharmaceutically acceptable salt thereof, wherein Z is -L-G.

23. The compound of any one of claims 3-22, or a pharmaceutically acceptable salt thereof, wherein Z is morpholinomethyl, (8-oxa-3-azabicyclo[3.2.1]octan-3-yl)methyl, (2-oxa-5-azabicyclo[2.2.1]heptan-5-yl)methyl, (1,1-dioxidothiomorpholino)methyl, (oxetan-3-ylamino)methyl, ((methoxycyclobutyl)amino)methyl, (methylpiperazin-1-yl)methyl, (cyanopyrrolidin-1-yl)methyl, or (methoxypyrrolidin-1-yl)methyl.

24. The compound of claim 22, or a pharmaceutically acceptable salt thereof, wherein L is a bond or optionally substituted C₁-C₄ alkylene.

25. The compound of claim 24, or a pharmaceutically acceptable salt thereof, wherein L is an optionally substituted C₁-C₄ alkylene.

26. The compound of claim 25, or a pharmaceutically acceptable salt thereof, wherein L is —CH₂—.

27. The compound of claim 26, or a pharmaceutically acceptable salt thereof, wherein G is optionally substituted heterocyclyl.

28. The compound of claim 27, or a pharmaceutically acceptable salt thereof, wherein G is optionally substituted morpholinyl, piperidinyl, piperazinyl, pyrolidinyl, imidazolyl, or imidazolidinyl.

29. The compound of claim 28, or a pharmaceutically acceptable salt thereof, wherein G is optionally substituted morpholinyl.

30. The compound of claim 29, or a pharmaceutically acceptable salt thereof, wherein G is unsubstituted morpholinyl

31. The compound of any one of claims 3-30, or a pharmaceutically acceptable salt thereof, wherein each R¹³ is independently H, optionally substituted alkyl, optionally substituted alkenyl, optionally substituted carbocyclyl, optionally substituted carbocyclylalkyl, optionally substituted heterocyclyl, optionally substituted heterocyclylalkyl, optionally substituted heteroaryl, or optionally substituted heteroarylalkyl; or two R¹³ groups together with the nitrogen to which they are attached join to form an optionally substituted N-heterocyclyl.

32. A pharmaceutical composition comprising a compound of Formula (I) or (Ia) as described in any one of claims 1-31, or a pharmaceutically acceptable salt thereof, and a pharmaceutically acceptable excipient.

33. A method of treating a gram-negative bacterial infection in a patient in need thereof comprising administering to the patient a pharmaceutical composition comprising a compound of Formula (I) or (Ia) as described in any one of claims 1-32, or a pharmaceutically acceptable salt thereof, and a pharmaceutically acceptable excipient.

34. The method of claim 33, wherein the gram-negative bacterial infection is selected from pneumonia, sepsis, cystic fibrosis, intra-abdominal infection, skin infection and urinary tract infection.

35. The method of claim 33, wherein the gram-negative bacterial infection is selected from chronic urinary tract infection, complicated urinary tract infection, cystitis, pyelonephritis, urethritis, recurrent urinary tract infections, bladder infections, urethral infections and kidney infections.

36. The method of claim 35, wherein the gram-negative bacterial infection is chronic urinary tract infections.

37. The method of claim 35, wherein the gram-negative bacterial infection is complicated urinary tract infections.

38. The method of claim 33, wherein the administration is to treat an existing infection.

39. The method of claim 33, wherein the administration is provided as prophylaxis.
